(19) United States
(12) Patent Application Publication
(10) Pub. No.: US 2025/0163077 A1
(43) Pub. Date: May 22, 2025

(54) MEMBRANE-ASSOCIATED TYROSINE- AND THREONINE-SPECIFIC CDC2-INHIBITORY KINASE (PKMYT1) INHIBITORS AND USES THEREOF

(71) Applicant: Insilico Medicine IP Limited, Hong Kong (CN)

(72) Inventors: Xiao DING, Shanghai (CN); Feng REN, Shanghai (CN); Yazhou WANG, Shanghai (CN); Min ZHENG, Shanghai (CN)

(21) Appl. No.: 18/837,847

(22) PCT Filed: Feb. 17, 2023
(86) PCT No.: PCT/CN2023/076742

§ 371 Date: Aug. 12, 2024

(30) Foreign Application Priority Data
Feb. 18, 2022 (WO) PCT/CN2022/076942

(51) Int. Cl. C07D 519/00; A61K 31/437; A61K 31/4985; A61K 31/519

(52) U.S. Cl. CPC C07D 519/00; A61K 31/437; A61K 31/4985; A61K 31/519

(57) ABSTRACT

Described herein are PKMYT1 (Myt1) inhibitors and pharmaceutical compositions comprising said inhibitors. The subject compounds and compositions are useful for the treatment of a disease or disorder associated with PKMYT1.

### CROSS-REFERENCE

This patent application claims the benefit of International Application No. PCT/CN2022/076942, filed Feb. 18, 2022; which is incorporated herein by reference in its entirety.

### BACKGROUND

PKMYT1 (or Myt1) is a member of the Wee family and was first reported as a kinase capable of phosphorylating Cdc2 efficiently on both threonine-14 and tyrosine-15 in a Xenopus frog. PKMYT1 inhibits cell cycle progression by inhibiting the activities of cell cycle-associated proteins, such as Cyclin A, CDK1, and CDK2. PKMYT1 also drives the progression of a variety of tumors.

The inhibitory phosphorylation of cdc2 is important for the timing of entry into mitosis. Entry into mitosis is initiated by the M phase-promoting factor (MPF), a complex containing the cdc2 protein kinase and cyclin B. Proper regulation of MPF ensures that mitosis occurs only after earlier phases of the cell cycle are complete. Phosphorylation of cdc2 at Tyr-15 and Thr-14 suppresses this activity during interphase (G1, S, and G2). At G2-M transition, cdc2 is dephosphorylated at Tyr-15 and Thr-14 allowing MPF to phosphorylate its mitotic substrates.

Studies have shown that premature activation of cdc2 leads to mitotic catastrophe and cell death. Inhibition of Myt1 is predicted to cause premature activation of cdc2, and thus would kill rapidly proliferating cells. In addition, Myt1 inhibition is predicted to reduce resistance to conventional DNA-damaging chemotherapeutics, because the mechanisms by which cells avoid death involve arrest in the G2 phase of the cell cycle, and repair or DNA damage prior to division. That arrest should be prevented by blocking Myt1 inhibitory phosphorylation of cdc2. Thus forcing the cell to enter mitosis prematurely. Myt1 kinase is an important cell cycle regulator, particularly at the G2/M phase. This is due to cell cycle regulation and subsequent repair of damage to DNA or mitotic apparatus, the targets for most effective chemotherapeutic agents. Myt1 kinase offers a point of intervention downstream from these mechanisms by which tumor cells develop resistance. Inhibition of Myt1 could in and of itself have therapeutic benefit in reducing tumor proliferation, and in addition, could be used in conjunction with conventional chemotherapies to overcome drug resistance.

Based on the foregoing, there is a need to identify a potent PKMYT1 (Myt1) kinase inhibitor for the treatment of cancer.

### SUMMARY

Disclosed herein is a compound of Formula (I), or a pharmaceutically acceptable salt thereof:
wherein:

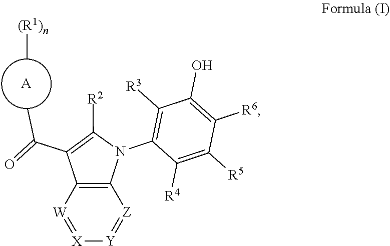

Ring A is heterocycloalkyl or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₅alkyl, C₁-C₅haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1a};
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R; or two R^{1a} on the same atom are taken together to form an oxo;
n is 0-6;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
W is N or CR^{W};
R^{W} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R; or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl; R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (I), the compound is of Formula (Ia):

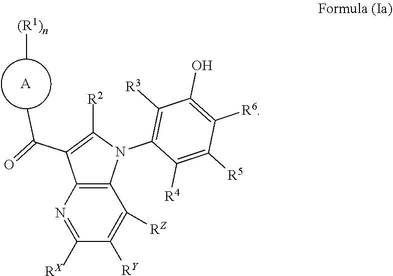

In some embodiments of a compound of Formula (I), the compound is of Formula (Ib):

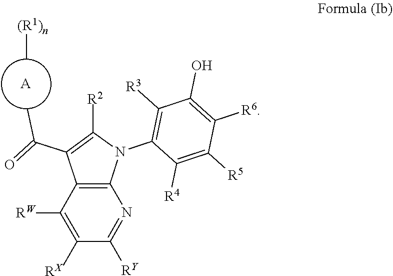

In some embodiments of a compound of Formula (I), the compound is of Formula (Ic):

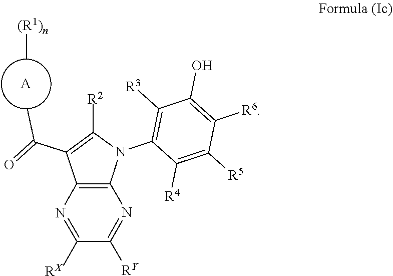

In some embodiments of a compound of Formula (I), the compound is of Formula (Id):

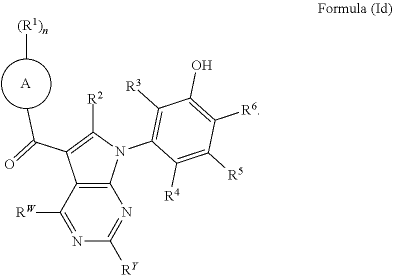

In some embodiments of a compound of Formula (I), the compound is of Formula (Ie):

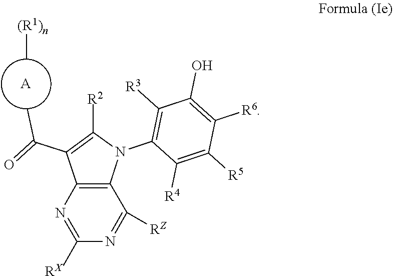

Also disclosed herein is a pharmaceutical composition comprising a compound disclosed herein, or a pharmaceutically acceptable salt thereof, and a pharmaceutically acceptable excipient.

Also disclosed herein is a method of treating cancer in a subject in need thereof, the method comprising administering to the subject a compound disclosed herein, or a pharmaceutically acceptable salt thereof.

Also disclosed herein is a method of modulating PKMYT1 in a subject, the method comprising administering to the subject the compound disclosed herein, or a pharmaceutically acceptable salt thereof.

Also disclosed herein is a method of inhibiting PKMYT1 in a subject, the method comprising administering to the subject the compound disclosed herein, or a pharmaceutically acceptable salt thereof.

Also disclosed herein is a method of selectively inhibiting PKMYT1 (e.g., over WEE1) in a subject, the method comprising administering to the subject the compound disclosed herein, or a pharmaceutically acceptable salt thereof. In some embodiments, compounds described herein selectively inhibit PKMYT1 without inhibiting WEE1. In some embodiments, the compound or a pharmaceutically acceptable salt thereof does not inhibit WEE1. In some embodiments, the compound or a pharmaceutically acceptable salt thereof has a WEE1 IC₅₀ value of at least about 10,000 nM as determined in a WEE1 ADP-Glo assay.

In some embodiments, the subject has cancer. In some embodiments, the cancer depends on the activity of PKMYT1. In some embodiments, the cancer overexpresses CCNE1. In some embodiments, the cancer has an inactivating mutation in the FBXW7 gene. In some embodiments, the cancer is a solid tumor. In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating,” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with PKMYT1” or, alternatively, “a PKMYT1-mediated disease or disorder” means any disease or other deleterious condition in which PKMYT1, or a mutant thereof, is known or suspected to play a role.

As used herein, a “disease or disorder associated with Myt1” or, alternatively, “a Myt1-mediated disease or disorder” means any disease or other deleterious condition in which Myt1, or a mutant thereof, is known or suspected to play a role.

### Compounds

Described herein are compounds, or a pharmaceutically acceptable salt thereof useful in the treatment of a disease or disorder associated with PKMYT1.

Disclosed herein is a compound of Formula (I), or a pharmaceutically acceptable salt thereof:

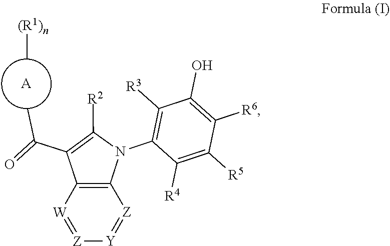

wherein:
Ring A is heterocycloalkyl or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more Ria;
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
W is N or CR^{W};
R^{W} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₅alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₅alkyl, C₁-C₅haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

In some embodiments of a compound of Formula (I), the compound is of Formula (I-1):

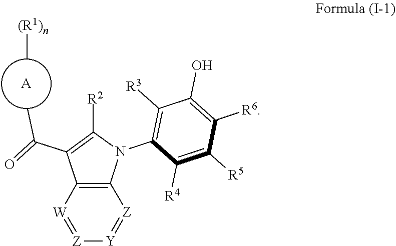

In some embodiments of a compound of Formula (I) or (I-1), X is N. In some embodiments of a compound of Formula (I) or (1-1), X is CR^{X}.

In some embodiments of a compound of Formula (I) or (1-1), Y is N. In some embodiments of a compound of Formula (I) or (1-1), Y is CR^{Y}.

In some embodiments of a compound of Formula (I) or (1-1), W is N. In some embodiments of a compound of Formula (I) or (1-1), W is CR^{W}.

In some embodiments of a compound of Formula (I) or (1-1), Z is N. In some embodiments of a compound of Formula (I) or (1-1), Z is CR^{Z}.

In some embodiments of a compound of Formula (I), the compound is of Formula (Ia):

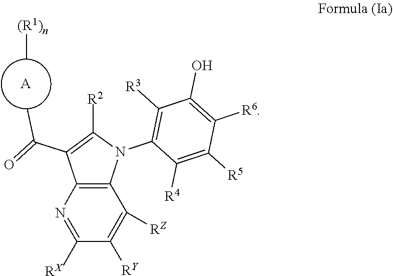

In some embodiments of a compound of Formula (I-1), the compound is of Formula (Ia-1):

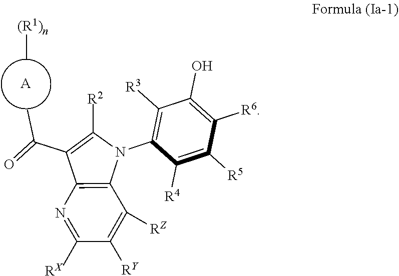

In some embodiments of a compound of Formula (I), the compound is of Formula (Ib):

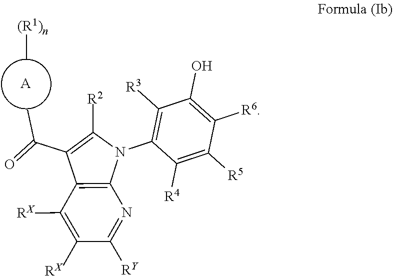

In some embodiments of a compound of Formula (I-1), the compound is of Formula (Ib-1):

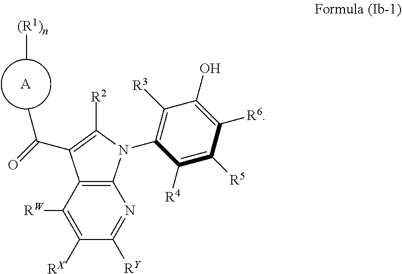

In some embodiments of a compound of Formula (I), the compound is of Formula (Ic):

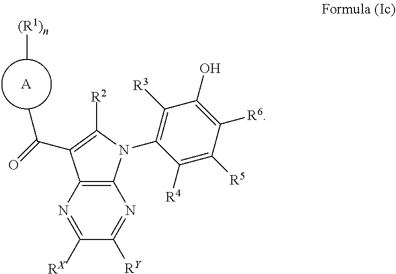

In some embodiments of a compound of Formula (I-1), the compound is of Formula (Ic-1):

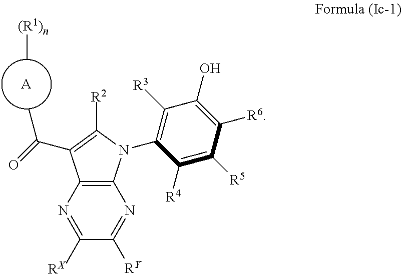

In some embodiments of a compound of Formula (I), the compound is of Formula (Id):

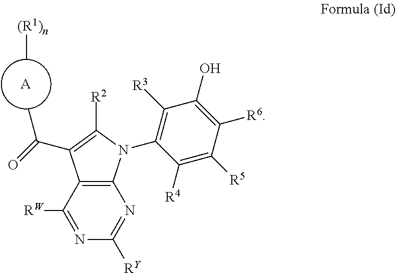

In some embodiments of a compound of Formula (I-1), the compound is of Formula (Id-1):

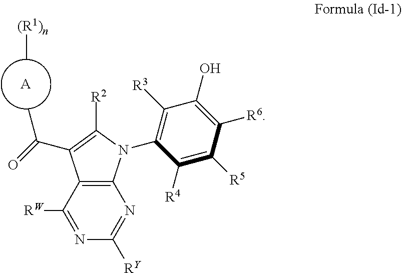

In some embodiments of a compound of Formula (I), the compound is of Formula (Ie):

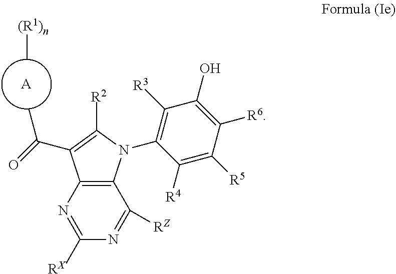

In some embodiments of a compound of Formula (I-1), the compound is of Formula (Ie-1):

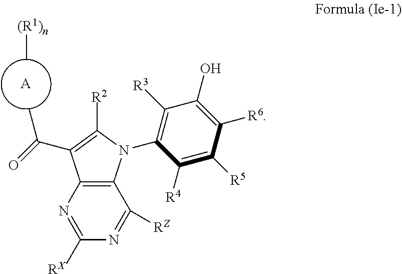

In some embodiments of a compound of Formula (I), (I-1), (Ia)-(Ie), or (Ia-1)-(Ie-1), R³ is halogen, C₁-C₆alkyl, or C₁-C₆haloalkyl. In some embodiments of a compound of Formula (I), (I-1), (Ia)-(Ie), or (Ia-1)-(Ie-1), R³ is halogen or C₁-C₆alkyl. In some embodiments of a compound of Formula (I), (I-1), (Ia)-(Ie), or (Ia-1)-(Ie-1), R³ is C₁-C₆alkyl. In some embodiments of a compound of Formula (I), (I-1), (Ia)-(Ie), or (Ia-1)-(Ie-1), R³ is C₁-C₃alkyl. In some embodiments of a compound of Formula (I), (I-1), (Ia)-(Ie), or (Ia-1)-(Ie-1), R³ is n-propyl or isopropyl. In some embodiments of a compound of Formula (I), (I-1), (Ia)-(Ie), or (Ia-1)-(Ie-1), R³ is ethyl. In some embodiments of a compound of Formula (I), (I-1), (Ia)-(Ie), or (Ia-1)-(Ie-1), R³ is methyl.

In some embodiments of a compound of Formula (I), (I-1), (Ia)-(Ie), or (Ia-1)-(Ie-1), R⁴ is halogen, C₁-C₆alkyl, or C₁-C₆haloalkyl. In some embodiments of a compound of Formula (I), (I-1), (Ia)-(Ie), or (Ia-1)-(Ie-1), R⁴ is halogen or C₁-C₆alkyl. In some embodiments of a compound of Formula (I), (I-1), (Ia)-(Ie), or (Ia-1)-(Ie-1), R⁴ is C₁-C₆alkyl. In some embodiments of a compound of Formula (I), (1-1), (Ia)-(Ie), or (Ia-1)-(Ie-1), R⁴ is C₁-C₃alkyl. In some embodiments of a compound of Formula (I), (I-1), (Ia)-(Ie), or (Ia-1)-(Ie-1), R⁴ is n-propyl or isopropyl. In some embodiments of a compound of Formula (I), (I-1), (Ia)-(Ie), or (Ia-1)-(Ie-1), R⁴ is ethyl. In some embodiments of a compound of Formula (I), (I-1), (Ia)-(Ie), or (Ia-1)-(Ie-1), R⁴ is methyl.

In some embodiments of a compound of Formula (I), (I-1), (Ia)-(Ie), or (Ia-1)-(Ie-1), R⁵ is hydrogen, halogen, C₁-C₆alkyl, or C₁-C₆haloalkyl. In some embodiments of a compound of Formula (I), (I-1), (Ia)-(Ie), or (Ia-1)-(Ie-1), R⁵ is hydrogen, halogen, C₁-C₃alkyl, or C₁-C₃haloalkyl. In some embodiments of a compound of Formula (I), (I-1), (Ia)-(Ie), or (Ia-1)-(Ie-1), R⁵ is hydrogen.

In some embodiments of a compound of Formula (I), (I-1), (Ia)-(Ie), or (Ia-1)-(Ie-1), R⁶ is hydrogen, halogen, C₁-C₆alkyl, or C₁-C₆haloalkyl. In some embodiments of a compound of Formula (I), (I-1), (Ia)-(Ie), or (Ia-1)-(Ie-1), R⁶ is hydrogen, halogen, C₁-C₃alkyl, or C₁-C₃haloalkyl. In some embodiments of a compound of Formula (I), (I-1), (Ia)-(Ie), or (Ia-1)-(Ie-1), R⁶ is hydrogen.

In some embodiments of a compound of Formula (I), (I-1), (Ia)-(Ie), or (Ia-1)-(Ie-1), R² is halogen, —CN, —OH, —OR^{a}, —NR^{c}R^{d}, C₁-C₆alkyl, or C₁-C₆haloalkyl. In some embodiments of a compound of Formula (I), (I-1), (Ia)-(Ie), or (Ia-1)-(Ie-1), R² is —OH, —OR^{a}, or —NR^{c}R^{d}. In some embodiments of a compound of Formula (I), (I-1), (Ia)-(Ie), or (Ia-1)-(Ie-1), R² is —NR^{c}R^{d}. In some embodiments of a compound of Formula (I), (I-1), (Ia)-(Ie), or (Ia-1)-(Ie-1), R² is —NH(C₁-C₆alkyl). In some embodiments of a compound of Formula (I), (I-1), (Ia)-(Ie), or (Ia-1)-(Ie-1), R² is NH₂.

In some embodiments of a compound of Formula (I), (I-1), (Ib), (Id), (Ib-1), or (Id-1), R^{W} is hydrogen, halogen, —CN, —OH, —ORₐ, —NR^{c}R^{d}, C₁-C₆alkyl, C₁-C₆haloalkyl, C₁-C₆hydroxyalkyl, C₁-Csaminoalkyl, C₁-C₆heteroalkyl, cycloalkyl, or heterocycloalkyl; wherein the alkyl, cycloalkyl, and heterocycloalkyl is optionally substituted with one or more R. In some embodiments of a compound of Formula (I), (I-1), (Ib), (Id), (Ib-1), or (Id-1), R^{W} is hydrogen, halogen, —CN, —OH, —ORₐ, —NR^{c}R^{d}, C₁-C₆alkyl, or C₁-C₆haloalkyl; wherein the alkyl is optionally substituted with one or more R. In some embodiments of a compound of Formula (I), (I-1), (Ib), (Id), (Ib-1), or (Id-1), R^{W} is hydrogen, halogen, —CN, —OH, —OR, —NR^{c}R^{d}, C₁-C₆alkyl, or C₁-C₆haloalkyl. In some embodiments of a compound of Formula (I), (I-1), (Ib), (Id), (Ib-1), or (Id-1), R^{W} is hydrogen, halogen, C₁-C₆alkyl, or C₁-C₆haloalkyl. In some embodiments of a compound of Formula (I), (I-1), (Ib), (Id), (Ib-1), or (Id-1), R^{W} is hydrogen, halogen, or C₁-C₆alkyl. In some embodiments of a compound of Formula (I), (I-1), (Ib), (Id), (Ib-1), or (Id-1), R^{W} is hydrogen, halogen, or C₁-C₃alkyl. In some embodiments of a compound of Formula (I), (I-1), (Ib), (Id), (Ib-1), or (Id-1), R^{W} is hydrogen or C₁-C₃alkyl. In some embodiments of a compound of Formula (I), (I-1), (Ib), (Id), (Ib-1), or (Id-1), R^{W} is hydrogen.

In some embodiments of a compound of Formula (I), (I-1), (Ia), (Ic), (Ie), (Ia-1), (Ic-1), or (Ie-1), R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, alkenyl, alkynyl, cycloalkyl, and heterocycloalkyl is optionally substituted with one or more R^{Xa}

In some embodiments of a compound of Formula (I), (I-1), (Ia), (Ic), (Ie), (Ia-1), (Ic-1), or (Ie-1), R^{X} is hydrogen, halogen, C₁-C₆alkyl, C₁-C₆haloalkyl, C₁-C₆hydroxyalkyl, C₂-C₆alkenyl, C₂-C₅alkynyl, cycloalkyl, or heterocycloalkyl; wherein the alkyl, alkenyl, cycloalkyl, and heterocycloalkyl is optionally substituted with one or more R^{Xa}

In some embodiments of a compound of Formula (I), (I-1), (Ia), (Ic), (Ie), (Ia-1), (Ic-1), or (Ie-1), R^{X} is hydrogen, halogen, C₁-C₆alkyl, C₁-C₆haloalkyl, C₁-C₆hydroxyalkyl, C₂-C₆alkenyl, C₂-C₅alkynyl, cycloalkyl, or heterocycloalkyl.

In some embodiments of a compound of Formula (I), (I-1), (Ia), (Ic), (Ie), (Ia-1), (Ic-1), or (Ie-1), R^{X} is hydrogen, halogen, C₁-C₆alkyl, C₁-C₆haloalkyl, C₂-C₆alkenyl, C₂-C₆alkynyl, cycloalkyl, or heterocycloalkyl.

In some embodiments of a compound of Formula (I), (I-1), (Ia), (Ic), (Ie), (Ia-1), (Ic-1), or (Ie-1), R^{X} is cycloalkyl. In some embodiments of a compound of Formula (I), (I-1), (Ia), (Ic), (Ie), (Ia-1), (Ic-1), or (Ie-1), R^{X} is cyclopropyl.

In some embodiments of a compound of Formula (I), (I-1), (Ia), (Ic), (Ie), (Ia-1), (Ic-1), or (Ie-1), R^{X} is heterocycloalkyl.

In some embodiments of a compound of Formula (I), (I-1), (Ia), (Ic), (Ie), (Ia-1), (Ic-1), or (Ie-1), R^{X} is hydrogen, halogen, —CN, —OH, —OR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally substituted with one or more R^{Xa}. In some embodiments of a compound of Formula (I), (I-1), (Ia), (Ic), (Ie), (Ia-1), (Ic-1), or (Ie-1), R^{X} is hydrogen, halogen, C₁-C₆alkyl, C₁-C₆haloalkyl, or heterocycloalkyl; wherein the alkyl and heterocycloalkyl is optionally substituted with one or more R^{Xa}. In some embodiments of a compound of Formula (I), (I-1), (Ia), (Ic), (Ie), (Ia-1), (Ic-1), or (Ie-1), R^{X} is optionally substituted cycloalkyl or heterocycloalkyl. In some embodiments of a compound of Formula (I), (I-1), (Ia), (Ic), (Ie), (Ia-1), (Ic-1), or (Ie-1), R^{X} is optionally substituted 5- or 6-membered heterocycloalkyl. In some embodiments of a compound of Formula (I), (I-1), (Ia), (Ic), (Ie), (Ia-1), (Ic-1), or (Ie-1), R^{X} is hydrogen, halogen, C₁-C₆alkyl, or C₁-C₆haloalkyl. In some embodiments of a compound of Formula (I), (I-1), (Ia), (Ic), (Ie), (Ia-1), (Ic-1), or (Ie-1), R^{X} is halogen, C₁-C₆alkyl, or C₁-C₆haloalkyl. In some embodiments of a compound of Formula (I), (I-1), (Ia), (Ic), (Ie), (Ia-1), (Ic-1), or (Ie-1), R^{X} is C₁-C₆haloalkyl. In some embodiments of a compound of Formula (I), (I-1), (Ia), (Ic), (Ie), (Ia-1), (Ic-1), or (Ie-1), R^{X} is C₁-C₃haloalkyl. In some embodiments of a compound of Formula (I), (I-1), (Ia), (Ic), (Ie), (Ia-1), (Ic-1), or (Ie-1), R^{X} is CF₃. In some embodiments of a compound of Formula (I), (I-1), (Ia), (Ic), (Ie), (Ia-1), (Ic-1), or (Ie-1), R^{X} is halogen or C₁-C₆alkyl. In some embodiments of a compound of Formula (I), (I-1), (Ia), (Ic), (Ie), (Ia-1), (Ic-1), or (Ie-1), R^{X} is halogen or C₁-C₃alkyl. In some embodiments of a compound of Formula (I), (I-1), (Ia), (Ic), (Ie), (Ia-1), (Ic-1), or (Ie-1), R^{X} is halogen. In some embodiments of a compound of Formula (I), (I-1), (Ia), (Ic), (Ie), (Ia-1), (Ic-1), or (Ie-1), R^{X} is C₁-C₃alkyl. In some embodiments of a compound of Formula (I), (I-1), (Ia), (Ic), (Ie), (Ia-1), (Ic-1), or (Ie-1), R^{X} is methyl.

In some embodiments of a compound of Formula (I), (I-1), (Ia), (Ic), (Ie), (Ia-1), (Ic-1), or (Ie-1), each R^{Xa} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R; or two R^{Xa} on the same atom are taken together to form an oxo. In some embodiments of a compound of Formula (I), (I-1), (Ia), (Ic), (Ie), (Ia-1), (Ic-1), or (Ie-1), each R^{Xa} is independently halogen, —CN, —OH, —OR^{a}, —NR^{c}R^{d}, C₁-C₆alkyl, C₁-C₆haloalkyl, or C₁-C₆heteroalkyl; or two R^{Xa} on the same atom are taken together to form an oxo. In some embodiments of a compound of Formula (I), (I-1), (Ia), (Ic), (Ie), (Ia-1), (Ic-1), or (Ie-1), each R^{Xa} is independently halogen, C₁-C₆alkyl, C₁-C₆haloalkyl, or C₁-C₆heteroalkyl; or two R^{Xa} on the same atom are taken together to form an oxo. In some embodiments of a compound of Formula (I), (I-1), (Ia), (Ic), (Ie), (Ia-1), (Ic-1), or (Ie-1), one or more R^{Xa} is halogen, —OR^{a}, optionally substituted C₁-C₆alkyl, optionally substituted C₁-C₆haloalkyl, or optionally substituted C₁-C₆heteroalkyl.

In some embodiments of a compound of Formula (I), (I-1), (Ia), (Ic), (Ie), (Ia-1), (Ic-1), or (Ie-1), R^{X} is

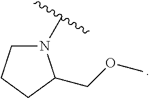 [SMILES: *N1CCCC1COC]

In some embodiments of a compound of Formula (I), (I-1), (Ia)-(Id), or (Ia-1)-(Id-1), R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R^{Ya}.

In some embodiments of a compound of Formula (I), (I-1), (Ia)-(Id), or (Ia-1)-(Id-1), R^{Y} is hydrogen, halogen, C₁-C₆alkyl, C₁-C₆haloalkyl, cycloalkyl, or heterocycloalkyl; wherein the alkyl, cycloalkyl, and heterocycloalkyl is optionally substituted with one or more R^{Ya}.

In some embodiments of a compound of Formula (I), (I-1), (Ia)-(Id), or (Ia-1)-(Id-1), R^{Y} is hydrogen, halogen, —CN, —OH, —OR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally substituted with one or more R^{Ya}. In some embodiments of a compound of Formula (I), (I-1), (Ia)-(Id), or (Ia-1)-(Id-1), R^{Y} is hydrogen, halogen, C₁-C₆alkyl, or heterocycloalkyl; wherein the alkyl and heterocycloalkyl is optionally substituted with one or more R^{Ya}. In some embodiments of a compound of Formula (I), (I-1), (Ia)-(Id), or (Ia-1)-(Id-1), R^{Y} is hydrogen, C₁-C₆alkyl or heterocycloalkyl; wherein the alkyl and heterocycloalkyl is optionally substituted with one or more R^{Ya}. In some embodiments of a compound of Formula (I), (I-1), (Ia)-(Id), or (Ia-1)-(Id-1), R^{Y} is heterocycloalkyl optionally substituted with one or more R^{Ya}. In some embodiments of a compound of Formula (I), (I-1), (Ia)-(Id), or (Ia-1)-(Id-1), R^{Y} is 5-membered ring. In some embodiments of a compound of Formula (I), (I-1), (Ia)-(Id), or (Ia-1)-(Id-1), R^{Y} is 6-membered ring. In some embodiments of a compound of Formula (I), (I-1), (Ia)-(Id), or (Ia-1)-(Id-1), R is hydrogen or C₁-C₆alkyl. In some embodiments of a compound of Formula (I), (I-1), (Ia)-(Id), or (Ia-1)-(Id-1), R^{Y} is hydrogen or C₁-C₃alkyl. In some embodiments of a compound of Formula (I), (I-1), (Ia)-(Id), or (Ia-1)-(Id-1), R^{Y} is C₁-C₆alkyl. In some embodiments of a compound of Formula (I), (I-1), (Ia)-(Id), or (Ia-1)-(Id-1), R^{Y} is C₁-C₃alkyl. In some embodiments of a compound of Formula (I), (I-1), (Ia)-(Id), or (Ia-1)-(Id-1), R^{Y} is methyl. In some embodiments of a compound of Formula (I), (I-1), (Ia)-(Id), or (Ia-1)-(Id-1), R^{Y} is C₁-C₆haloalkyl. In some embodiments of a compound of Formula (I), (I-1), (Ia)-(Id), or (Ia-1)-(Id-1), R^{Y} is C₁-C₃haloalkyl. In some embodiments of a compound of Formula (I), (I-1), (Ia)-(Id), or (Ia-1)-(Id-1), R^{Y} is CF₃.

In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R; or two R^{Ya} on the same atom are taken together to form an oxo.

In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl.

In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, C₁-C₆alkyl, C₁-C₆haloalkyl, or C₁-C₆heteroalkyl.

In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R; or two R^{Ya} on the same atom are taken together to form an oxo. In some embodiments of a compound of Formula (I), (I-1), (Ia)-(Id), or (Ia-1)-(Id-1), each R^{Ya} is independently halogen, —CN, —OH, —OR^{a}, —NR^{c}R^{d}, C₁-C₆alkyl, C₁-C₆haloalkyl, or C₁-C₆heteroalkyl; or two R^{Ya} on the same atom are taken together to form an oxo. In some embodiments of a compound of Formula (I), (I-1), (Ia)-(Id), or (Ia-1)-(Id-1), each R^{Ya} is independently halogen, —OH, —OR^{a}, —NR^{c}R^{d}, C₁-C₆alkyl, or C₁-C₆haloalkyl; or two R^{Ya} on the same atom are taken together to form an oxo. In some embodiments of a compound of Formula (I), (I-1), (Ia)-(Id), or (Ia-1)-(Id-1), one or more R^{Ya} is independently halogen, —OH, —OR^{a}, —NR^{c}R^{d}, optionally substituted C₁-C₆alkyl, optionally substituted C₁-C₆haloalkyl, or optionally substituted C₁-C₆heteroalkyl; or two R^{Ya} on the same atom are taken together to form an oxo. In some embodiments of a compound of Formula (I), (I-1), (Ia)-(Id), or (Ia-1)-(Id-1), one or more R^{Ya} is —OR^{a}. In some embodiments of a compound of Formula (I), (I-1), (Ia)-(Id), or (Ia-1)-(Id-1), one or more R^{Ya} is optionally substituted C₁-C₆heteroalkyl.

In some embodiments of a compound of Formula (I), (I-1), (Ia)-(Id), or (Ia-1)-(Id-1), R^{Y} is

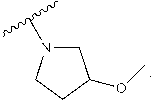 [SMILES: *N1CCC(OC)C1]

In some embodiments of a compound of Formula (I), (I-1), (Ia)-(Id), or (Ia-1)-(Id-1), R^{Y} is

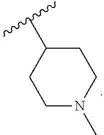 [SMILES: *C1CCN(C)CC1]

In some embodiments of a compound of Formula (I), (I-1), (1a)-(Id), or (Ia-1)-(Id-1), R^{Y} is

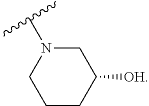 [SMILES: *N1CCC[C@@H](O)C1]

In some embodiments of a compound of Formula (I), (I-1), (Ia)-(Id), or (Ia-1)-(Id-1), R^{Y} is

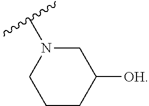 [SMILES: *N1CCCC(O)C1]

In some embodiments of a compound of Formula (I), (I-1), (Ia), (Ib), (Ie), (Ia-1), (Ib-1), or (Ie-1), R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R. In some embodiments of a compound of Formula (I), (I-1), (Ia), (Ib), (Ie), (Ia-1), (Ib-1), or (Ie-1), R^{Z} is hydrogen, halogen, —CN, —OH, —OR^{a}, —NR^{C}R^{d}, C₁-C₆alkyl, or C₁-C₆haloalkyl; wherein the alkyl is optionally substituted with one or more R. In some embodiments of a compound of Formula (I), (I-1), (Ia), (Tb), (Ie), (Ia-1), (Ib-1), or (Ie-1), R^{Z} is hydrogen, halogen, —CN, —OH, —OR^{a}, —NR^{c}R^{d}, C₁-C₆alkyl, or C₁-C₆haloalkyl. In some embodiments of a compound of Formula (I), (I-1), (Ia), (Tb), (Ie), (Ia-1), (Ib-1), or (Ie-1), R^{Z} is hydrogen, halogen, C₁-C₆alkyl, or C₁-C₆haloalkyl. In some embodiments of a compound of Formula (I), (I-1), (Ia), (Ib), (Ie), (Ia-1), (Ib-1), or (Ie-1), R^{Z} is hydrogen, halogen, or C₁-C₆alkyl. In some embodiments of a compound of Formula (I), (I-1), (Ia), (Ib), (Ie), (Ia-1), (Ib-1), or (Ie-1), R^{Z} is hydrogen, halogen, or C₁-C₃alkyl. In some embodiments of a compound of Formula (I), (I-1), (Ia), (Ib), (Ie), (Ia-1), (Ib-1), or (Ie-1), R^{Z} is hydrogen or C₁-C₃alkyl. In some embodiments of a compound of Formula (I), (I-1), (Ta), (Tb), (Ie), (Ia-1), (Ib-1), or (Ie-1), R^{Z} is hydrogen.

In some embodiments of a compound of Formula (I),

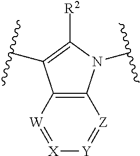

is

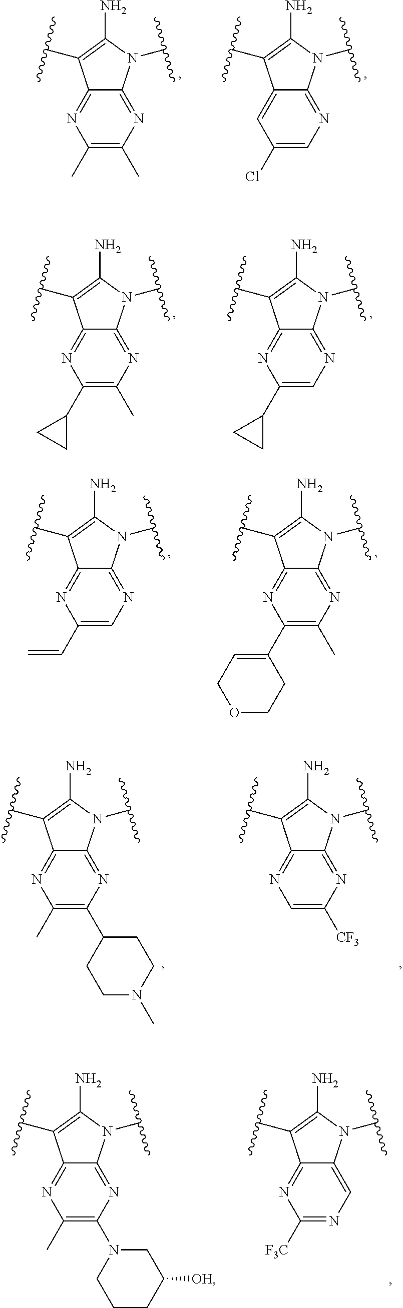

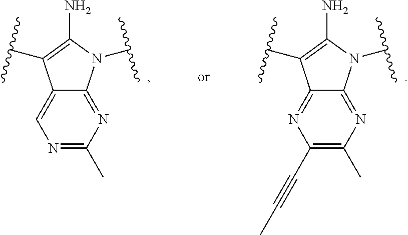 [SMILES: *c1c(N)n(*)c2nc(C)ncc12 ; *c1c(N)n(*)c2nc(C)c(C#CC)nc12]

In some embodiments of a compound of Formula (I),

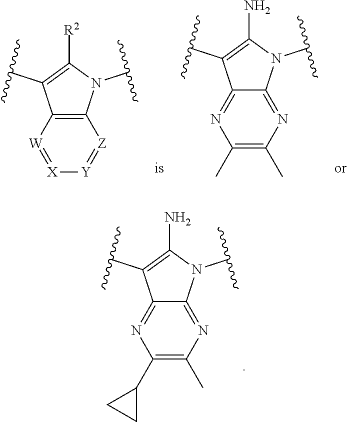

In some embodiments of a compound of Formula (I),

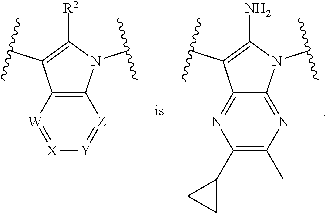

In some embodiments of a compound of Formula (I),

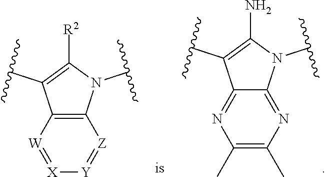

In some embodiments of a compound of Formula (I), (I-1), (Ia), (Ib), (Ie), (Ta-1), (Tb-1), or (Ie-1), Ring A is heteroaryl. In some embodiments of a compound of Formula (I), (I-1), (Ia), (Ib), (Ie), (Ia-1), (Ib-1), or (Ie-1), Ring A is 5- or 6-membered heteroaryl.

In some embodiments of a compound of Formula (I), (I-1), (Ia)-(Ie), or (Ia-1)-(Ie-1), Ring A is heterocycloalkyl. In some embodiments of a compound of Formula (I), (I-1), (Ia)-(Ie), or (Ia-1)-(Ie-1), Ring A is monocyclic heterocycloalkyl. In some embodiments of a compound of Formula (I), (I-1), (Ta)—(Ie), or (Ia-1)-(Ie-1), Ring A is bicyclic heterocycloalkyl. In some embodiments of a compound of Formula (I), (I-1), (Ia)-(Ie), or (Ia-1)-(Ie-1), Ring A is bicyclic heterocycloalkyl comprising a heterocycloalkyl fused to a heteroaryl ring. In some embodiments of a compound of Formula (I), (I-1), (Ia)-(Ie), or (Ia-1)-(Ie-1), Ring A is 6-6 or 6-5 fused bicyclic heterocycloalkyl.

In some embodiments of a compound of Formula (I), (I-1), (Ia)-(Ie), or (Ia-1)-(Ie-1), Ring A is

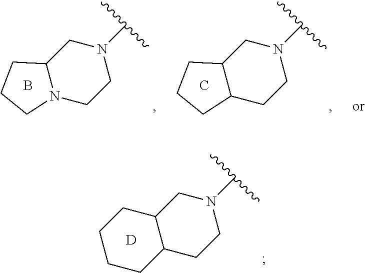

wherein Ring B is a 5-membered heteroaryl, Ring C is a 5-membered heteroaryl, and Ring D is a 6-membered heteroaryl.

In some embodiments of a compound of Formula (I), (I-1), (Ia)-(Ie), or (Ia-1)-(Ie-1), Ring A is

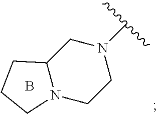

wherein Ring B is a 5-membered heteroaryl. In some embodiments of a compound of Formula (T), (I-1), (Ia)-(Ie), or (Ia-1)-(Ie-1), Ring A is

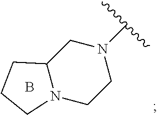

wherein Ring B is pyrrolyl, pyrazolyl, imidazolyl, triazolyl, or tetrazolyl. In some embodiments of a compound of Formula (I), (I-1), (Ia)-(Ie), or (Ia-1)-(Ie-1), Ring A is

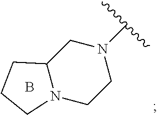

wherein Ring B is pyrazolyl, or triazolyl.

In some embodiments of a compound of Formula (I), (I-1), (Ia)-(Ie), or (Ia-1)-(Ie-1), Ring A is

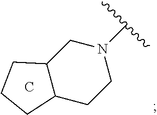

wherein Ring C is a 5-membered heteroaryl. In some embodiments of a compound of Formula (I), (I-1), (Ia)-(Ie), or (Ia-1)-(Ie-1), Ring A is

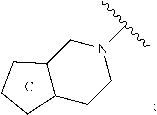

wherein Ring C is pyrrolyl, pyrazolyl, imidazolyl, triazolyl, thiophenyl, furanyl, thiazolyl, isothiazolyl, oxazolyl, or isoxazolyl. In some embodiments of a compound of Formula (I), (I-1), (Ia)-(Ie), or (Ia-1)-(Ie-1), Ring A is

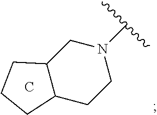

wherein Ring C is pyrazolyl or thiazolyl.

In some embodiments of a compound of Formula (I), (I-1), (Ia)-(Ie), or (Ia-1)-(Ie-1), Ring A is

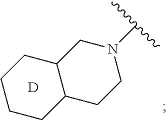

wherein Ring D is a 6-membered heteroaryl. In some embodiments of a compound of Formula (I), (I-1), (Ia)-(Ie), or (Ia-1)-(Ie-1), Ring A is

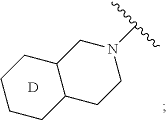

wherein Ring D is pyridinyl, pyrimidinyl, or pyrazinyl. In some embodiments of a compound of Formula (I), (I-1), (Ia)-(Ie), or (Ia-1)-(Ie-1), Ring A is

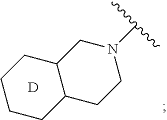

wherein Ring D is pyridinyl.

In some embodiments of a compound of Formula (I), (I-1), (Ia)-(Ie), or (Ia-1)-(Ie-1), Ring A is

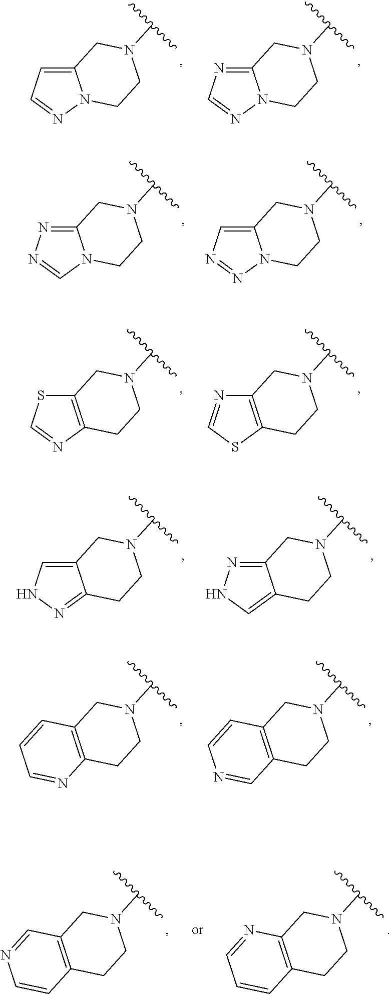

In some embodiments of a compound of Formula (I), (I-1), (Ia)-(Ie), or (Ia-1)-(Ie-1), Ring A is

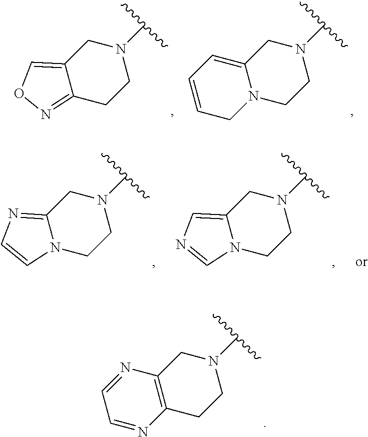 [SMILES: *N1CCc2nocc2C1 ; *N1CCN2CC=CC=C2C1 ; *N1CCn2ccnc2C1 ; *N1CCn2cncc2C1 ; *N1CCc2nccnc2C1]

In some embodiments of a compound of Formula (I), (I-1), (Ia)-(Ie), or (Ia-1)-(Ie-1), Ring A is

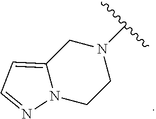 [SMILES: *N1CCn2nccc2C1]

In some embodiments of a compound of Formula (I), (I-1), (Ia)-(Ie), or (Ia-1)-(Ie-1), each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R^{1a}; or two R¹ on the same atom are taken together to form an oxo.

In some embodiments of a compound of Formula (I), (I-1), (Ia)-(Ie), or (Ia-1)-(Ie-1), each R¹ is independently halogen, —CN, —OH, —OR^{a}, —NR^{c}R^{d}, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is independently optionally substituted with one or more R^{1a}; or two R¹ on the same atom are taken together to form an oxo.

In some embodiments of a compound of Formula (I), (I-1), (Ia)-(Ie), or (Ia-1)-(Ie-1), each R¹ is independently halogen, —CN, —OH, —OR^{a}, —NR^{c}R^{d}, C₁-C₆alkyl, C₁-C₆haloalkyl, or C₁-C₆hydroxyalkyl; or two R¹ on the same atom are taken together to form an oxo.

In some embodiments of a compound of Formula (I), (I-1), (Ia)-(Ie), or (Ia-1)-(Ie-1), each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl; wherein each alkyl, cycloalkyl, and heterocycloalkyl is independently optionally substituted with one or more R^{1a}; or two R¹ on the same atom are taken together to form an oxo. In some embodiments of a compound of Formula (I), (I-1), (1a)-(Ie), or (Ia-1)-(Ie-1), each R¹ is independently halogen, —CN, —OH, —OR^{a}, —NR^{c}R^{d}, C₁-C₆alkyl, or C₁-C₆haloalkyl; or two R¹ on the same atom are taken together to form an oxo. In some embodiments of a compound of Formula (I), (I-1), (Ia)-(Ie), or (Ia-1)-(Ie-1), each R¹ is independently halogen, —CN, —OH, —OR^{a}, —NR^{c}R^{d}, C₁-C₆alkyl, or C₁-C₆haloalkyl. In some embodiments of a compound of Formula (I), (I-1), (1a)-(Ie), or (Ia-1)-(Ie-1), each R¹ is independently halogen, C₁-C₆alkyl, or C₁-C₆haloalkyl. In some embodiments of a compound of Formula (I), (I-1), (Ia)-(Ie), or (Ia-1)-(Ie-1), one or more R¹ is independently halogen, C₁-C₆alkyl, or C₁-C₆haloalkyl. In some embodiments of a compound of Formula (I), (I-1), (Ia)-(Ie), or (Ia-1)-(Ie-1), one or more R¹ is independently halogen, C₁-C₃alkyl, or C₁-C₃haloalkyl. In some embodiments of a compound of Formula (I), (I-1), (Ia)-(Ie), or (Ia-1)-(Ie-1), one or more R¹ is independently halogen, methyl, or CF₃.

In some embodiments of a compound of Formula (I), (I-1), (Ia)-(Ie), or (Ia-1)-(Ie-1), each R^{1a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆heteroalkyl, cycloalkyl, or heterocycloalkyl; wherein each alkyl, cycloalkyl, and heterocycloalkyl is independently optionally substituted with one or more R; or two R^{1a} on the same atom are taken together to form an oxo. In some embodiments of a compound of Formula (I), (I-1), (Ia)-(Ie), or (Ia-1)-(Ie-1), each R^{1a} is independently halogen, —CN, —OH, —OR, —NR^{c}R^{d}, C₁-C₆alkyl, or C₁-C₆haloalkyl; or two R^{1a} on the same atom are taken together to form an oxo. In some embodiments of a compound of Formula (I), (I-1), (Ia)-(Ie), or (Ia-1)-(Ie-1), each R^{1a} is independently halogen, C₁-C₆alkyl, or C₁-C₆haloalkyl; or two R^{1a} on the same atom are taken together to form an oxo.

In some embodiments of a compound of Formula (I), (I-1), (Ia)-(Ie), or (Ia-1)-(Ie-1), n is 0-2. In some embodiments of a compound of Formula (I), (I-1), (Ia)-(Ie), or (Ia-1)-(Ie-1), n is 1 or 2. In some embodiments of a compound of Formula (I), (I-1), (Ia)-(Ie), or (Ia-1)-(Ie-1), n is 0. In some embodiments of a compound of Formula (I), (I-1), (Ia)-(Ie), or (Ia-1)-(Ie-1), n is 1. In some embodiments of a compound of Formula (I), (I-1), (Ia)-(Ie), or (Ia-1)-(Ie-1), n is 2.

In some embodiments of a compound of Formula (I), (I-1), (Ia)-(Ie), or (Ia-1)-(Ie-1),

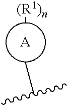

is

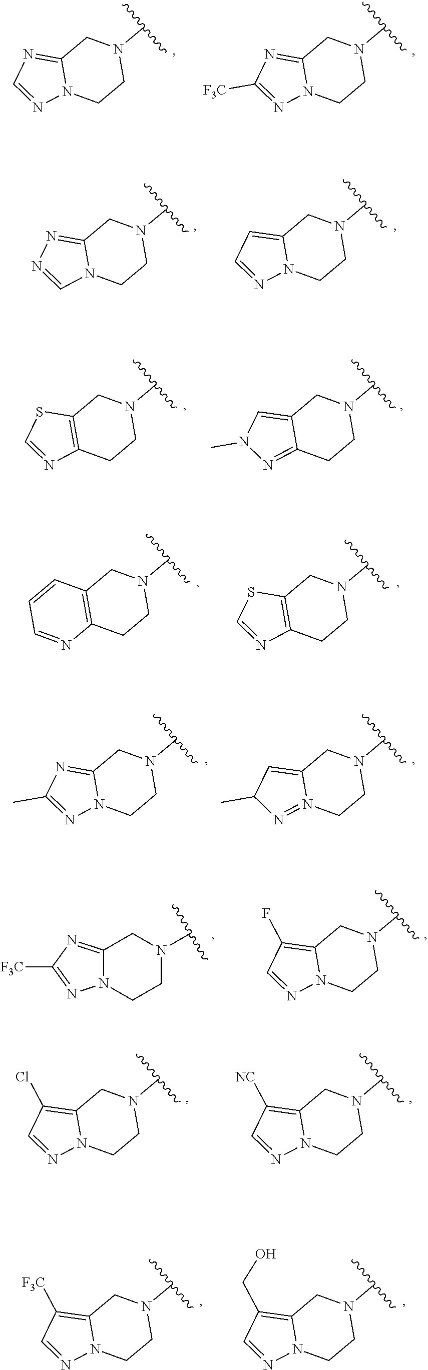

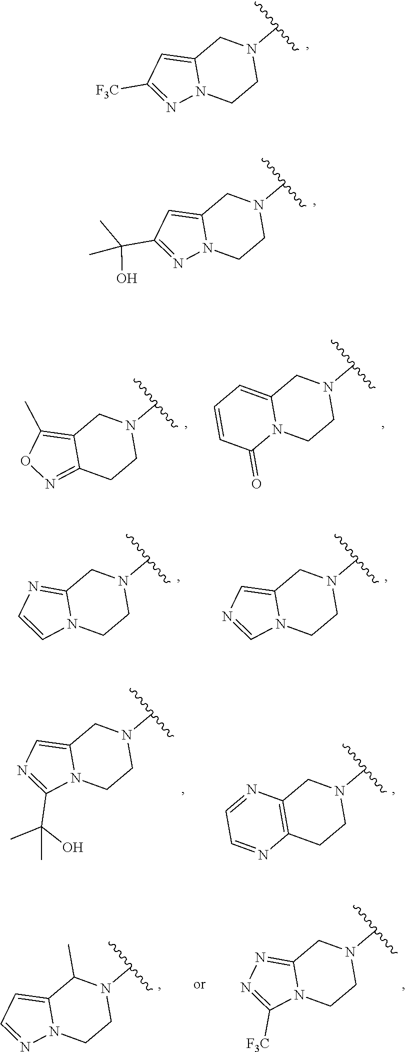

In some embodiments of a compound of Formula (I), (I-1), (Ia)-(Ie), or (Ia-1)-(Ie-1),

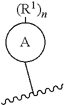

is

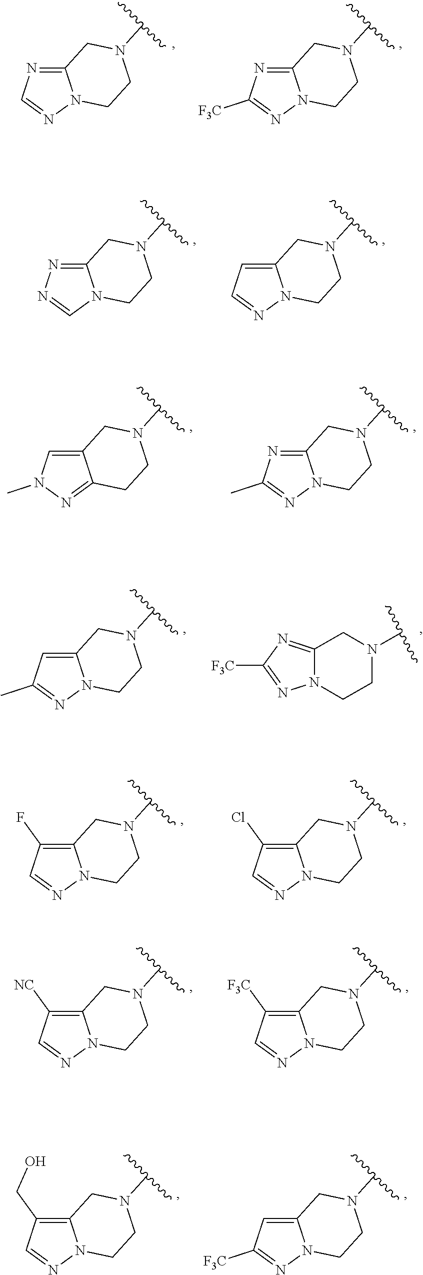

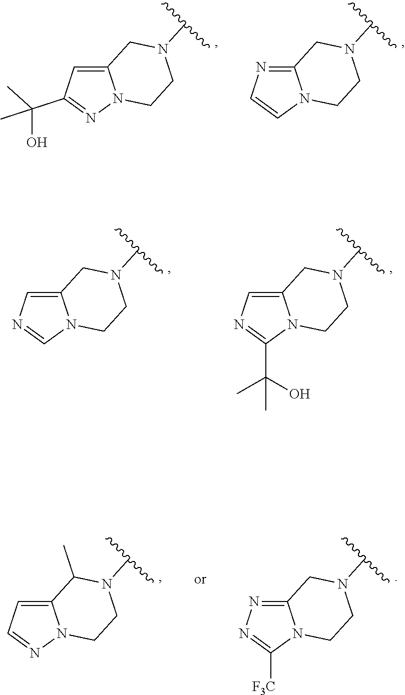 [SMILES: *N1CCn2nc(C(C)(C)O)cc2C1 ; *N1CCn2ccnc2C1 ; *N1CCn2cncc2C1 ; *N1CCn2c(cnc2C(C)(C)O)C1 ; *N1CCn2nccc2C1C ; *N1CCn2c(nnc2C(F)(F)F)C1]

In some embodiments of a compound of Formula (I), (I-1), (Ia)-(Ie), or (Ia-1)-(Ie-1),

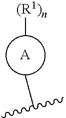

is

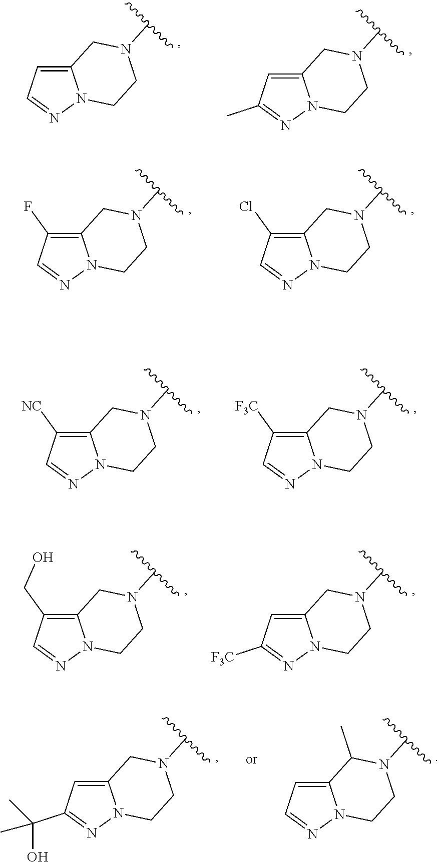

In some embodiments of a compound of Formula (I), (I-1), (Ia)-(Ie), or (Ia-1)-(Ie-1),

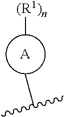

is

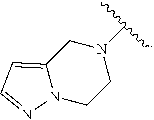 [SMILES: *N1CCn2nccc2C1]

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, —NH₂, —NHCH₃, —N(CH₃)₂, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl; or two R on the same atom form an oxo. In some embodiments of a compound disclosed herein, each R is independently halogen, —CN, —OH, C₁-C₆alkyl, C₁-C₆alkoxy, or C₁-C₆haloalkyl; or two R on the same atom form an oxo. In some embodiments of a compound disclosed herein, each R is independently halogen, —CN, —OH, or C₁-C₆alkyl; or two R on the same atom form an oxo. In some embodiments of a compound disclosed herein, each R is independently halogen, —OH, or C₁-C₆alkyl. In some embodiments of a compound disclosed herein, each R is independently halogen or C₁-C₆alkyl. In some embodiments of a compound disclosed herein, each R is independently halogen.

In some embodiments of a compound disclosed herein, one or more of R, R¹, R^{1a}, R², R³, R⁴, R⁵, R⁶, R^{X}, R^{Xa}, R^{Y}, R^{Ya}, R^{Z}, R_{W}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R^{1a}, R², R³, R⁴, R⁵, R⁶, R^{X}, R^{Xa}, R^{Y}, R^{Ya}, R^{Z}, R^{W}, R^{a}, R^{b}, R^{c}, and R^{d}.

In some embodiments of a compound disclosed herein, the abundance of deuterium in each of R, R¹, R^{1a}, R², R³, R⁴, R⁵, R⁶, R^{X}, R^{Xa}, R^{Y}, R^{Ya}, R^{Z}, R^{W}, R^{a}, R^{b}, R^{c}, and R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more hydrogens of Ring A, Ring B, Ring C, or Ring D are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| Example | Structure |
|---|---|
| 1 | 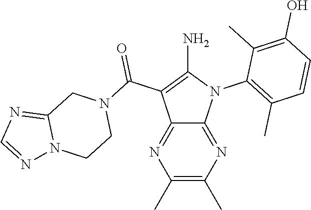 [SMILES: Cc1ccc(O)c(C)c1-n1c(N)c(C(=O)N2CCn3ncnc3C2)c2nc(C)c(C)nc21] |
| 2 | 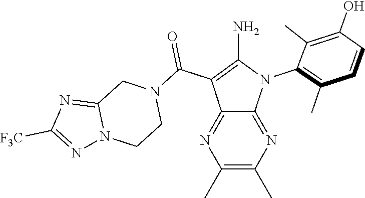 |
| 3 | 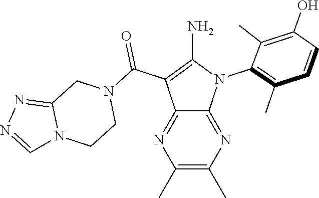 |
| 4 | 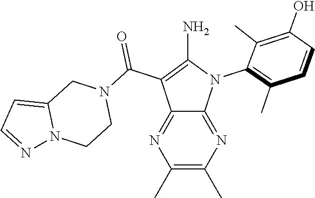 |
| 5 | 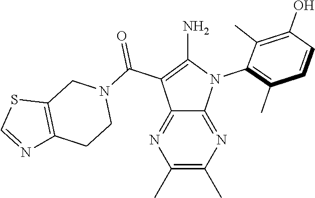 |
| 6 | 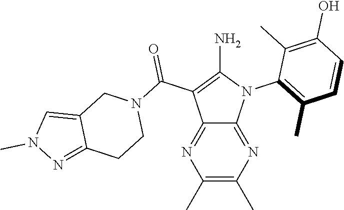 |
| 7 | 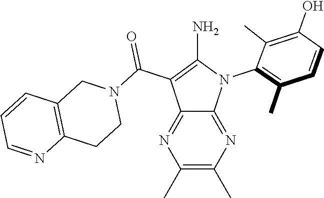 |
| 8 | 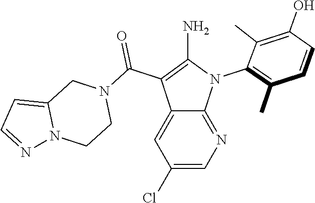 |
| 9 | 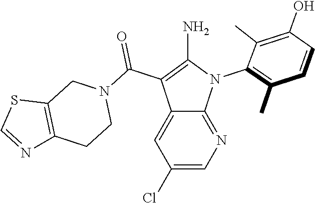 |
| 10 | 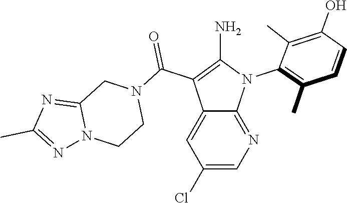 |
| 11 | 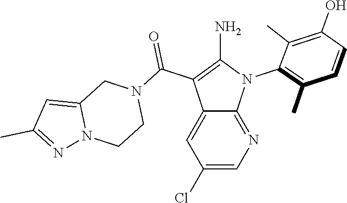 |
| 12 | 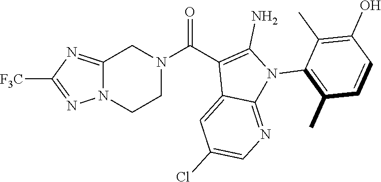 |
| 13 | 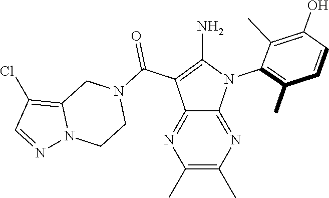 |
| 14 | 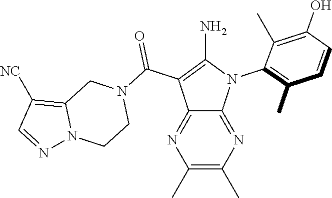 |
| 15 | 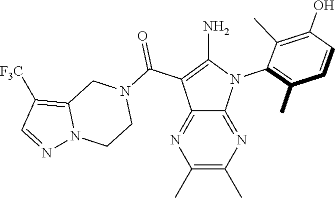 |
| 16 | 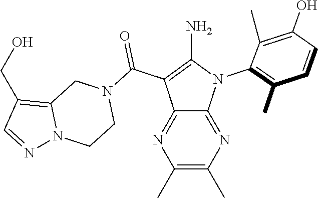 |
| 17 | 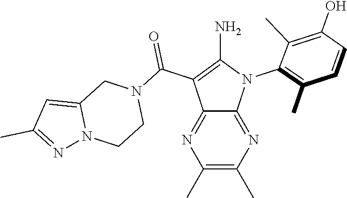 |
| 18 | 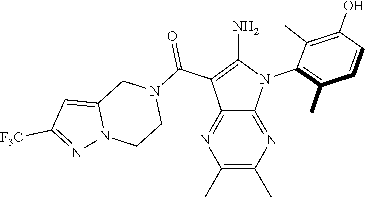 |
| 19 | 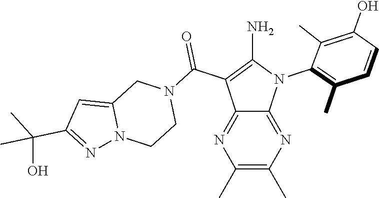 |
| 20 | 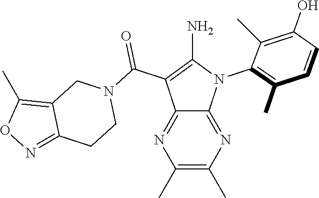 |
| 21 | 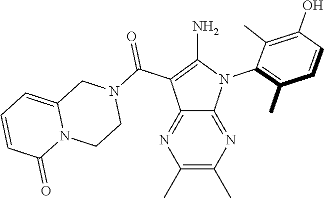 |
| 22 | 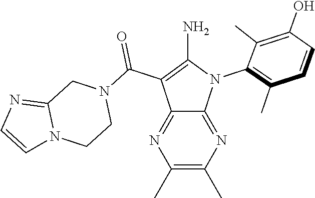 |
| 23 | 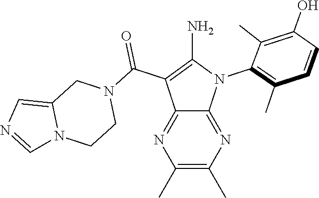 |
| 24 | 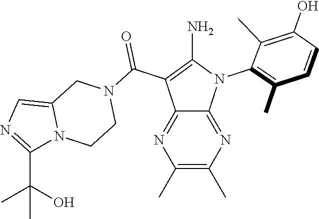 |
| 25 | 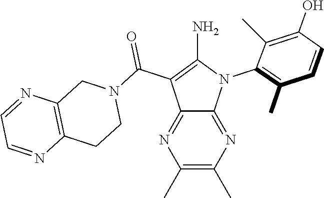 |
| 26 | 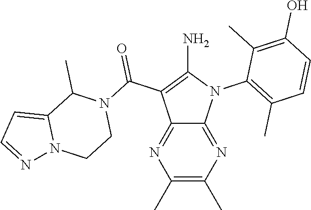 [SMILES: Cc1ccc(O)c(C)c1-n1c(N)c(C(=O)N2CCn3nccc3C2C)c2nc(C)c(C)nc21] |
| 27 | 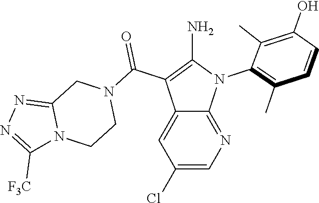 |
| 28 | 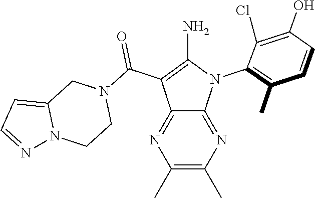 |
| 29 | 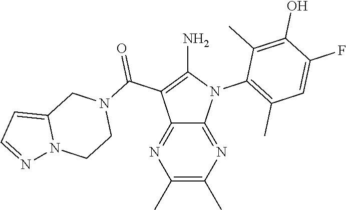 [SMILES: Cc1cc(F)c(O)c(C)c1-n1c(N)c(C(=O)N2CCn3nccc3C2)c2nc(C)c(C)nc21] |
| 30 | 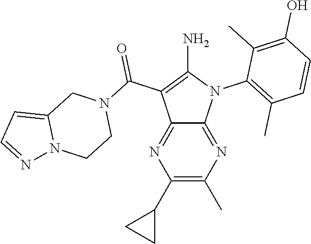 [SMILES: Cc1ccc(O)c(C)c1-n1c(N)c(C(=O)N2CCn3nccc3C2)c2nc(C3CC3)c(C)nc21] |
| 31 | 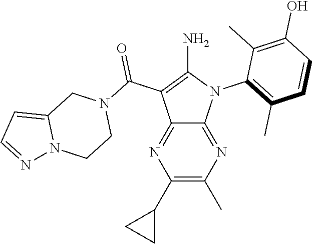 |
| 32 | 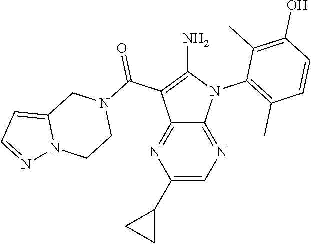 [SMILES: Cc1ccc(O)c(C)c1-n1c(N)c(C(=O)N2CCn3nccc3C2)c2nc(C3CC3)cnc21] |
| 33 | 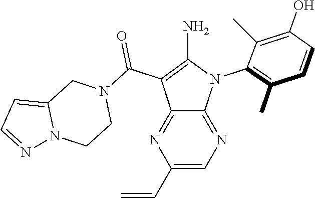 |
| 34 | 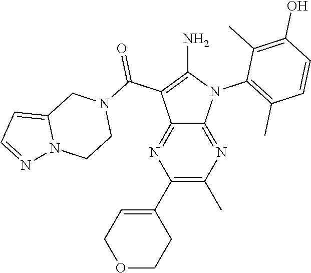 [SMILES: Cc1ccc(O)c(C)c1-n1c(N)c(C(=O)N2CCn3nccc3C2)c2nc(C3=CCOCC3)c(C)nc21] |
| 35 | 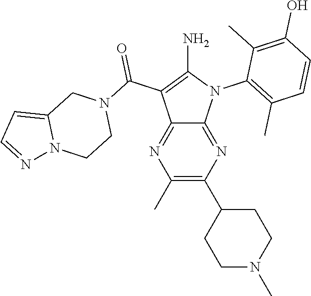 [SMILES: Cc1ccc(O)c(C)c1-n1c(N)c(C(=O)N2CCn3nccc3C2)c2nc(C)c(C3CCN(C)CC3)nc21] |
| 36 | 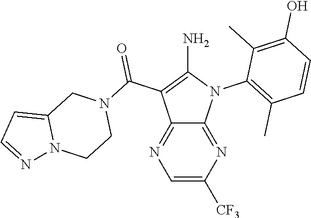 [SMILES: Cc1ccc(O)c(C)c1-n1c(N)c(C(=O)N2CCn3nccc3C2)c2ncc(C(F)(F)F)nc21] |
| 37 | 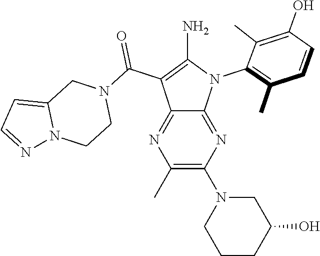 |
| 38 | 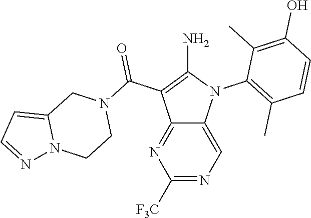 [SMILES: Cc1ccc(O)c(C)c1-n1c(N)c(C(=O)N2CCn3nccc3C2)c2nc(C(F)(F)F)ncc21] |
| 39 | 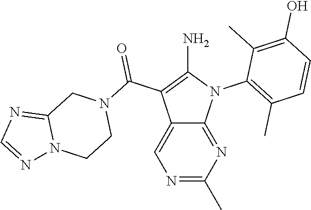 [SMILES: Cc1ncc2c(C(=O)N3CCn4ncnc4C3)c(N)n(-c3c(C)ccc(O)c3C)c2n1] |
| 40 | 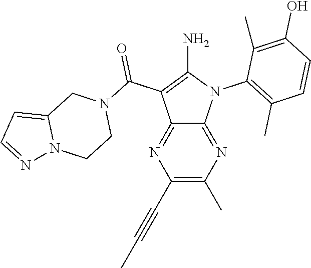 [SMILES: CC#Cc1nc2c(C(=O)N3CCn4nccc4C3)c(N)n(-c3c(C)ccc(O)c3C)c2nc1C] |

In some embodiments the compound disclosed herein, or a pharmaceutically acceptable salt thereof, is one of the compounds in Table 2.

TABLE 2
Structure

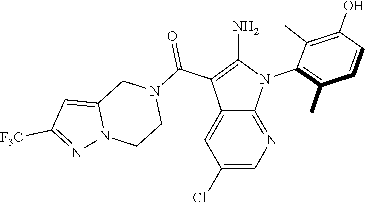

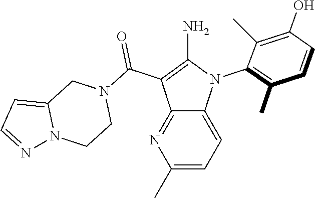

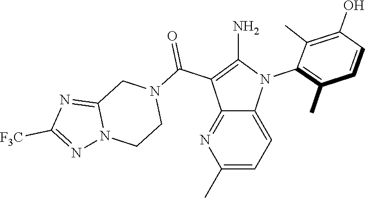

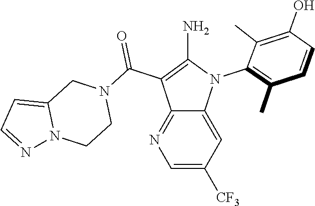

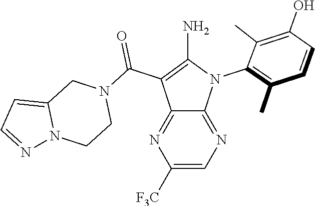

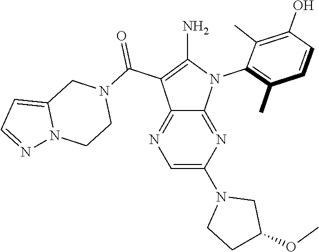

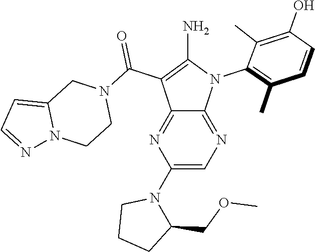

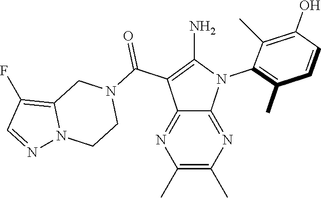

### Further Forms of Compounds Disclosed Herein Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility.

In some embodiments, the optically pure enantiomer is then recovered, along with the resolving agent, by any practical means that would not result in racemization.

In some embodiments, the compounds described herein contain bonds with hindered rotation such that two separate rotamers or atropisomers can be isolated. In some embodiments the atropisomers are

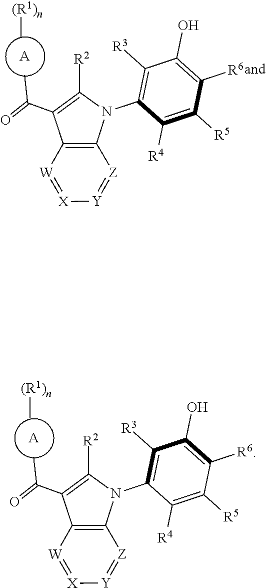

In some embodiments, these atropisomers are separated and are found to have different biological activity which may be advantageous. In some embodiments the atropisomer is

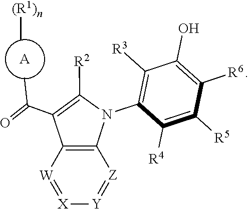

In some embodiments the atropisomer is

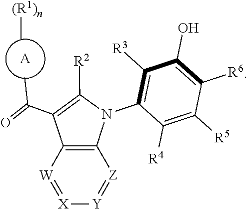

In some embodiments the compound disclosed herein is a racemic mixture:

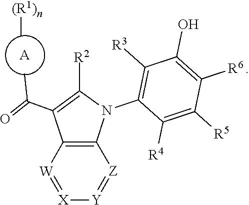

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ³C, ¹⁴C, ¹⁵N, ¹⁸O, ¹⁷O, ³¹P, ³²P, ³⁵S, ⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treating a disease modulated at least in part by PKMYT1 in a subject in need thereof, comprising administering to the subject a therapeutically affective amount of a compound, or a pharmaceutically acceptable salt thereof, disclosed herein.

Disclosed herein is a method of treating cancer in a subject in need thereof, the method comprising administering to the subject a therapeutically affective amount of a compound, or a pharmaceutically acceptable salt thereof, disclosed herein.

In some embodiments, the cancer depends on the activity of PKMYT1.

In some embodiments, the cancer overexpresses CCNE1. In some embodiments cancers which have a high incidence of CCNE1 overexpression include e.g., breast cancer, endometrial cancer, esophageal cancer, lung cancer, ovarian cancer, stomach cancer, and uterine cancer.

In some embodiments, the cancer has an inactivating mutation in the FBXW7 gene. In some embodiments cancers which have a deficiency in FBXW7 include, e.g., breast cancer, colorectal cancer, esophageal cancer, lung cancer, and uterine cancer.

In some embodiments, the cancer is a solid tumor.

In some embodiments, the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.

Disclosed herein is a method of modulating PKMYT1 in a subject, the method comprising administering to the subject a compound, or a pharmaceutically acceptable salt thereof, disclosed herein. Disclosed herein is a method of inhibiting PKMYT1 in a subject, the method comprising administering to the subject a compound, or a pharmaceutically acceptable salt thereof, disclosed herein.

Further disclosed herein is a method of selectively inhibiting PKMYT1 in a subject (e.g., selective over WEE1), the method comprising administering to the subject a compound, or a pharmaceutically acceptable salt thereof, disclosed herein.

WEE1 widely exists in human tissues and plays an important role in all phases of the cell cycle. It can be beneficial to use compounds that selectively inhibit PKMYT1 over WEE1 in methods of treatment described herein. In some embodiments, compounds disclosed herein, or a pharmaceutically acceptable salt thereof, do not interact with WEE1. In some embodiments, compounds disclosed herein, or a pharmaceutically acceptable salt thereof, do not inhibit WEE1. In some embodiments, a compound disclosed herein has a WEE1 IC₅₀ value of at least about 100 nM, at least about 500 nM, at least about 1000 nM, or at least about 10,000 nM as determined in a WEE1 ADP-Glo assay. For example, the WEE1 IC₅₀ value can be determined in a WEE1 ADP-Glo assay. In some embodiments, the compound disclosed herein, or a pharmaceutically acceptable salt thereof, has a WEE1 IC₅₀ value of at least about 10,000 nM as determined in a WEE1 ADP-Glo assay. In some embodiments, the compound disclosed herein, or a pharmaceutically acceptable salt thereof, has a WEE1 IC₅₀ value of at least about 5,000 nM as determined in a WEE1 ADP-Glo assay. In some embodiments, the compound disclosed herein, or a pharmaceutically acceptable salt thereof, has a WEE1 IC₅₀ value of at least about 4,000 nM as determined in a WEE1 ADP-Glo assay. In some embodiments, the compound disclosed herein, or a pharmaceutically acceptable salt thereof, has a WEE1 IC₅₀ value of at least about 3,000 nM as determined in a WEE1 ADP-Glo assay. In some embodiments, the compound disclosed herein, or a pharmaceutically acceptable salt thereof, has a WEE1 IC₅₀ value of at least about 2,000 nM as determined in a WEE1 ADP-Glo assay. In some embodiments, the compound disclosed herein, or a pharmaceutically acceptable salt thereof, has a WEE1 IC₅₀ value of at least about 1,000 nM as determined in a WEE1 ADP-Glo assay.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating a disease or disorder associated with PKMYT1 using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent.

### EXAMPLES

### Intermediate 1: Preparation of ethyl 6-amino-5-(3-methoxy-2,6-dimethylphenyl)-2,3-dimethyl-5H-pyrrolo[2,3-b]pyrazine-7-carboxylate

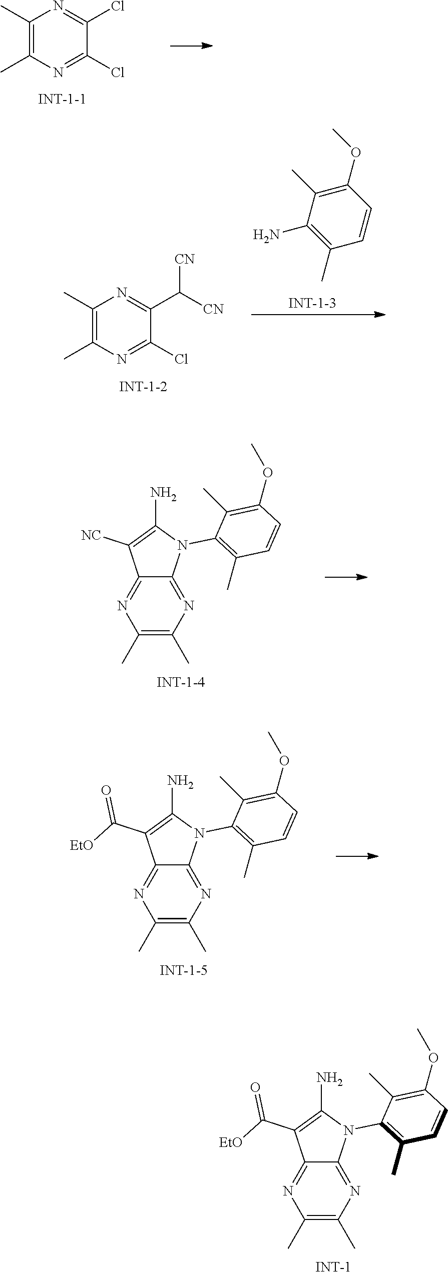

### Step 1: Preparation of Compound INT-1-2

To a solution of propanedinitrile (7.46 g, 112.98 mmol) in THF (200 mL) was added NaH (6.78 g, 169.46 mmol). The mixture was stirred at 0° C. under N₂ for 0.5 h. Then compound INT-1-1 (10 g, 56.48 mmol) and Pd(PPh₃)₄ (3.26 g, 2.824 mmol) were added. The mixture was stirred at 75° C. under N₂ for 4 hours. The reaction was quenched by H₂O (100 mL), and neutralized with HCl (1M in water). The mixture was extracted with EA (500 mL×3), washed with brine (300 mL), dried over anhydrous sodium sulfate, filtered, and concentrated to dryness. The residue was purified by column chromatography on silica gel to afford compound INT-1-2 (12 g, quantitively) as a yellow solid.

### Step 2: Preparation of Compound INT-1-4

To a solution of compound INT-1-2 (10 g, 48.40 mmol) in NMP (100 mL) was added compound INT-1-3 (14.64 g, 96.79 mmol), t-BuOK (16.26 g, 145.187 mmol) and Pd-PEPPSI(TM)-IPent catalyst (1.65 g, 2.42 mmol). The mixture was stirred at 110° C. under N₂ for 1 h. The reaction was quenched by ice water (200 mL), extracted with EA (200 mL×3), washed with brine (100 mL), dried over anhydrous sodium sulfate, and concentrated. The residue was purified by column chromatography on silica gel to afford compound INT-1-4 (10 g, 64.3%) as a yellow solid. LCMS: 322.1 [M+H]⁺.

### Step 3: Preparation of ethyl 6-amino-5-(3-methoxy-2,6-dimethylphenyl)-2,3-dimethyl-5H-pyrrolo[2,3-b]pyrazine-7-carboxylate

To a solution of compound INT-1-4 (3.0 g, 9.33 mmol) in EtOH (25 mL) was added sulfuric acid (5 mL). The mixture was stirred at 100° C. under N₂ for 16 hours. The reaction was quenched by ice-water (100 mL), basified to pH-8 with NaOH (4M in water), extracted with EA (100 mL*3). The organic layer was dried over anhydrous sodium sulfate, filtered, and concentrated to dryness. The residue was purified by column chromatography on silica gel to afford compound INT-1-5 (1.2 g, 34.89%) as a yellow solid. The enantiomers of compound INT-1-5 were purified by chiral preparative HPLC to afford the common intermediate INT-1. LCMS: 369.1 [M+H]⁺.

### Intermediate 2: Preparation of methyl 2-amino-5-chloro-1-(3-hydroxy-2,6-dimethylphenyl)-1H-pyrrolo[2,3-b]pyridine-3-carboxylate

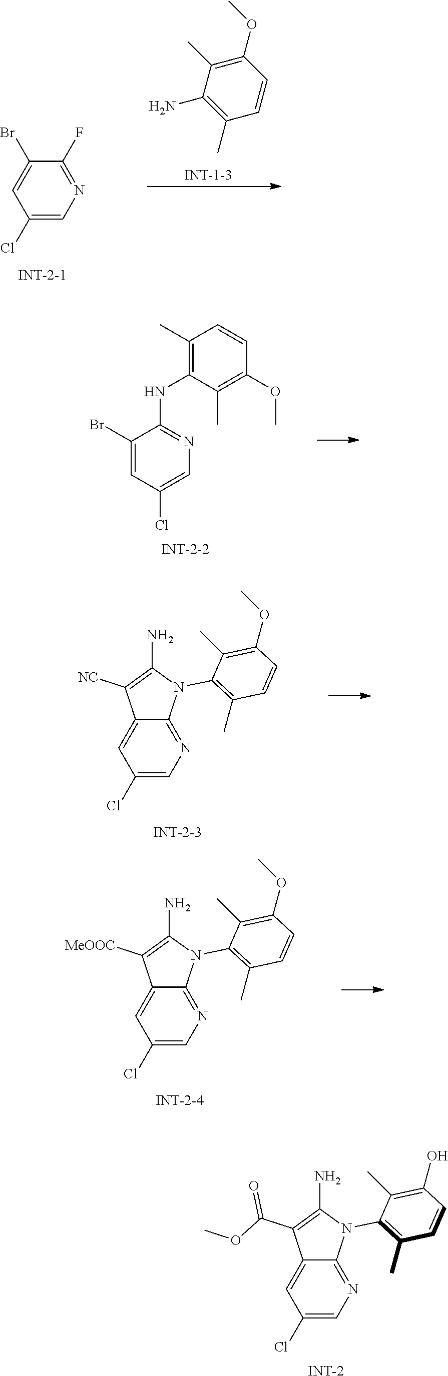

### Step 1: Preparation of Compound INT-2-2

To a solution of compound INT-2-1 (2.2 g, 10.45 mmol), compound INT-1-3 (1.58 g, 10.45 mmol) in THE (20 mL) was added LiHMDS (10.5 mL) at 0° C. The mixture was stirred at room temperature for 2 hours, and quenched with water (200 mL). The resulting mixture was extracted with EA (3×200 mL). The EA layers were combined, washed with brine (2×200 mL), dried with Na₂SO₄, filtered and concentrated. The residue was purified by silica gel column chromatography to afford compound INT-2-2 (3.2 g, 89.6%) as a yellow solid. LCMS: 342.9 [M+H]⁺.

### Step 2: Preparation of Compound INT-2-3

To a solution of propanedinitrile (0.60 mL, 9.37 mmol) in DME (30 mL) were added NaH (0.22 g, 9.37 mmol) at 0° C., the reaction was stirred at room temperature for 30 min. Then compound INT-2-2 (3.2 g, 9.37 mmol) was added. The mixture was stirred at 100° C. for 5 hours, and quenched with water (200 mL). The resulting mixture was extracted with EA (3×200 mL). The EA layers were combined and washed with brine (2×200 mL), dried by Na₂SO₄, filtered and concentrated. The residue was purified by silica gel column chromatography to afford compound INT-2-3 (2.7 g, 88.2%) as a yellow solid. LCMS: 327.1 [M+H]⁺.

### Step 3: Preparation of methyl 2-amino-5-chloro-1-(3-hydroxy-2,6-dimethylphenyl)-1H-pyrrolo[2,3-b]pyridine-3-carboxylate

To a stirred solution of compound INT-2-3 (3.0 g, 9.20 mmol) in MeOH (30 mL) was added conc. sulfuric acid (6 mL). The reaction mixture was stirred at 85° C. for 16 hours. The reaction was quenched by ice-water (100 mL). The mixture was adjusted to pH ˜8 with NaOH (4 M aq.), and extracted with EA (100 mL×3). The organic layer was dried over anhydrous sodium sulfate, filtered, and concentrated to dryness. The residue was purified by column chromatography on silica gel to afford compound INT-2-4 (2.47 g, 67.2%) as a yellow solid. The enantiomers of compound INT-2-4 were purified by chiral preparative HPLC to afford the common intermediate INT-2. LCMS: 360.1 [M+H]⁺.

### Example 1: Preparation of (6-amino-5-(3-hydroxy-2,6-dimethylphenyl)-2,3-dimethyl-5H-pyrrolo[2,3-b]pyrazin-7-yl)(5,6-dihydro-[1,2,4]triazolo[1,5-a]pyrazin-7(8H)-yl)methanone

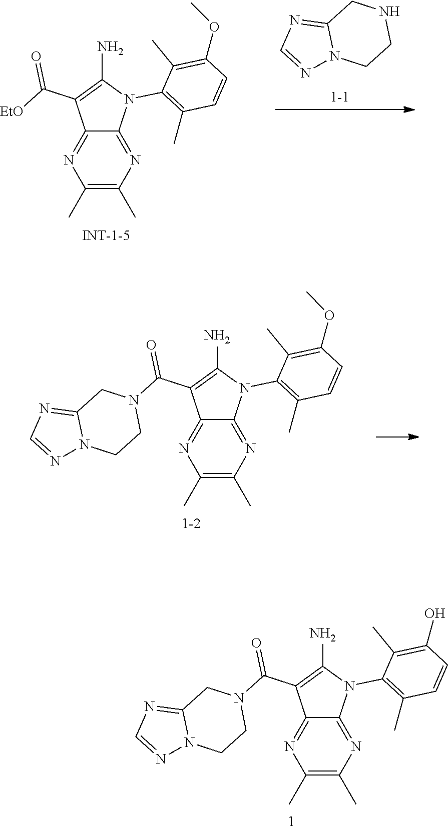

### Step 1: Preparation of Compound 1-2

To a solution of compound INT-1-5 (50 mg, 0.136 mmol) in dioxane (1 mL) was added compound 1-1 (20.2 mg, 0.163 mmol) and trimethylaluminum (0.68 mL, 1 M in hexane, 0.679 mmol). The reaction mixture was stirred at 90° C. for 30 min. The reaction was quenched by MeOH (10 mL), and filtered. The filtrate was concentrated to dryness to afford compound 1-2 as a brown solid. LCMS: 447.2 [M+H]⁺.

### Step 2: Preparation of Example 1

To a solution of compound 1-2 (50 mg, 0.112 mmol) in DCM (10 mL) was added BBr₃ (28.1 mg, 0.112 mmol). The reaction mixture was stirred at 20° C. for 1 h under nitrogen. The reaction was quenched by MeOH (10 mL), and filtered. The filtrate was concentrated to dryness, and purified by Pre-HPLC to afford example 1 as a white solid. LCMS: 433.2 [M+H]⁺. ¹H NMR: (400 MHz, DMSO-d₆) δ 9.59 (s, 1H), 7.99 (s, 1H), 7.08 (d, J=7.8 Hz, 2H), 7.07 (s, 1H), 6.93 (d, J=8.3 Hz, 1H), 4.91 (s, 2H), 4.46 (s, 2H), 4.17 (d, J=5.0 Hz, 2H), 2.48 (m, 3H), 2.32 (s, 3H), 1.77 (s, 3H), 1.69 (s, 3H).

### Example 2: Preparation of (6-amino-5-(3-hydroxy-2,6-dimethylphenyl)-2,3-dimethyl-5H-pyrrolo[2,3-b]pyrazin-7-yl)(2-(trifluoromethyl)-5,6-dihydro-[1,2,4]triazolo[1,5-a]pyrazin-7(8H)-yl)methanone

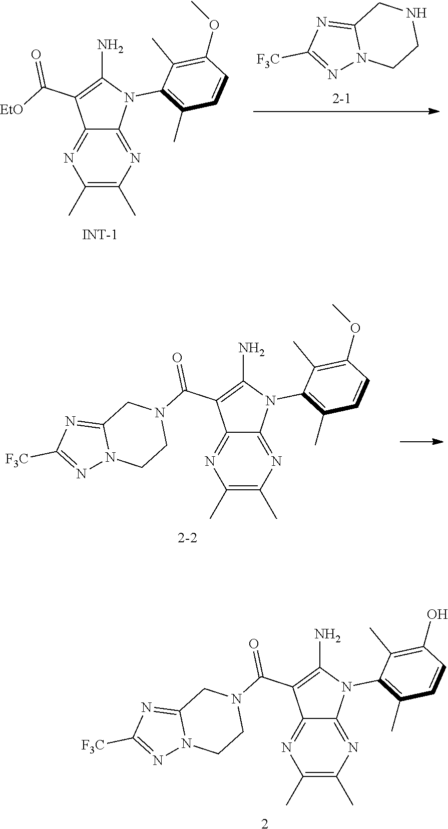

### Step 1: Preparation of Compound 2-2

To a solution of compound INT-1 (50 mg, 0.136 mmol) in dioxane (2 mL) was added compound 2-1 (31.29 mg, 0.163 mmol) and Me₃Al (1.36 mL, 1M in hexane). The mixture was stirred at 120° C. under N₂ for 1 h. The reaction was quenched by MeOH (10 mL), and filtered. The filtrate was concentrated to dryness to afford compound 2-2 as a yellow solid. LCMS: 446.2 [M+H]⁺.

### Step 1: Preparation of Example 2

To a solution of compound 2-2 (50 mg, 0.100 mmol) in DCM (3 mL) was added BBr₃ (0.5 mL). The mixture was stirred at 20° C. under N₂ for 1 h. The reaction was quenched by MeOH (10 mL), and filtered. The filtrate was concentrated to dryness. The residue was purified by Pre-HPLC to afford example 2 as a yellow solid. LCMS: 500.9 [M+H]⁺. ¹H NMR: (400 MHz, DMSO-d₆) δ 9.58 (s, 1H), 7.21-6.87 (m, 4H), 4.99 (s, 2H), 4.57 (s, 2H), 4.21 (s, 2H), 2.47 (s, 3H), 2.32 (s, 3H), 1.78 (s, 3H), 1.69 (s, 3H). ¹⁹F NMR (376 MHz, DMSO-d₆) δ −63.98 (s).

### Example 3: Preparation of (6-amino-5-(3-hydroxy-2,6-dimethylphenyl)-2,3-dimethyl-5H-pyrrolo[2,3-b]pyrazin-7-yl)(5,6-dihydro-[1,2,4]triazolo[4,3-a]pyrazin-7(8H)-yl)methanone

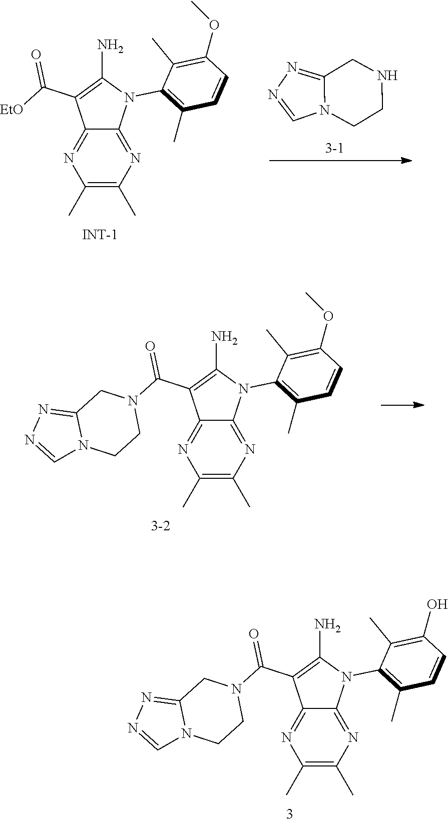

### Step 1: Preparation of Compound 3-2

To a solution of compound INT-1 (50 mg, 0.136 mmol) in dioxane (2 mL) was added compound 3-1 (31.29 mg, 0.163 mmol) and Me₃Al (1.36 mL, 1 M in hexane). The mixture was stirred at 120 T under N₂ for 1 h. The reaction was quenched by MeOH (10 mL), and filtered. The filtrate was concentrated to dryness to afford compound 3-2 as a yellow solid. LCMS: 447.1 [M+H]⁺.

### Step 2: Preparation of Example 3

To a solution of compound 3-2 (50 mg, 0.112 mmol) in DCM (3 mL) was added BBr₃ (0.5 mL). The mixture was stirred at 20° C. under N₂ for 1 h. The reaction was quenched by addition of MeOH (10 mL), which was filtered, the filtrate was concentrated to dryness. The residue was purified by Pre-HPLC to afford example 3 as a yellow solid. LCMS: 432.2 [M+H]⁺. ¹H NMR: (400 MHz, DMSO-d₆) δ 9.58 (s, 1H), 8.56 (s, 1H), 7.14-7.03 (m, 3H), 6.94 (t, J=6.2 Hz, 1H), 4.94 (s, 2H), 4.39 (s, 2H), 4.06 (s, 2H), 2.48 (s, 3H), 2.32 (s, 3H), 1.77 (s, 3H), 1.69 (s, 3H).

### Example 4: Preparation of (6-amino-5-(3-hydroxy-2,6-dimethylphenyl)-2,3-dimethyl-5H-pyrrolo[2,3-b]pyrazin-7-yl)(6,7-dihydropyrazolo[1,5-a]pyrazin-5(4H)-yl)methanone

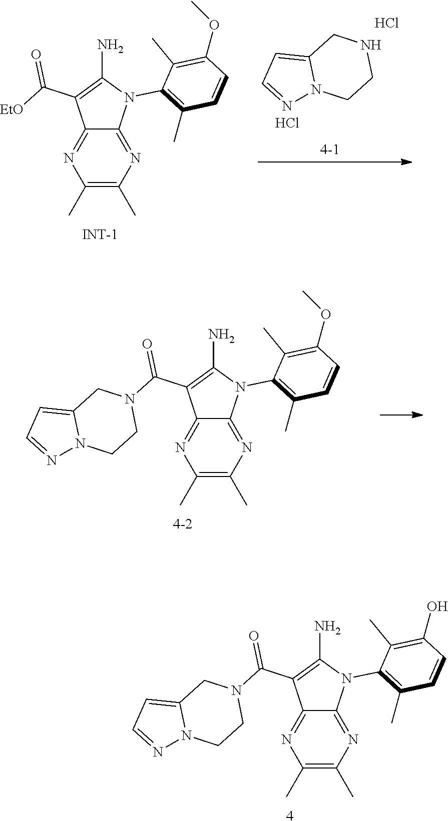

### Step 1: Preparation of Compound 4-2

To a solution of compound INT-1 (50 mg, 0.136 mmol) in dioxane (2 mL) was added compound 4-1 (20.06 mg, 0.163 mmol) and Me₃Al (1.36 mL, 1 M in hexane). The mixture was stirred at 120° C. under N₂ for 1 h. The reaction was quenched by addition of MeOH (10 mL), and filtered. The filtrate was concentrated to dryness to afford compound 4-2 as a yellow solid. LCMS: 446.1 [M+H]⁺.

### Step 2: Preparation of Example 4

To a solution of compound 4-2 (50 mg, 0.112 mmol) in DCM (3 mL) was added BBr₃ (0.5 mL). The mixture was stirred at 20° C. under N₂ for 1 h. The reaction was quenched by addition of MeOH (10 mL), and filtered. The filtrate was concentrated to dryness. The residue was purified by Pre-HPLC to afford example 4 as a yellow solid. LCMS: 432.2 [M+H]⁺. ¹H NMR: (400 MHz, DMSO-d₆) δ 9.61 (s, 1H), 8.38 (s, 1H), 7.45 (s, 1H), 7.13-6.86 (m, 4H), 6.15 (s, 1H), 4.85 (s, 2H), 4.41 (s, 2H), 4.11 (s, 2H), 2.47 (s, 3H), 2.31 (s, 3H), 1.77 (s, 3H), 1.69 (s, 3H).

### Example 5: Preparation of (6-amino-5-(3-hydroxy-2,6-dimethylphenyl)-2,3-dimethyl-5H-pyrrolo[2,3-b]pyrazin-7-yl)(6,7-dihydrothiazolo[5,4-c]pyridin-5(4H)-yl)methanone

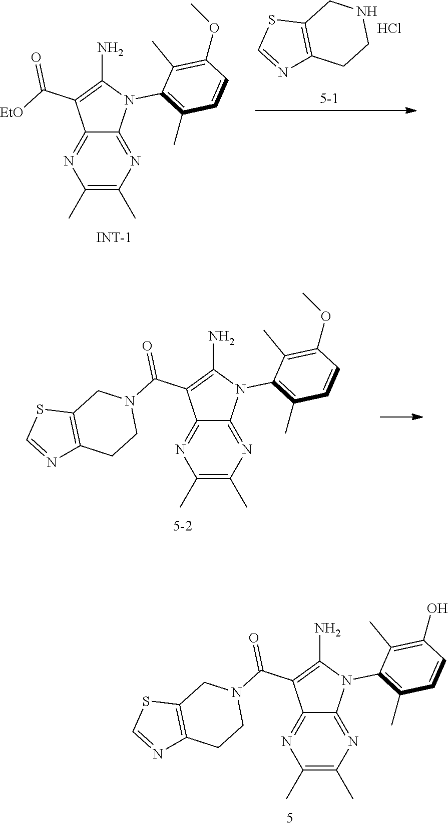

### Step 1: Preparation of Compound 5-2

To a solution of compound INT-1 (25 mg, 0.068 mmol) and compound 5-1 (15 mg, 0.082 mmol) in dioxane (1 mL) was added TEA (21 mg, 0.204 mmol) and AlMe₃ (0.34 ml, 0.34 mmol). The mixture was stirred at 90° C. for 3 h. MeOH (10 mL) was added to quench the reaction. The precipitation was filtered from the solution. The filtrate was concentrated, and dried to afford compound 5-2 as a white crude solid. LCMS: 463.4 [M+H]⁺.

### Step 2: Preparation of Example 5

To a solution of compound 5-2 (40 mg, crude) in DCM (3 mL) was added BBr₃ (0.1 mL). The mixture was stirred at 20 T for 30 mins. The mixture was concentrated. The crude product was purified by Pre-HPLC to give example 5 as a white solid. LCMS: 449.3 [M+H]⁺. ¹H NMR: (400 MHz, DMSO-d₆) δ 9.00 (s, 1H), 8.45 (s, 2H), 7.08 (d, J=8.5 Hz, 1H), 6.95 (s, 3H), 4.91 (s, 2H), 3.97 (s, 2H), 3.14 (s, 2H), 2.48 (s, 3H), 2.32 (s, 3H), 1.78 (s, 3H), 1.69 (s, 3H).

### Example 6: Preparation of (6-amino-5-(3-hydroxy-2,6-dimethylphenyl)-2,3-dimethyl-5H-pyrrolo[2,3-b]pyrazin-7-yl)(2-methyl-2,4,6,7-tetrahydro-5H-pyrazolo[4,3-c]pyridin-5-yl)methanone

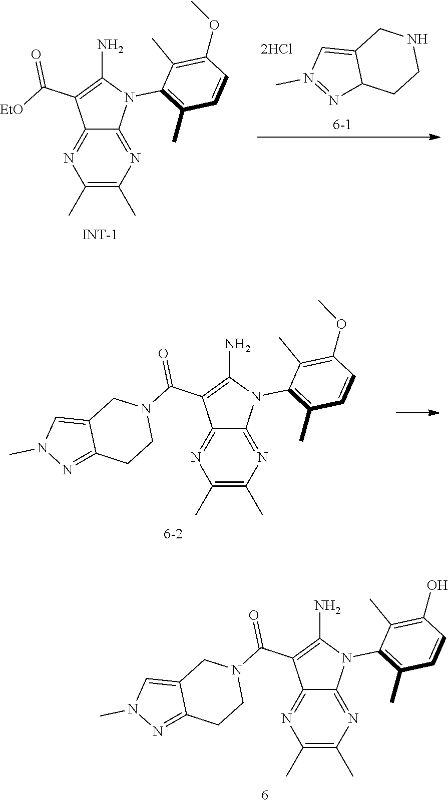

### Step 1: Preparation of Compound 6-2

To a solution of compound INT-1 (50 mg, 0.136 mmol) in dioxane (2 mL) was added compound 6-1 (31 mg, 0.163 mmol) and Me₃Al (1.36 mL, 1M in hexane). The mixture was stirred at 120° C. under N₂ for 1 h. The reaction was quenched by addition of MeOH (10 mL). The mixture was filtered, the filtrate was concentrated to dryness to afford compound 6-2 as a yellow solid, which was determined by LCMS. LCMS: 460.0 [M+H]⁺.

### Step 2: Preparation of Example 6

To a solution of compound 6-2 (50 mg, 0.112 mmol) in DCM (3 mL) was added BBr₃ (0.5 mL). The mixture was reacted at 20° C. under N₂ for 1 h. The reaction was quenched by addition of MeOH (10 mL). The mixture was filtered, the filtrate was concentrated to dryness. The residue was purified by Pre-HPLC to afford example 6 as a yellow solid. LCMS: 446.2 [M+H]⁺. ¹H NMR: (400 MHz, DMSO-d₆) δ 9.65 (s, 1H), 7.46 (s, 1H), 7.07 (d, J=8.2 Hz, 1H), 6.98-6.81 (m, 3H), 4.60 (s, 2H), 3.91-3.80 (m, 2H), 3.76 (s, 3H), 3.02-2.84 (m, 2H), 2.47 (s, 3H), 2.31 (s, 3H), 1.77 (s, 3H), 1.68 (s, 3H).

### Example 7: Preparation of (6-amino-5-(3-hydroxy-2,6-dimethylphenyl)-2,3-dimethyl-5H-pyrrolo[2,3-b]pyrazin-7-yl)(7,8-dihydro-1,6-naphthyridin-6(5H)-yl)methanone

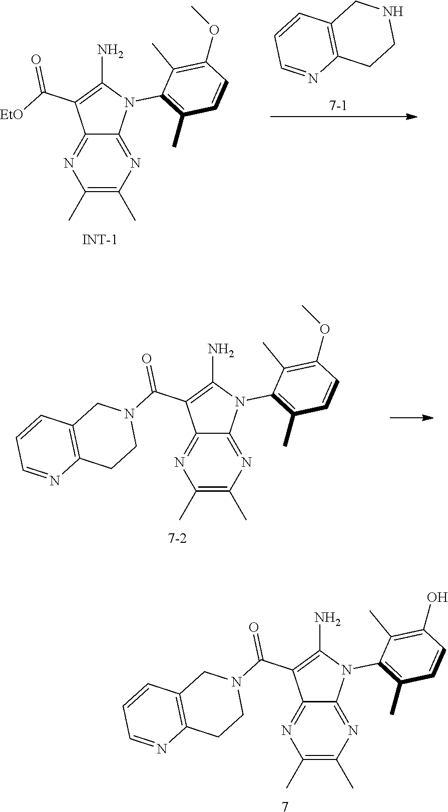

### Step 1: Preparation of Compound 7-2

To a solution of compound INT-1 (30 mg, 0.081 mmol) and compound 7-1 (13.1 mg, 0.098 mmol) in dioxane (2 mL) was added trimethyl aluminum (0.8 mL, 1M in toluene) dropwise. The mixture was stirred at 90 T for 1 h. MeOH (5 mL) was added to quench the reaction. The mixture was filtered and the filtrate was concentrated to afford compound 7-2 as a yellow solid and used directly in the next step without further purification. LCMS: 457.3 [M+H]⁺.

### Step 2: Preparation of Example 7

To a solution of compound 7-2 (37.2 mg, 0.081 mmol) in DCM (2 mL) was added BBr₃ (0.1 mL, 1 M in DCM). The mixture was stirred at 20 T for 1 h. MeOH (5 mL) was added. The mixture was concentrated to remove the solvent. The crude product was purified by prep-HPLC to afford example 7 as a white solid. LCMS: 443.2 [M+H]⁺. ¹H NMR: (400 MHz, DMSO-d₆) δ 9.60 (s, 1H), 8.41-8.39 (m, 1H), 7.63 (d, J=7.6 Hz, 11H), 7.22 (dd, J=7.7, 4.8 Hz, 11H), 7.07 (d, J=8.3 Hz, 1H), 6.93 (d, J=8.1 Hz, 3H), 4.82 (s, 2H), 3.97 (t, J=5.7 Hz, 2H), 3.21 (s, 2H), 2.46 (s, 3H), 2.31 (s, 3H), 1.78 (s, 3H), 1.69 (s, 3H).

### Example 8: Preparation of (2-amino-5-chloro-1-(3-hydroxy-2,6-dimethylphenyl)-1H-pyrrolo[2,3-b]pyridin-3-yl)(6,7-dihydropyrazolo[1,5-a]pyrazin-5(4H)-yl)methanone

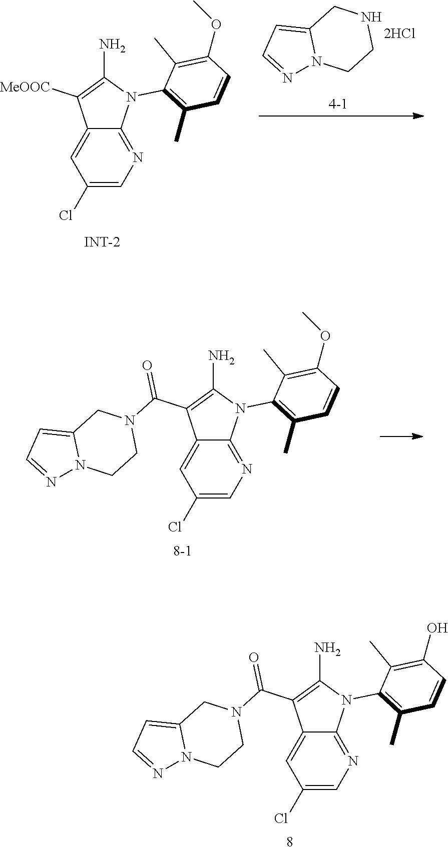

### Step 1: Preparation of Compound 8-1

To a solution of compound 4-1 (40 mg, 0.11 mmol) in dioxane (3 mL) was added DIPEA (0.19 mL), Me₃Al (1.1 mL, 1 M in toluene). The mixture was stirred at 15° C. under N₂ for 1 h. Then compound INT-2 (40 mg, 0.11 mmol) was added. The mixture was stirred at 120 T under N₂ for 24 h. The reaction mixture was quenched by MeOH (10 mL). Then the mixture was filtered. The filtrate was concentrated to dryness and purified by Pre-TLC to afford compound 8-1 as a yellow solid. LCMS: 451.1 [M+H]⁺.

### Step 2: Preparation of Example 8

To a solution of compound 8-1 (30 mg, 0.067 mmol) in DCM (5 mL) was added BBr₃ (0.1 mL). The mixture was stirred at room temperature under N₂ for 1 h. The reaction was quenched by MeOH (5 mL). The mixture was concentrated to dryness. The residue was purified by Pre-HPLC to afford example 8 as a yellow solid. LCMS: 437.4 [M+H]⁺. ¹H NMR: (400 MHz, DMSO-d₆) δ 9.54 (s, 1H), 7.78 (d, J=2.2 Hz, 1H), 7.56 (d, J=2.2 Hz, 1H), 7.43 (d, J=1.7 Hz, 1H), 7.06 (d, J=8.3 Hz, 1H), 6.92 (d, J=8.3 Hz, 1H), 6.64 (s, 2H), 6.13 (d, J=1.5 Hz, 1H), 4.82 (s, 2H), 4.20 (t, J=5.3 Hz, 2H), 3.99 (d, J=5.7 Hz, 2H), 1.76 (s, 3H), 1.68 (s, 3H).

### Example 9: Preparation of (2-amino-5-chloro-1-(3-hydroxy-2,6-dimethylphenyl)-1H-pyrrolo[2,3-b]pyridin-3-yl)(6,7-dihydrothiazolo[5,4-c]pyridin-5(4H)-yl)methanone

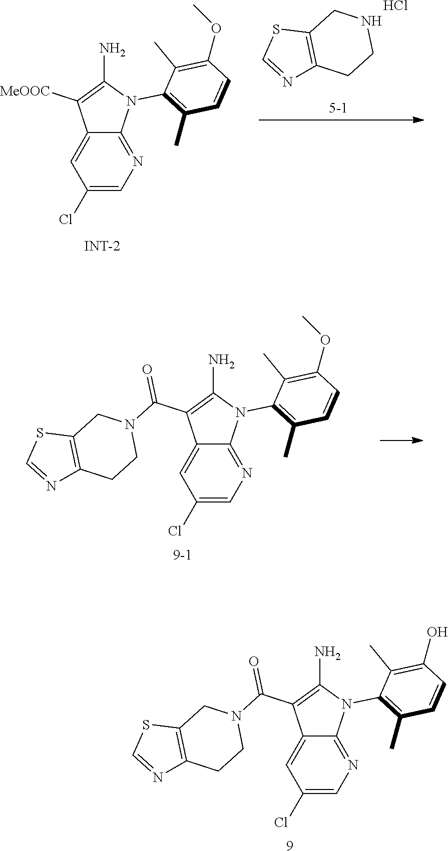

### Step 1: Preparation of Compound 9-1

To a solution of compound 5-1 (147 mg, 0.834 mmol) in dioxane (1 mL) was added trimethyl aluminum (0.63 mL, 1.251 mmol, 2 M in toluene). The mixture was stirred at 15° C. for 1 h. Then compound INT-2 (30 mg, 0.083 mmol) was added. The mixture was heated to 120° C. for 16 h. The reaction mixture was quenched by MeOH (10 mL). The resulting mixture was filtered. The filtrate was concentrated to dryness and purified by Pre-TLC to afford compound 9-1 as a yellow solid. LCMS: 468.1 [M+H]⁺.

### Step 2: Preparation of Example 9

To a solution of compound 9-1 (30 mg, 0.064 mmol) in DCM (5 mL) was added BBr₃ (0.1 mL). The mixture was stirred at room temperature under N₂ for 1 h. The reaction mixture was quenched by MeOH (5 mL). The mixture was concentrated to dryness. The residue was purified by Pre-HPLC to afford example 9 as a yellow solid. LCMS: 454.0 [M+H]⁺. ¹H NMR: (400 MHz, CD₃OD) δ 9.66 (s, 1H), 7.80 (d, J=2.1 Hz, 1H), 7.74 (d, J=2.1 Hz, 1H), 7.13 (d, J=8.3 Hz, 1H), 6.95 (d, J=8.3 Hz, 1H), 5.01 (s, 2H), 4.02 (t, J=5.6 Hz, 2H), 3.13 (dd, J=7.3, 3.8 Hz, 2H), 1.87 (s, 3H), 1.82 (s, 3H).

### Example 10: Preparation of (2-amino-5-chloro-1-(3-hydroxy-2,6-dimethylphenyl)-1H-pyrrolo[2,3-b]pyridin-3-yl)(2-methyl-5,6-dihydro-[1,2,4]triazolo[1,5-a]pyrazin-7(8H)-yl)methanone

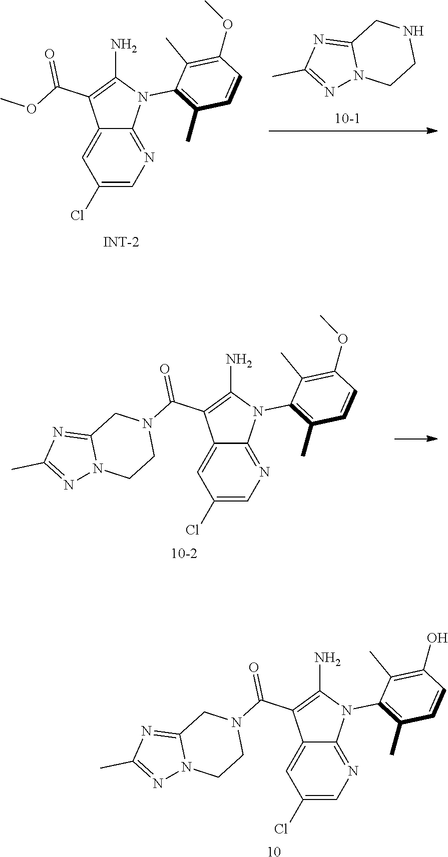

### Step 1: Preparation of Compound 10-2

To a solution of compound 10-1 (114.3 mg, 0.83 mmol) in dioxane (3 mL) was added DIPEA (0.145 mL), Me₃Al (0.42 mL, 2M in toluene). The mixture was stirred at room temperature under N₂ for 1 h. Then compound INT-2 (30 mg, 0.083 mmol) was added. The mixture was stirred at 120 T under N₂ for 24 h. The reaction mixture was quenched by MeOH (10 mL), and filtered. The filtrate was concentrated to dryness and purified by Pre-TLC to afford compound 10-2 as a yellow solid. LCMS: 466.2 [M+H]⁺.

### Step 2: Preparation of Example 10

To a solution of compound 10-2 (30 mg, 0.064 mmol) in DCM (5 mL) was added BBr₃ (0.1 mL). The mixture was stirred at room temperature under N₂ for 1 h. The reaction mixture was quenched by MeOH (5 mL). The mixture was concentrated to dryness. The residue was purified by Pre-HPLC to afford example 10 as a yellow solid. LCMS: 452.2 [M+H]⁺. ¹H NMR: (400 MHz, DMSO-d₆) δ 9.52 (s, 1H), 7.79 (d, J=2.2 Hz, 1H), 7.62 (d, J=2.2 Hz, 1H), 7.07 (d, J=8.3 Hz, 1H), 6.92 (d, J=8.3 Hz, 1H), 6.65 (s, 2H), 4.82 (s, 2H), 4.18 (t, J=5.2 Hz, 2H), 4.00 (d, J=5.7 Hz, 2H), 2.26 (s, 3H), 1.76 (s, 3H), 1.68 (s, 3H).

### Example 11: Preparation of (2-amino-5-chloro-1-(3-hydroxy-2,6-dimethylphenyl)-1H-pyrrolo[2,3-b]pyridin-3-yl)(2-methyl-6,7-dihydropyrazolo[1,5-a]pyrazin-5(4H)-yl)methanone

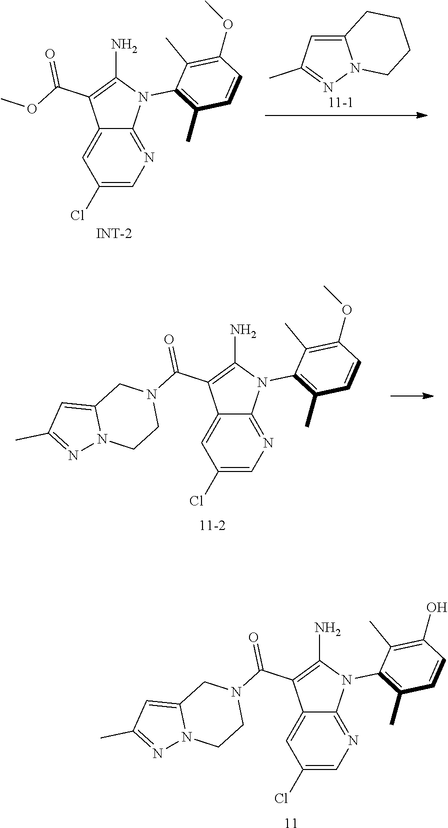

### Step 1: Preparation of Compound 11-2

To a solution of compound INT-2 (30 mg, 0.083 mmol) in dioxane (2 mL) were added compound 11-1 (114 mg, 0.834 mmol), DIPEA (0.21 mL, 1.251 mmol) and Me₃Al (1.25 mL, 1.251 mmol). The mixture was stirred at 90° C. for 24 h. The reaction was quenched by MeOH (10 mL). The resulting mixture was filtered. The filtrate was concentrated, and purified by silica gel column chromatography to afford compound 11-2 as a white solid. LCMS: 465.1 [M+H]⁺.

### Step 2: Preparation of Example 11

To a solution of compound 11-2 (25 mg, 0.054 mmol) in DCM (3 mL) was added BBr₃ (0.1 mL). The reaction mixture was stirred at room temperature for 1 h. The reaction mixture was quenched by MeOH (10 mL), and concentrated to dryness. The crude product was purified by Pre-HPLC to afford example 11 as a white solid. LCMS: 451.2 [M+H]⁺. ¹H NMR (400 MHz, DMSO-d₆) δ 9.52 (s, 1H), 7.78 (d, J=2.2 Hz, 1H), 7.55 (d, J=2.2 Hz, 1H), 7.06 (d, J=8.3 Hz, 1H), 6.92 (d, J=8.3 Hz, 1H), 6.62 (s, 2H), 5.88 (s, 1H), 4.75 (s, 2H), 4.09 (d, J=5.3 Hz, 2H), 3.95 (d, J=5.1 Hz, 2H), 2.12 (s, 3H), 1.76 (s, 3H), 1.67 (s, 3H).

### Example 12: Preparation of (2-amino-5-chloro-1-(3-hydroxy-2,6-dimethylphenyl)-1H-pyrrolo[2,3-b]pyridin-3-yl)(2-(trifluoromethyl)-5,6-dihydro-[1,2,4]triazolo[1,5-a]pyrazin-7(8H)-yl)methanone

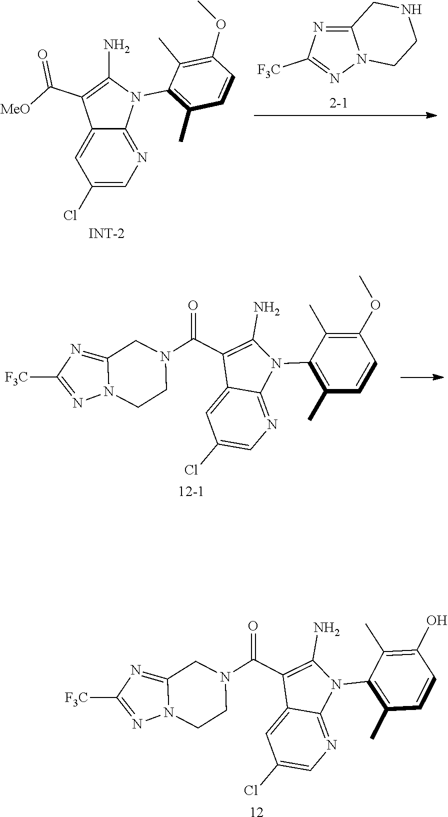

### Step 1: Preparation of Compound 12-1

To a solution of compound 2-1 (267 mg, 1.39 mmol) in dioxane (2 mL) was added DIPEA (179 mg, 1.39 mmol) and AlMe₃ (1.0 mL, 2.0 mmol, 2M in toluene). The mixture was stirred at 15 TC for 1 h. Compound INT-2 (0.39 mL, 0.78 mmol) was added. The mixture was stirred at 120° C. for 24 h. The reaction mixture was quenched by MeOH (1 mL), and filtered. The filtrate was concentrated to dryness and purified by Pre-TLC to afford compound 12-1 as a yellow solid. LCMS: 520.2 [M+H]+.

### Step 2: Preparation of Example 12

To a solution of compound 12-1 (10 mg, 0.019 mmol) in DCM (5 mL) was added BBr₃ (0.1 mL). The mixture was stirred at room temperature under N₂ for 1 h. The reaction mixture was quenched by MeOH (5 mL), and concentrated to dryness. The residue was purified by Pre-HPLC to afford example 12 as a white solid. LCMS: 506.2 [M+H]⁺. ¹H NMR: (400 MHz, DMSO-d₆) δ 9.55 (s, 1H), 7.79 (d, J=2.0 Hz, 1H), 7.64 (d, J=2.1 Hz, 1H), 7.07 (d, J=8.3 Hz, 1H), 6.92 (d, J=8.3 Hz, 1H), 6.65 (s, 2H), 4.91 (s, 2H), 4.36 (s, 2H), 4.06 (s, 2H), 1.76 (s, 3H), 1.68 (s, 3H).

### Example 13: Preparation of (6-amino-5-(3-hydroxy-2,6-dimethylphenyl)-2,3-dimethyl-5H-pyrrolo[2,3-b]pyrazin-7-yl)(3-chloro-6,7-dihydropyrazolo[1,5-a]pyrazin-5(4H)-yl)methanone

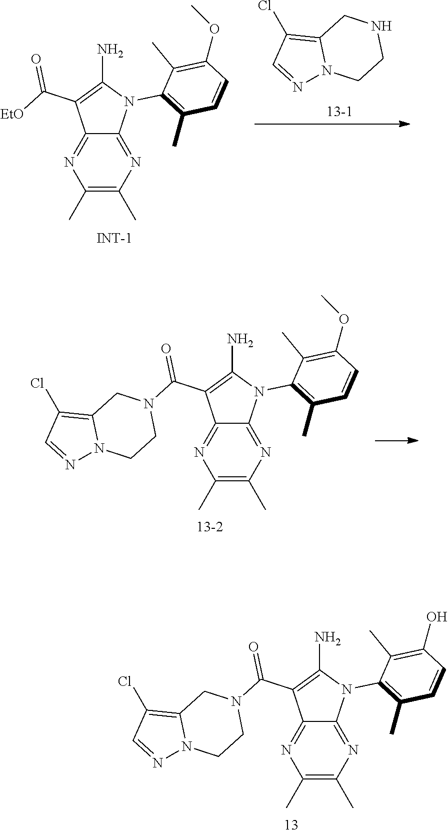

### Step 1: Preparation of Compound 13-2

To a solution of compound 13-1 (25.77 mg, 0.164 mmol) in dioxane (3 mL) was added DIEA (0.359 mL, 2.171 mmol) and AlMe₃ (0.078 mL, 2.171 mmol). The mixture was stirred at room temperature for 10 min. Then compound INT-1 (30 mg, 0.081 mmol) was added, the resulting reaction mixture was stirred at 120° C. under N₂ for 1 h. The reaction mixture was quenched by MeOH (1 mL), which was filtered. The filtrate was concentrated to dryness and purified by Pre-TLC to afford compound 13-2. LCMS: 480.1 [M+H]⁺.

### Step 2: Preparation of Example 13

To a solution of compound 13-2 (30 mg, 0.063 mmol) in DCM (5 mL) was added BBr₃ (0.1 mL). The mixture was stirred at room temperature under N₂ for 1 h. The reaction was quenched with MeOH (5 mL). The mixture was concentrated to dryness. The residue was purified by Pre-HPLC to afford example 13. LCMS: 466.1L[M+H]⁺; ¹H NMR: (400 MHz, DMSO-d₆) δ 9.56 (s, 1H), 7.61 (s, 1H), 7.12-7.05 (m, 3H), 6.93 (d, J=8.3 Hz, 1H), 4.83 (s, 2H), 4.41 (s, 2H), 4.12 (s, 2H), 2.46 (s, 3H), 2.31 (s, 3H), 1.78 (s, 3H), 1.69 (s, 3H).

### Example 14: Preparation of 5-(6-amino-5-(3-hydroxy-2,6-dimethylphenyl)-2,3-dimethyl-5H-pyrrolo[2,3-b]pyrazine-7-carbonyl)-4,5,6,7-tetrahydropyrazolo[1,5-a]pyrazine-3-carbonitrile

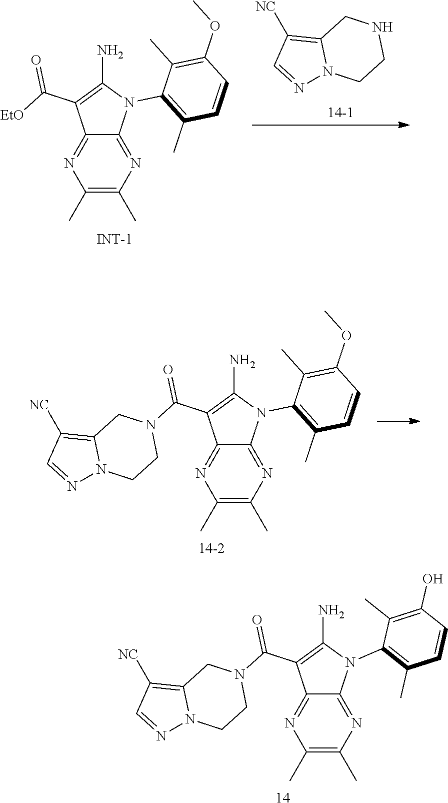

### Step 1: Preparation of Compound 14-2

To a solution of compound 14-1 (36.19 mg, 0.244 mmol) in dioxane (2 mL) was added DIEA (0.202 mL, 1.221 mmol) and trimethylaluminium (0.611 mL, 1.221 mmol). The mixture was stirred at 15° C. for 10 min. Then compound INT-1 (30 mg, 0.081 mmol) was added. The mixture was heated to 100° C. for 0.5 h. The reaction mixture was quenched with MeOH (1 mL), which was filtered. The filtrate was concentrated and purified by Pre-TLC to afford compound 14-2. LCMS: 471.1 [M+H]⁺.

### Step 2: Preparation of Example 14

To solution of compound 14-2 (30 mg, 0.064 mmol) in DCM (2 mL) was added BBr₃ (0.1 mL), and the reaction was stirred at rt for 0.5 h under N₂. The reaction was quenched with MeOH (3 mL) and NH₃/MeOH (3 mL). The mixture was concentrated. The residue was purified by Prep-TLC to afford example 14 as a yellow solid. LCMS: 457.1[M+H]⁺; ¹H NMR: (400 MHz, MeOD-d₄) δ 7.94 (s, 1H), 7.11 (d, J=8.3 Hz, 1H), 6.93 (d, J=8.3 Hz, 1H), 5.11 (s, 2H), 4.55 (s, 2H), 4.25 (t, J=5.4 Hz, 2H), 2.53 (s, 3H), 2.40 (s, 3H), 1.87 (s, 3H), 1.82 (s, 3H).

### Example 15: Preparation of (6-amino-5-(3-hydroxy-2,6-dimethylphenyl)-2,3-dimethyl-5H-pyrrolo[2,3-b]pyrazin-7-yl)(3-(trifluoromethyl)-6,7-dihydropyrazolo[1,5-a]pyrazin-5(4H)-yl)methanone

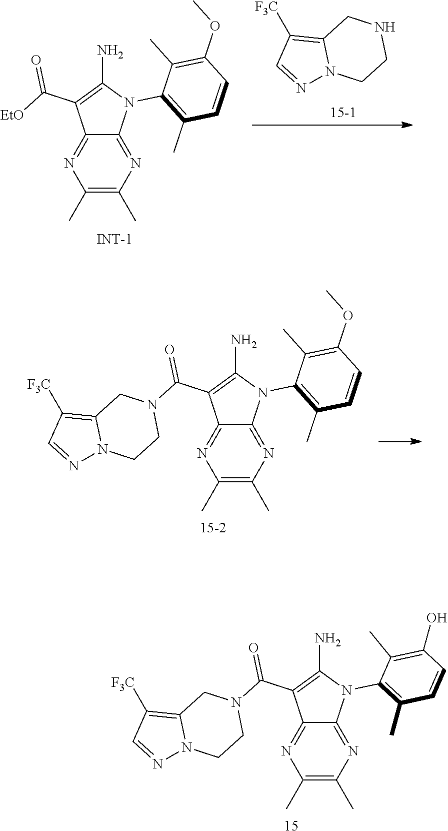

### Step 1: Preparation of Compound 15-2

To a solution of compound INT-1 (30 mg, 0.157 mmol) and compound 15-1 (115.64 mg, 0.314 mmol) in dioxane (2 mL) was added DIEA (0.259 mL, 1.569 mmol) and trimethylaluminium (113.14 mg, 1.569 mmol). The mixture was stirred at 100° C. for 1 h. The reaction mixture was carefully added into MeOH (10 mL) while stirring, which was adjusted to pH=7-8 with TEA. After filtration, the filtrate was concentrated to dryness. The residue was purified by Pre-TLC to afford compound 15-2. LCMS: 514.2 [M+H]⁺;

### Step 2: Preparation of Example 15

To a solution of compound 15-2 (50 mg, 0.097 mmol) in DCM (2 mL) was added BBr₃ (0.487 mL, 0.974 mmol). The mixture was stirred at rt for 1 h. The reaction mixture was carefully added into MeOH (10 mL) while stirring, which was adjusted to pH=7-8 with TEA. After filtration, the filtrate was concentrated to dryness. The residue was purified by Pre-HPLC to afford example 15. LCMS: 500.2 [M+H]⁺; ¹H NMR: (400 MHz, MeOD-d₄) δ 7.80 (s, 1H), 7.12 (d, J=8.3 Hz, 1H), 6.94 (d, J=8.3 Hz, 1H), 5.05 (s, 2H), 4.52 (t, J=5.0 Hz, 2H), 4.23 (t, J=5.4 Hz, 2H), 3.69 (s, 1H), 3.66 (s, 1H), 2.70 (s, 2H), 2.55 (s, 3H), 2.42 (s, 3H), 1.88 (s, 3H), 1.83 (s, 3H).

### Example 16: Preparation of (6-amino-5-(3-hydroxy-2,6-dimethylphenyl)-2,3-dimethyl-5H-pyrrolo[2,3-b]pyrazin-7-yl)(3-(hydroxymethyl)-6,7-dihydropyrazolo[1,5-a]pyrazin-5(4H)-yl)methanone

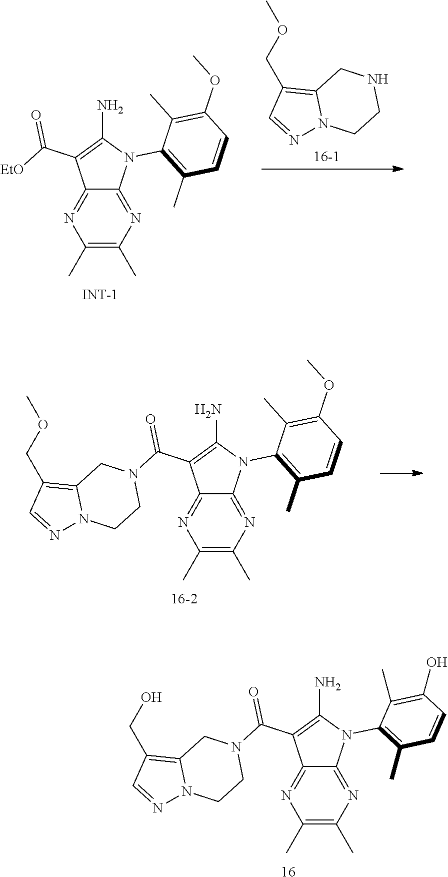

### Step 1: Preparation of Compound 16-2

To a solution of compound 16-1 (30 mg, 0.179 mmol) in dioxane (1 mL) was added DIEA (0.059 mL, 0.358 mmol) and AlMe₃ (25.83 mg, 0.358 mmol). The solution was stirred at rt for 10 min, and then compound INT-1 (13.2 mg, 0.036 mmol) was added to the solution. The mixture was stirred at 100° C. for 1 h under N₂. The mixture was quenched by MeOH and concentrated. The residue was purified by Pre-TLC to afford compound 16-2. LCMS: 490.2 [M+H]⁺.

### Step 2: Preparation of Example 16

To a solution of compound 16-2 (10 mg, 0.020 mmol) in DCE (1.5 mL) was added BBr₃ (102.34 mg, 0.409 mmol). The mixture was stirred at 80° C. for 16 h under N₂. The mixture was quenched by MeOH (3 mL) and concentrated. The residue was purified by Pre-HPLC to afford example 16. LCMS: 462.1 [M+H]⁺; ¹H NMR (400 MHz, MeOD-d₄) δ 7.50 (s, 1H), 7.11 (d, J=8.4 Hz, 1H), 6.94 (d, J=8.3 Hz, 1H), 4.98 (s, 2H), 4.50 (d, J=7.0 Hz, 4H), 4.22 (t, J=5.3 Hz, 2H), 2.54 (s, 3H), 2.40 (s, 3H), 1.87 (s, 3H), 1.82 (s, 3H).

### Example 17: Preparation of (6-amino-5-(3-hydroxy-2,6-dimethylphenyl)-2,3-dimethyl-5H-pyrrolo[2,3-b]pyrazin-7-yl)(2-methyl-6,7-dihydropyrazolo[1,5-a]pyrazin-5(4H)-yl)methanone

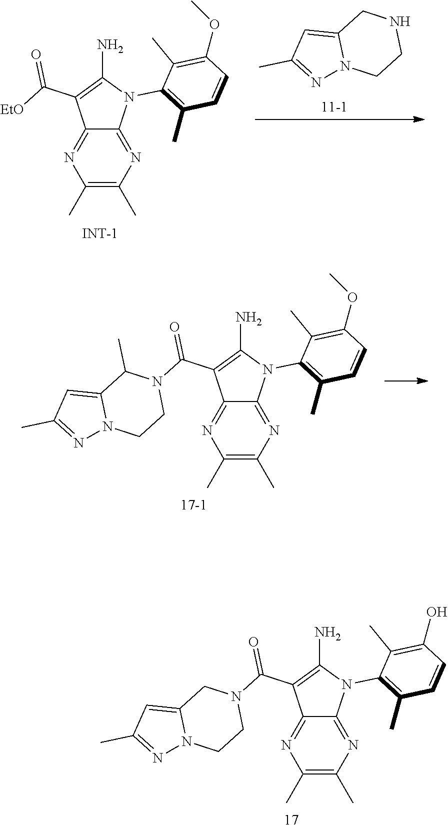

### Step 1: Preparation of Compound 17-1

To a solution of compound INT-1 (70 mg, 0.190 mmol) in dioxane (5 mL) were added compound 11-1 (33.9 mg, 0.247 mmol), and trimethylaluminium (0.950 mL, 1.900 mmol), and the reaction was stirred at 90° C. under N₂ for 2 h. The reaction mixture was diluted with water (30 mL) and MeOH (5 mL), which was extracted with EA (30 mL×3). Combined organic layers were washed with brine, dried over Na₂SO₄, filtered and concentrated. The residue was purified by silica gel column chromatography to afford compound 17-1. LCMS: 460.4 [M+H]⁺.

### Step 2: Preparation of Example 17

To a solution of compound 17-1 (60 mg, 0.131 mmol) in DCM (5 mL) was added BBr₃ (49.1 mg, 0.196 mmol), and the reaction was stirred at under N₂ for 3 h. The reaction mixture was diluted with water (20 mL), which was extracted with EA (30 mL×3). The combined organic layers were washed with brine (30 mL), dried over Na₂SO₄, filtered and concentrated. The residue was purified by Pre-HPLC to afford example 17. LCMS: 446.6[M+H]⁺; ¹H NMR (400 MHz, MeOD-d₄) δ 7.14 (d, J=8.4 Hz, 1H), 6.97 (d, J=8.3 Hz, 1H), 6.36 (s, 1H), 5.05 (s, 2H), 4.41 (s, 2H), 4.25-4.21 (m, 2H), 2.64 (s, 3H), 2.47 (s, 3H), 2.40 (s, 3H), 1.91 (s, 3H), 1.86 (s, 3H).

### Example 18: Preparation of (6-amino-5-(3-hydroxy-2,6-dimethylphenyl)-2,3-dimethyl-5H-pyrrolo[2,3-b]pyrazin-7-yl)(2-(trifluoromethyl)-6,7-dihydropyrazolo[1,5-a]pyrazin-5(4H)-yl)methanone

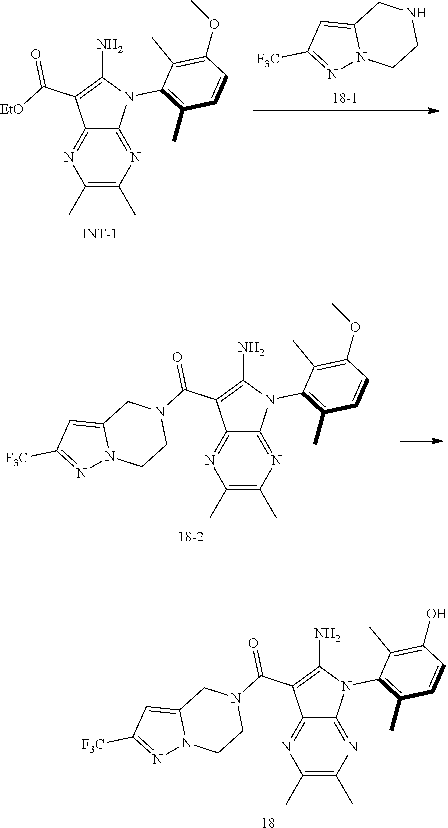

### Step 1: Preparation of Compound 18-2

To a solution of compound INT-1 (50 mg, 0.136 mmol) in dioxane (3 mL) was added compound 18-1 (46.51 mg, 0.204 mmol) and Me₃Al (0.34 mL, 2.0 M in hexane). The mixture was stirred at 90° C. under N₂ for 1 h. The reaction mixture was quenched by MeOH (10 mL), which was filtered. The filtrate was concentrated to afford compound 18-2. LCMS: 514.2[M+H]⁺.

### Step 2: Preparation of Example 18

To a solution of compound 18-2 (100 mg, 0.195 mmol) in DCM (5 mL) was added BBr₃ (0.487 mL, 2.0 M in DCM). The mixture was stirred at room temperature under N₂ for 0.5 h. The reaction was quenched with MeOH (5 mL). The mixture was concentrated to dryness. The residue was purified by Pre-HPLC to afford example 18. LCMS: 500.2 [M+H]⁺; ¹H NMR: (400 MHz, DMSO-d₆) δ 9.57 (s, 1H), 7.14 (s, 2H), 7.08 (d, J=8.3 Hz, 1H), 6.94 (d, J=8.3 Hz, 1H), 6.65 (s, 1H), 4.89 (s, 2H), 4.49 (s, 2H), 4.14 (s, 3H), 2.48 (s, 3H), 2.32 (s, 3H), 1.78 (s, 3H), 1.70 (s, 3H).

### Example 19: Preparation of (6-amino-5-(3-hydroxy-2,6-dimethylphenyl)-2,3-dimethyl-5H-pyrrolo[2,3-b]pyrazin-7-yl)(2-(trifluoromethyl)-6,7-dihydropyrazolo[1,5-a]pyrazin-5(4H)-yl)methanone

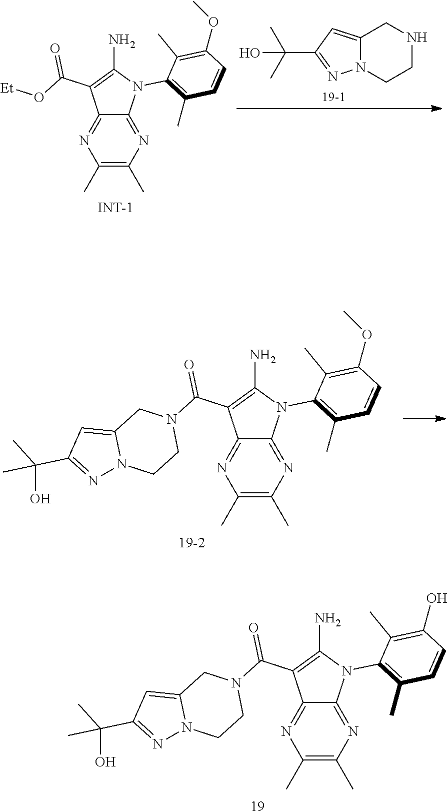

### Step 1: Preparation of Compound 19-2

To a solution of compound INT-1 (30 mg, 0.08 mmol) in dioxane (1 mL) were added compound 19-1 (118 mg, 0.65 mmol) and A1Me₃ (0.4 mL, 2 M, 0.8 mmol), the reaction was stirred at 120° C. for 18 h. MeOH (4 mL) was added to quench the reaction. The mixture was filtered. The filtrate was dried to afford crude compound 19-2. LCMS: 504.2 [M+H]+; Step 2: Preparation of Example 19

To a solution of compound 19-2 (20 mg) in DCM (4 mL) was added BBr₃ (0.1 mL), and the reaction was stirred at rt for 2 h. MeOH (2 mL) was added to quench the reaction, which was filtered and concentrate to dryness. The residue was purified by Pre-HPLC to afford example 19. LCMS: 490.3 [M+H]⁺. ¹H NMR: (400 MHz, DMSO-d₆) δ 9.62 (s, 1H), 8.43 (s, 1H), 7.38 (d, J=1.5 Hz, 1H), 7.10 (s, 2H), 7.07 (s, 1H), 6.93 (d, J=8.2 Hz, 1H), 6.41 (s, 1H), 4.32 (s, 2H), 4.13-3.97 (m, 2H), 3.18-3.08 (m, 2H), 2.47 (s, 3H), 2.31 (s, 3H), 1.80 (s, 3H) 1.78 (s, 3H), 1.68 (s, 3H), 1.54 (s, 3H).

### Example 20: Preparation of (6-amino-5-(3-hydroxy-2,6-dimethylphenyl)-2,3-dimethyl-5H-pyrrolo[2,3-b]pyrazin-7-yl)(3-methyl-6,7-dihydroisoxazolo[4,3-c]pyridin-5(4H)-yl)methanone

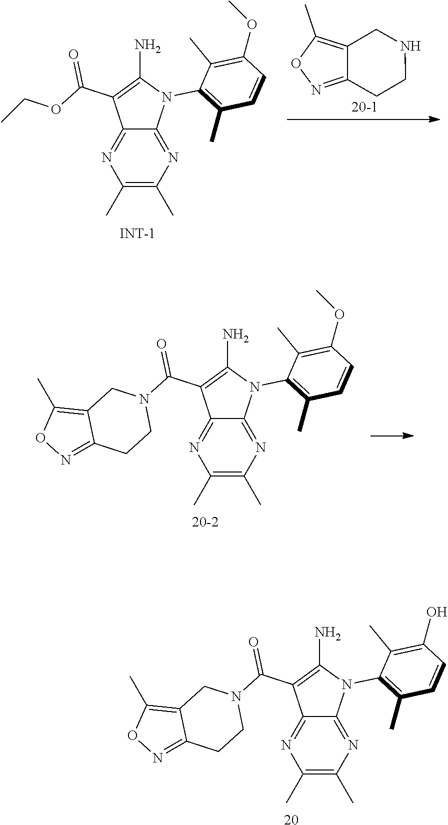

### Step 1: Preparation of Compound 20-2

To a solution of compound 20-1 (30.00 mg, 0.217 mmol) and compound INT-1 (40 mg, 0.11 mmol) in dioxane (1 mL) was added AlMe₃ (0.054 mL) dropwise at room temperature. The mixture was stirred at 90° C. for 1 h. The reaction mixture was carefully added into MeOH (10 mL) while stirring, which was neutralized to pH=7-8 with TEA. After filtration, the filtrate was concentrated to dryness. The residue was purified by column chromatography on silica gel to afford compound 20-2. LCMS: 461.5 [M+H]⁺.

### Step 2: Preparation of Example 20

To a solution of compound 20-2 (12 mg, 0.03 mmol) in DCM (3 mL) was added BBr₃ (32.64 mg, 0.130 mmol) stirred at rt. The mixture was stirred at rt for 1 h. MeOH (5 mL) was added. The mixture was concentrated. The residue was purified by Pre-TLC to afford example 20. LCMS: 447.5 [M+H]+; ¹H NMR: (400 MHz, DMSO-d₆) δ 9.60 (s, 1H), 7.18-7.09 (m, 3H), 6.94 (d, J=8.3 Hz, 1H), 4.56-4.46 (m, 2H), 3.89-3.84 (m, 2H), 3.07 (s, 2H), 2.48 (s, 3H), 2.35 (s, 3H), 2.32 (s, 3H), 1.78 (s, 3H), 1.70 (s, 3H).

### Example 21: Preparation of 2-(6-amino-5-(3-hydroxy-2,6-dimethylphenyl)-2,3-dimethyl-5H-pyrrolo[2,3-b]pyrazine-7-carbonyl)-1,2,3,4-tetrahydro-6H-pyrido[1,2-a]pyrazin-6-one

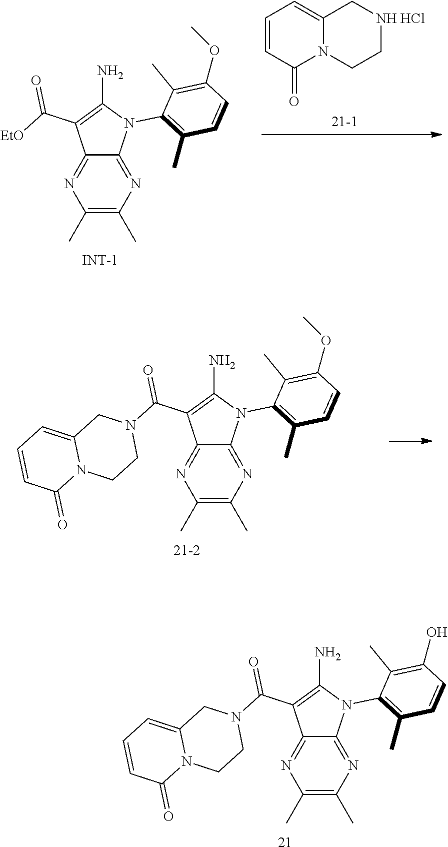

### Step 1: Preparation of Compound 21-2

To a solution of compound INT-1 (30 mg, 0.081 mmol) in the dioxane (2 mL) were added compound 21-1 (24 mg, 0.16 mmol) and trimethylaluminium (0.81 mL, 1M). The mixture was stirred at 100° C. for 3 h. MeOH (10 mL) was added to quench the reaction. The mixture was filtered and the filtrate was dried to afford compound 21-2. LCMS: 473.3 [M+H]⁺.

### Step 2: Preparation of Example 21

To a solution of compound 21-2 (30 mg) in DCM (3 mL) was added BBr₃ (0.1 mL), and the reaction was stirred at rt for 2 h. The mixture was quenched by MeOH (5 mL) and TEA (1 mL). The mixture was purified by Pre-HPLC to afford example 21. LCMS: 459.4 [M+H]⁺; ¹H NMR: (400 MHz, DMSO-d₆) δ 9.57 (s, 1H), 7.47-7.39 (m, 1H), 7.08 (d, J=8.3 Hz, 1H), 6.99 (s, 2H), 6.94 (d, J=8.2 Hz, 1H), 6.36 (d, J=9.7 Hz, 1H), 6.31 (s, 1H), 4.85 (s, 2H), 4.23 (s, 2H), 4.01 (s, 2H), 2.33 (s, 3H), 2.31 (s, 3H), 1.79 (s, 3H), 1.70 (s, 3H).

### Example 22: Preparation of (6-amino-5-(3-hydroxy-2,6-dimethylphenyl)-2,3-dimethyl-5H-pyrrolo[2,3-b]pyrazin-7-yl)(5,6-dihydroimidazo[1,2-a]pyrazin-7(8H)-yl)methanone

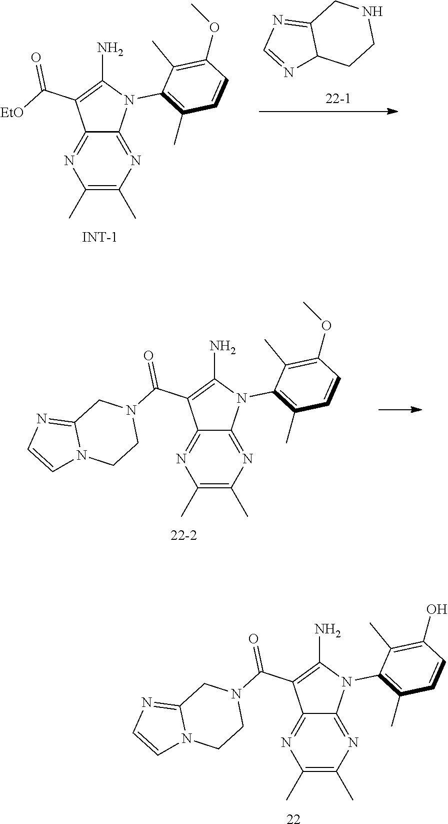

### Step 1: Preparation of Compound 22-2

To a solution of compound INT-1 (25 mg, 0.068 mmol) and compound 22-1 (41.87 mg, 0.340 mmol) in dioxane (1.5 mL) was added AlMe₃ (48.92 mg, 0.679 mmol). The mixture was stirred at 110° C. under N₂ for 1 h. The reaction mixture was carefully added into MeOH (10 mL) while stirring. After filtration, the filtrate was concentrated to dryness. The residue was purified by Pre-TLC to afford compound 22-2. LCMS: 446.3 [M+H]⁺.

### Step 2: Preparation of Example 22

To a solution of compound 22-2 (35 mg, 0.079 mmol) in DCM (6 mL) was added BBr₃ (0.1 mL). The mixture was stirred at rt for 1 h. The reaction mixture was carefully added into MeOH (10 mL) while stirring, the filtrate was concentrated to dryness. The residue was purified by Pre-HPLC to afford example 22. LCMS: 432.1 [M+H]⁺; ¹H NMR: (400 MHz, DMSO-d₆) δ 9.59 (s, 1H), 7.16 (s, 1H), 7.08 (d, J=8.2 Hz, 1H), 7.02 (s, 2H), 6.96-6.85 (m, 2H), 4.78 (s, 2H), 4.30 (s, 2H), 4.06 (s, 2H), 2.48 (s, 3H), 2.32 (s, 3H), 1.77 (s, 3H), 1.69 (s, 3H).

### Example 23: Preparation of (6-amino-5-(3-hydroxy-2,6-dimethylphenyl)-2,3-dimethyl-5H-pyrrolo[2,3-b]pyrazin-7-yl)(5,6-dihydroimidazo[1,5-a]pyrazin-7(8H)-yl)methanone

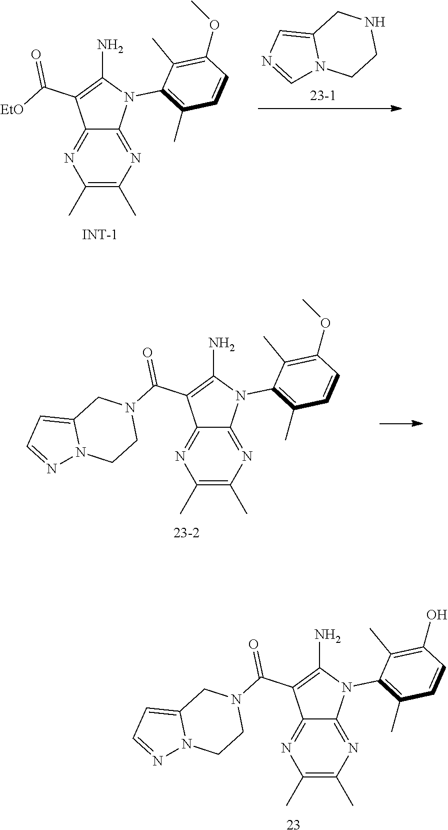

### Step 1: Preparation of Compound 23-2

To a solution of compound INT-1 (25 mg, 0.068 mmol) and compound 23-1 (41.78 mg, 0.339 mmol) in dioxane (1.5 mL) was added A1Me₃ (9.78 mg, 0.136 mmol). The mixture was stirred at 110° C. under N₂ for 1 h. The reaction mixture was carefully added into MeOH (10 mL) while stirring. After filtration, the filtrate was concentrated to dryness. The residue was purified by Pre-TLC to afford compound 23-2. LCMS: 446.3 [M+H]⁺.

### Step 2: Preparation of Example 23

To a solution of compound 23-2 (33 mg, 0.074 mmol) in DCM (6 mL) was added BBr₃ (0.1 mL). The mixture was stirred at rt for 1 h. The reaction mixture was carefully added into MeOH (10 mL) while stirring, the filtrate was concentrated to dryness. The residue was purified by Pre-HPLC to afford example 23. LCMS: 432.1 [M+H]⁺; ¹H NMR (400 MHz, DMSO-d₆) δ 9.59 (s, 1H), 7.65 (s, 1H), 7.08 (d, J=8.2 Hz, 1H), 7.01 (s, 2H), 6.94 (d, J=8.4 Hz, 1H), 6.77 (s, 1H), 4.82 (s, 2H), 4.36 (s, 2H), 4.01 (s, 2H), 2.48 (s, 3H), 2.32 (s, 3H), 1.78 (s, 3H), 1.69 (s, 3H).

### Example 24: Preparation of (6-amino-5-(3-hydroxy-2,6-dimethylphenyl)-2,3-dimethyl-5H-pyrrolo[2,3-b]pyrazin-7-yl)(3-(2-hydroxypropan-2-yl)-5,6-dihydroimidazo[1,5-a]pyrazin-7(8H)-yl)methanone

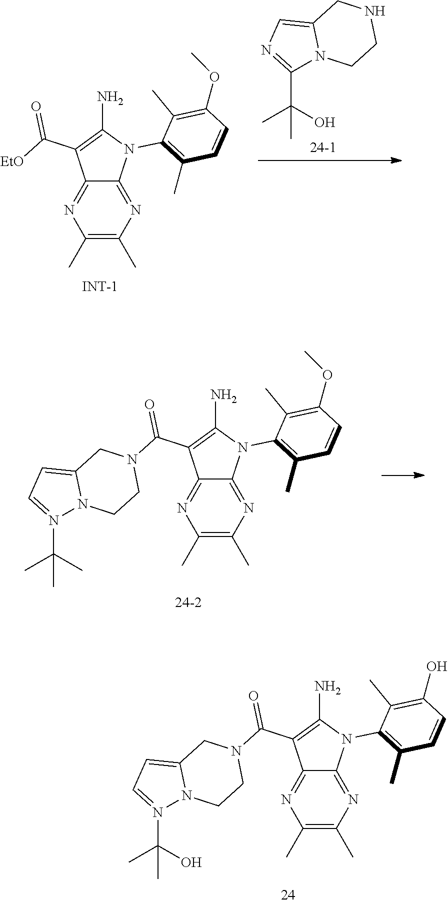

### Step 1: Preparation of Compound 24-2

To a solution of compound INT-1 (40 mg, 0.109 mmol) in dioxane (3 mL) was added compound 24-1 (39.35 mg, 0.217 mmol) and Me₃A (0.54 mL, 2.0 M in hexane). The mixture was stirred at 100° C. under N₂ for 24 h. The reaction mixture was quenched by MeOH (10 mL), which was filtered. The filtrate was concentrated to afford compound 24-2. LCMS: 504.3 [M+H]⁺.

### Step 2: Preparation of Example 24

To a solution of compound 24-2 (20 mg, 0.040 mmol) in DCM (5 mL) was added BBr₃ (0.2 mL). The mixture was stirred at room temperature under N₂ for 1 h. The reaction was quenched with MeOH (5 mL). The mixture was concentrated to dryness. The residue was purified by Pre-HPLC to afford example 24. LCMS: 490.3 [M+H]+; ¹H NMR: (400 MHz, MeOD-d₄) δ 7.38 (s, 1H), 7.14 (d, J=8.4 Hz, 1H), 6.97 (d, J=8.3 Hz, 1H), 5.03 (s, 2H), 4.89 (s, 2H), 4.22 (t, J=5.3 Hz, 2H), 2.57 (s, 3H), 2.43 (s, 3H), 1.89 (s, 3H), 1.84 (s, 3H), 1.77 (s, 6H).

### Example 25: Preparation of (6-amino-5-(3-hydroxy-2,6-dimethylphenyl)-2,3-dimethyl-5H-pyrrolo[2,3-b]pyrazin-7-yl)(7,8-dihydropyrido[3,4-b]pyrazin-6(5H)-yl)methanone

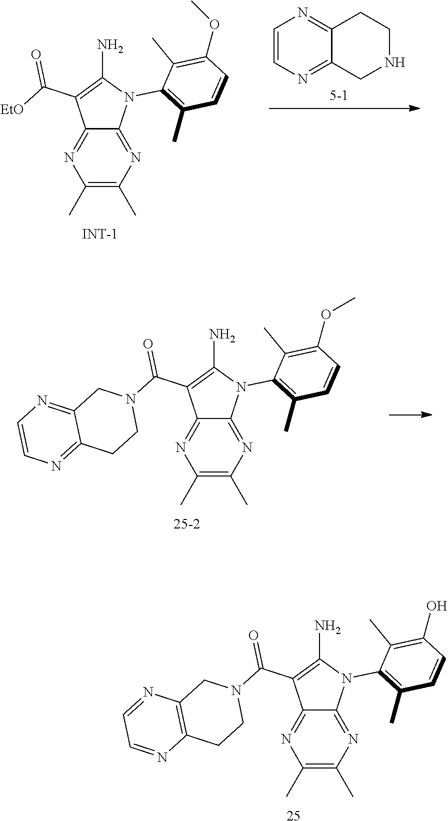

### Step 1: Preparation of Compound 25-2

To a solution of compound INT-1 (35 mg, 0.095 mmol) and compound 25-1 (64.20 mg, 0.475 mmol) in dioxane (1 mL) were added DIEA (122.78 mg, 0.950 mmol) and trimethylaluminium (0.475 mL). The reaction was stirred at 100° C. for 1 h. The mixture was concentrated and purified by silica gel column chromatography to afford compound 25-2. LCMS: 458.3 [M+H]⁺.

### Step 2: Preparation of Example 25

To a solution of compound 25-2 (35 mg, 0.076 mmol) in DCM (3 mL) was added BBr₃ (57.49 mg, 0.229 mmol). The reaction was stirred at rt for 1 h. The mixture was concentrated and purified by Pre-HPLC to afford example 25. LCMS: 444.2 [M+H]+; ¹H NMR (400 MHz, MeOD-d₄) δ 9.68 (s, 1H), 8.49 (s, 1H), 8.47 (s, 1H), 7.08-7.06 (m, 1H), 6.98-6.92 (m, 3H), 4.48 (s, 2H), 4.02 (t, J=5.4 Hz, 2H), 2.46 (s, 3H), 2.32 (s, 3H), 1.77 (s, 3H), 1.68 (s, 3H).

### Example 26: Preparation of (6-amino-5-(3-hydroxy-2,6-dimethylphenyl)-2,3-dimethyl-5H-pyrrolo[2,3-b]pyrazin-7-yl)(4-methyl-6,7-dihydropyrazolo[1,5-a]pyrazin-5(4H)-yl)methanone

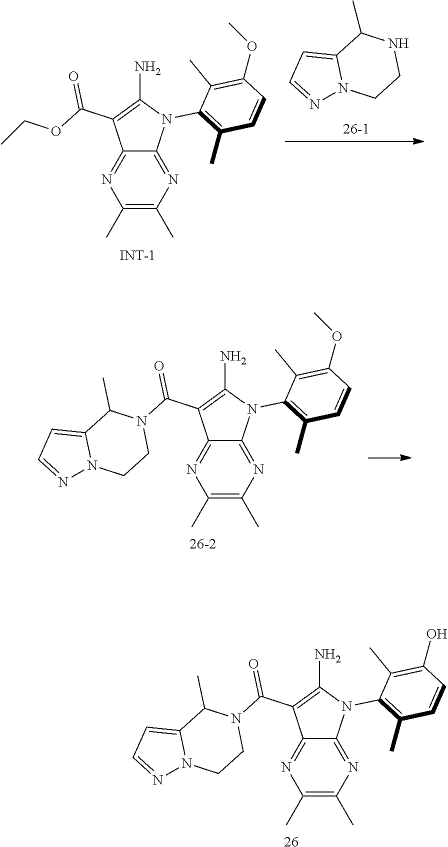

### Step 1: Preparation of Compound 26-2

To a solution of compound 26-1 (12 mg, 0.087 mmol) in dioxane (1 mL) was added compound INT-1 (16.11 mg, 0.044 mmol) and AlMe₃ (0.219 mL). The mixture was stirred at 90° C. for 1 h. The reaction mixture was carefully added into MeOH (10 mL) while stirring, which was adjusted to pH=7-8 with TEA. After filtration, the filtrate was concentrated to dryness. The residue was purified by column chromatography on silica gel to afford compound 26-2. LCMS: 460.1 [M+H]⁺.

### Step 2: Preparation of Example 26

To a solution of compound 26-2 (18.37 mg, 0.040 mmol) in DCM (2 mL) was added BBr₃ (29.99 mg, 0.120 mmol). The mixture was stirred at rt for 1 h. The reaction was quenched with MeOH (5 mL). The mixture was concentrated to dryness. The residue was purified by Pre-HPLC to afford example 26. LCMS: 446.2 [M+H]+; ¹H NMR: (400 MHz, DMSO-d₆) δ 9.57 (s, 1H), 7.43 (d, J=1.8 Hz, 1H), 7.06 (t, J=10.3 Hz, 3H), 6.94 (d, J=8.3 Hz, 1H), 6.16 (s, 1H), 5.69 (s, 1H), 4.59 (d, J=11.8 Hz, 2H), 4.28 (d, J=11.4 Hz, 1H), 3.65 (t, J=11.1 Hz, 1H), 2.47 (s, 3H), 2.31 (s, 3H), 1.77 (d, J=19.3 Hz, 3H), 1.69 (d, J=19.5 Hz, 3H), 1.57 (s, 3H), 1.55 (s, 3H).

### Example 27: Preparation of (2-amino-5-chloro-1-(3-hydroxy-2,6-dimethylphenyl)-1H-pyrrolo[2,3-b]pyridin-3-yl)(3-(trifluoromethyl)-5,6-dihydro-[1,2,4]triazolo[4,3-a]pyrazin-7(8H)-yl)methanone

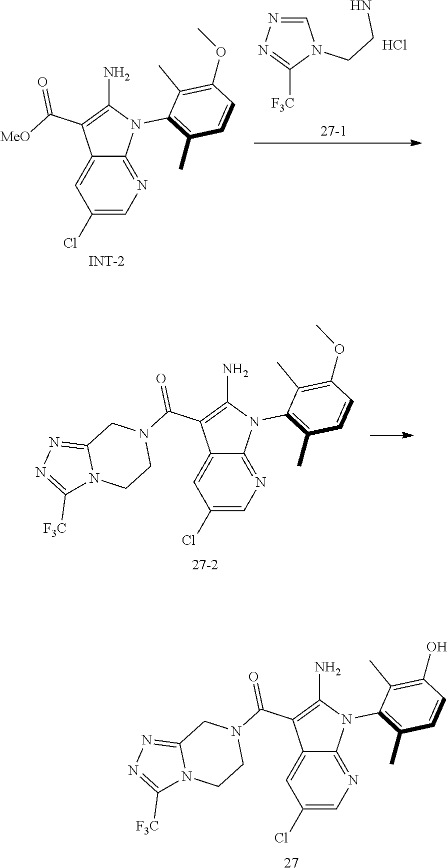

### Step 1: Preparation of Compound 27-2

To a solution of compound 27-1 (186.91 mg, 0.973 mmol) in dioxane (0.5 mL) was added trimethylaluminium (0.625 mL, 1.251 mmol). The mixture was stirred at 15° C. for 1 h. Then compound INT-2 (30 mg, 0.083 mmol) was added. The mixture was heated to 120° C. for 16 h. The reaction mixture was quenched by MeOH (10 mL), which was filtered. The filtrate was concentrated to dryness. The residue was purified by Pre-TLC to afford compound 27-2. LCMS: 520.4 [M+H]⁺.

### Step 2: Preparation of Example 27

To a solution of compound 27-2 (27 mg, 0.054 mmol) in DCM (2 mL) was added tribromoborane (0.1 mL, 0.005 mmol) at 0° C. then stirred at rt for 1 h. The mixture was quenched with MeOH (3 mL) and concentrated. The residue was purified by Pre-HPLC to afford example 27. LCMS: 506.3 [M+H]⁺; ¹H NMR: (400 MHz, DMSO-d₆) δ 9.53 (s, 1H), 7.79 (d, J=2.2 Hz, 1H), 7.65 (d, J=2.2 Hz, 1H), 7.07 (d, J=8.4 Hz, 1H), 6.92 (d, J=8.2 Hz, 11H), 6.64 (s, 2H), 4.96 (s, 2H), 4.26 (s, 2H), 3.98 (s, 2H), 1.76 (s, 3H), 1.68 (s, 3H).

### Example 28: Preparation of (6-amino-5-(2-chloro-3-hydroxy-6-methylphenyl)-2,3-dimethyl-5H-pyrrolo[2,3-b]pyrazin-7-yl)(6,7-dihydropyrazolo[1,5-a]pyrazin-5(4H)-yl)methanone

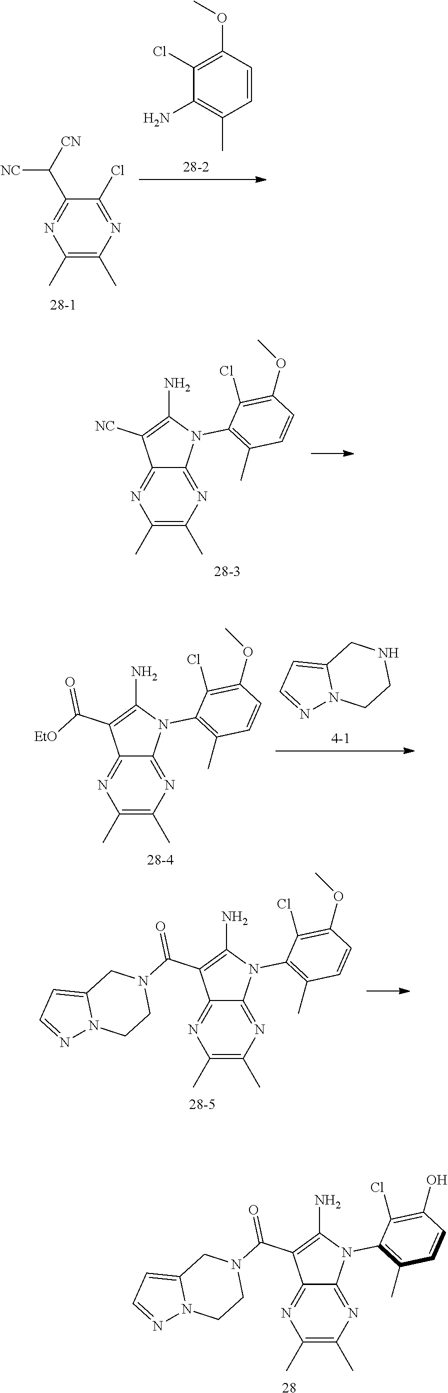

### Step 1: Preparation of Compound 28-3

To a solution of compound 28-1 (500 mg, 2.420 mmol) in NMP (10 mL) were added compound 28-2 (498 mg, 2.904 mmol), t-BuOK (815 mg, 7.259 mmol), and XantPhos-Pd-G₃ (6.92 mg, 0.007 mmol), and the reaction was stirred at 110° C. under N₂ for 0.5 h. The reaction mixture was diluted with water (20 mL), which was extracted with EA (30 mL×3). The combined organic layers were washed with brine (30 mL), dried over Na₂SO₄, filtered, and concentrated to dryness, concentrated in vacuo. The residue was purified by Pre-TLC to afford compound 28-3 (300 mg, 0.878 mmol, 36.27%). LCMS: 342.1 [M+H]⁺.

### Step 2: Preparation of Compound 28-4

To a solution of compound 28-3 (300 mg, 0.878 mmol) in EtOH (1.4 mL) and H₂SO₄ (0.2 mL) was stirred at room temperature overnight. The reaction mixture was carefully added into ice-water (10 mL) while stirring, which was adjusted to pH=8 with NaOH (4 M in water). The mixture was extracted with DCM (5 mL×3). The combined organic layer was washed with brine (10 mL), dried over Na₂SO₄, filtered, and concentrated to dryness. The organic layer was collected, concentrated in vacuo, and dried to afford compound 28-4. LCMS: 389.4 [M+H]⁺.

### Step 3: Preparation of Compound 28-5

To a solution of compound 4-1 (76 mg, 0.617 mmol) in dioxane (3 mL) were added DIEA (0.70 mL, 4.115 mmol) and AlMe₃ (0.30 mL, 4.115 mmol), the mixture was stirred at room temperature for 10 min. Then compound 28-4 was added, the mixture was stirred at 90° C. under N₂ for 2 h. The reaction mixture was quenched by MeOH (1 mL), which was filtered. The filtrate was concentrated to dryness and purified by Pre-TLC to afford compound 28-5. LCMS: 466.4 [M+H]⁺.

### Step 4: Preparation of Example 28

To a solution of compound 28-5 (110 mg, 0.0236 mmol) in DCM (5 mL) was added BBr₃ (0.1 mL). The mixture was stirred at rt under N₂ for 1 h. The reaction was quenched with MeOH (5 mL). The mixture was concentrated to dryness. The residue was purified by Pre-HPLC to afford example 28. LCMS: 452.4 [M+H]⁺; ¹H NMR: (400 MHz, DMSO-d₆) δ 7.45 (d, J=1.7 Hz, 1H), 7.14 (d, J=7.6 Hz, 3H), 7.04 (d, J=8.5 Hz, 1H), 6.15 (s, 1H), 4.85 (s, 2H), 4.41 (s, 2H), 4.18-4.03 (m, 2H), 2.47 (s, 3H), 2.32 (s, 3H), 1.83 (s, 3H).

### Example 29: Preparation of (6-amino-5-(4-fluoro-3-hydroxy-2,6-dimethylphenyl)-2,3-dimethyl-5H-pyrrolo[2,3-b]pyrazin-7-yl)(6,7-dihydropyrazolo[1,5-a]pyrazin-5(4H)-yl)methanone

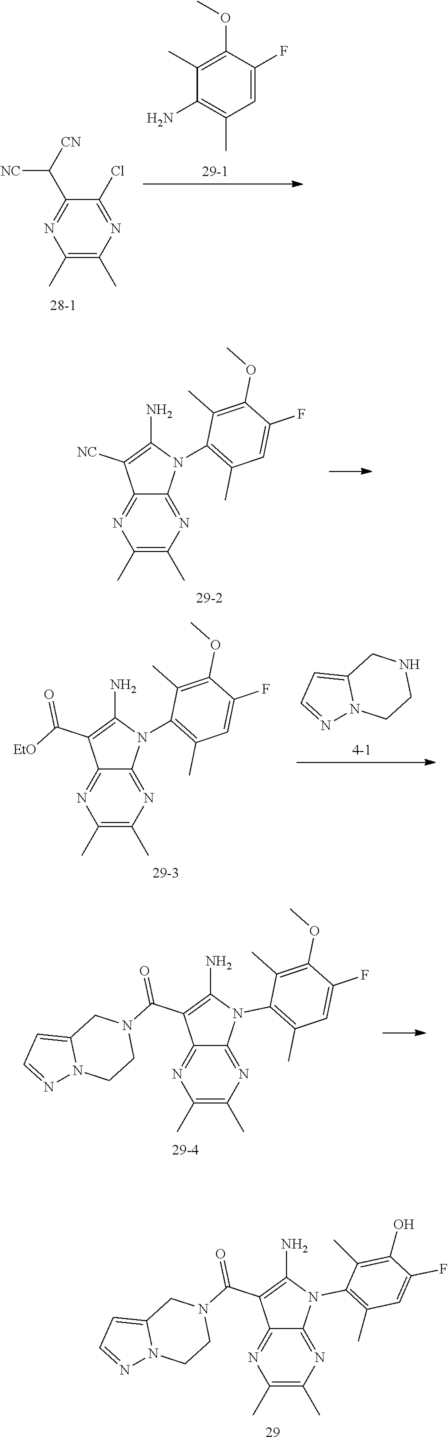

### Step 1: Preparation of Compound 29-2

To a solution of compound 28-1 (250 mg, 1.210 mmol) in NMP (5 mL) were added compound 29-1 (245.68 mg, 1.452 mmol), t-BuOK (406.56 mg, 3.630 mmol) and Pd-PEPPSITM-SIPr (41.26 mg, 0.060 mmol). The mixture was stirred at 110° C. under N₂ for 1.5 h. The reaction mixture was filtered. The filtrate was concentrated to dryness. The residue was purified by silica gel column chromatography to afford compound 29-2 (244 mg, 0.72 mmol, 59.43%). LCMS: 340.2 [M+H]⁺.

### Step 2: Preparation of Compound 29-3

A solution of compound 29-2 (447 mg, 1.317 mmol) in EtOH (10 mL) and H₂SO₄ (1.3 mL) was stirred at 90° C. under N₂ for 24 h. The mixture was poured into ice-water, which was neutralized to pH=7-8 with NaOH (4 M in water), which was extracted with EA (100 ml×3). The organic layer was combined and dried over Na₂SO₄. After filtration, the filtrate was concentrated and purified by silica gel column chromatography (100% EA) to afford compound 29-3. LCMS: 387.2 [M+H]⁺.

### Step 3: Preparation of Compound 29-4

To a solution of compound 29-3 (130 mg, 0.336 mmol) in dioxane (3 mL) were added compound 4-1 (49.72 mg, 0.404 mmol) and Me₃Al (1.112 mL). The mixture was stirred at 100° C. under N₂ for 1 h. The reaction mixture was quenched by MeOH (10 mL), which was filtered. The filtrate was concentrated to dryness and purified by Pre-TLC to afford compound 29-4. LCMS: 464.5 [M+H]⁺.

### Step 4: Preparation of Example 29

To a solution of compound 29-4 (100 mg, 0.216 mmol) in DCM (5 mL) was added BBr₃ (0.1 mL). The mixture was stirred at room temperature under N₂ for 1 h. The reaction was quenched with MeOH (5 mL). The mixture was concentrated to dryness. The residue was purified by Pre-HPLC to afford example 29. LCMS: 450.2 [M+H]⁺. ¹H NMR: (400 MHz, DMSO-d₆) δ 9.65 (s, 1H), 7.45 (s, 1H), 7.15-7.12 (m, 3H), 6.15 (s, 1H), 4.85 (s, 2H), 4.41 (s, 2H), 4.11 (s, 2H), 2.48 (s, 3H), 2.32 (s, 3H), 1.79 (s, 3H), 1.74 (s, 3H).

### Example 30: Preparation of racemic (6-amino-2-cyclopropyl-5-(3-hydroxy-2,6-dimethylphenyl)-3-methyl-5H-pyrrolo[2,3-b]pyrazin-7-yl)(6,7-dihydropyrazolo[1,5-a]pyrazin-5(4H)-yl)methanone

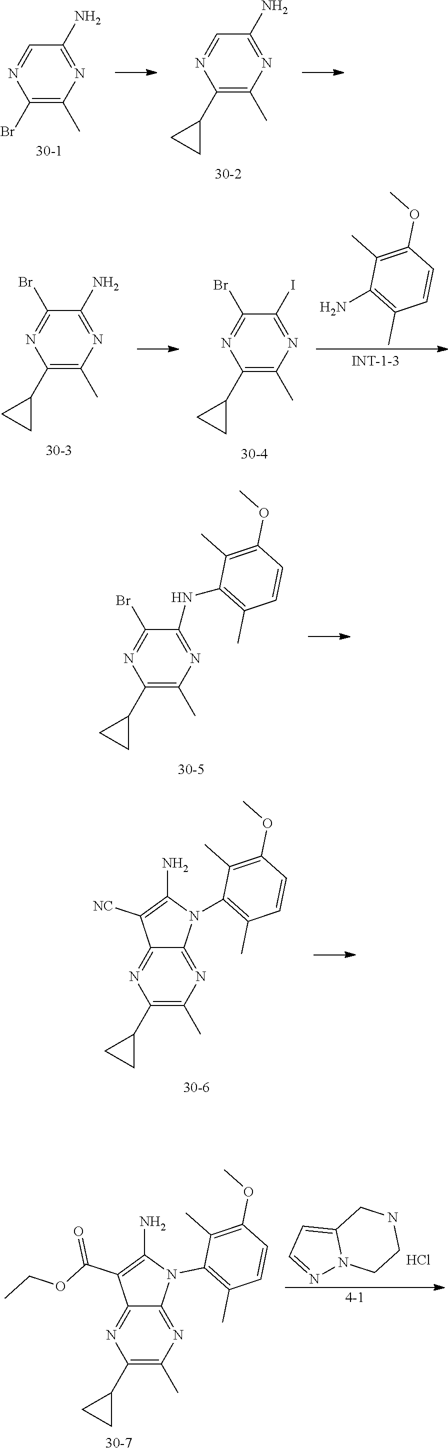

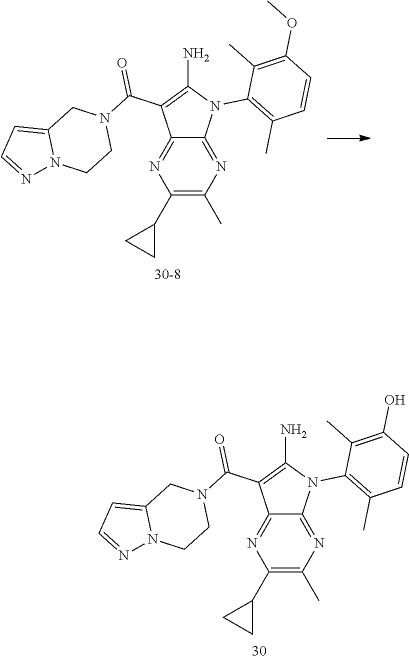

To a solution of compound 30-1 (5 g, 26.596 mmol) in dioxane-water (45 mL) were added cyclopropylborane (4.6 g, 53.191 mmol), Pd(dppf)Cl₂ (1.9 g, 2.660 mmol), and potassium carbonate (11.0 g, 79.787 mmol), and the reaction was stirred at 100° C. overnight. The reaction was diluted with EA (100 mL) and water (100 mL). The aqueous layer was extracted with EA (100 mL×3). Combined organic layers were washed with brine, dried over Na₂SO₄, filtered, and concentrated in vacuo. The residue was purified using silica gel column chromatography to afford compound 30-2 (3.716 g, 24.906 mmol, 93.6%) as brown solid. LCMS: 150.1 [M+H]⁺.

### Step 2: Preparation of Compound 30-3

To a solution of compound 30-2 (3.7 g, 24.906 mmol) in DMF (37 mL) was added NBS (8.4 g, 47.321 mmol), and the reaction was stirred at rt for 24 h. The reaction mixture was diluted with water (50 mL), which was extracted with EA (50 mL×3). The combined organic layer was washed with brine (100 mL×3), dried over Na₂SO₄, filtered, and concentrated to dryness. The residue was purified by silica gel column chromatography to afford compound 30-3 (1.985 g, 8.702 mmol, 34.9%) as a yellow solid. LCMS: 228.0 [M+H]⁺.

### Step 3: Preparation of Compound 30-4

To a solution of compound 30-3 (1.985 g, 8.702 mmol) in ACN (30 mL) were added isoamyl nitrite (1.5 g, 13.053 mmol), CuI (2.5 g, 13.053 mmol), and I₂ (3.3 g, 13.053 mmol) at 0° C., and the reaction was stirred at 60° C. for 3 h. The reaction was diluted with EA (50 mL) and saturated NaHSO₃ (50 mL). The organic layer was separated, washed with brine, dried over Na₂SO₄, filtered, and concentrated in vacuo. The residue was purified by silica gel column chromatography to afford compound 30-4 (881 mg, 2.599 mmol, 29.9%) as a yellow solid. LCMS: 338.9 [M+H]⁺.

### Step 4: Preparation of Compound 30-5

To a solution of compound 30-4 (881.0 mg, 2.599 mmol) in toluene (10 mL) were added compound INT-1-3 (392.9 mg, 2.599 mmol), Xantphos (300.7 mg, 0.520 mmol), Pd₂(dba)₃ (238.0 mg, 0.260 mmol) and Cs₂CO₃ (2540.1 mg, 7.796 mmol), and the reaction was stirred at 100° C. for 2 h. The mixture was concentrated and the residue was purified by silica gel column chromatography to afford compound 30-5 (545 mg, 1.504 mmol, 57.9%) as a as yellow solid. LCMS: 363.8 [M+H]⁺.

### Step 5: Preparation of Compound 30-6

To a solution of propanedinitrile (398.8 mg, 6.034 mmol) in DMF (3 mL) was added NaH (241.4 mg, 6.034 mmol) at 0 T under N₂. The mixture was stirred at 0° C. for 0.5 h. Then compound 30-5 (545 mg, 1.508 mmol) and XantPhos-Pd-G₃ (109.2 mg, 0.115 mmol) were added. The reaction mixture was stirred at 130 T for 1 h under N₂ by microwave. The mixture was quenched by H₂O (40 mL) and extracted with EA (50 mL×3). Combined organic layers were washed with brine, dried over Na₂SO₄, filtered and concentrated. The residue was purified by silica gel column chromatography to afford compound 30-6. LCMS: 348.2 [M+H]⁺.

### Step 6: Preparation of Compound 30-7

A solution of compound 30-6 (209 mg, 0.602 mmol) in EtOH (2.1 mL) and H₂SO₄ (0.3 mL) was stirred at 90° C. for 12 h. The reaction mixture was carefully added into ice-water (10 mL) while stirring, which was adjusted to pH=8 with NaOH (4 M in water). The mixture was extracted with DCM (10 mL×3). The combined organic layers were dried over Na₂SO₄, filtered, and concentrated to dryness. The residue was purified by Pre-TLC to afford compound 30-7. LCMS: 395.5 [M+H]⁺.

### Step 7: Preparation of Compound 30-8

To a solution of compound 4-1 (150.1 mg, 1.218 mmol) in dioxane (5 mL) were added DIEA (0.81 mL, 4.880 mmol) and trimethylaluminium (73.5 mg, 4.880 mmol). The mixture was stirred at rt for 10 min, and was added compound 30-7 (96 mg, 0.244 mmol). The mixture was stirred at 100° C. for 1 h. The mixture was quenched by MeOH and concentrated. The residue was purified by silica gel column chromatography to afford compound 30-8. LCMS: 472.3 [M+H]⁺.

### Step 8: Preparation of Example 30

To a solution of compound 30-8 (75 mg, 0.159 mmol) in DCM (7.5 mL) was added BBr₃ (398.4 mg, 1.590 mmol) at 0° C. The mixture was stirred at rt for 1 h. The residue was purified by prep-HPLC to afford example 30. LCMS: 458.5 [M+H]⁺. ¹H NMR: (400 MHz, DMSO-d₆) δ 9.57 (s, 1H), 7.44 (d, J=1.5 Hz, 1H), 7.12-7.00 (m, 3H), 6.93 (d, J=8.2 Hz, 1H), 6.13 (s, 1H), 4.90 (s, 2H), 4.28 (s, 2H), 4.13 (s, 2H), 2.31-2.07 (m, 1H), 1.77 (s, 3H), 1.68 (s, 3H), 0.98-0.84 (m, 4H).

### Example 31: Preparation of (6-amino-2-cyclopropyl-5-(3-hydroxy-2,6-dimethylphenyl)-3-methyl-5H-pyrrolo[2,3-b]pyrazin-7-yl)(6,7-dihydropyrazolo[1,5-a]pyrazin-5(4H)-yl)methanone

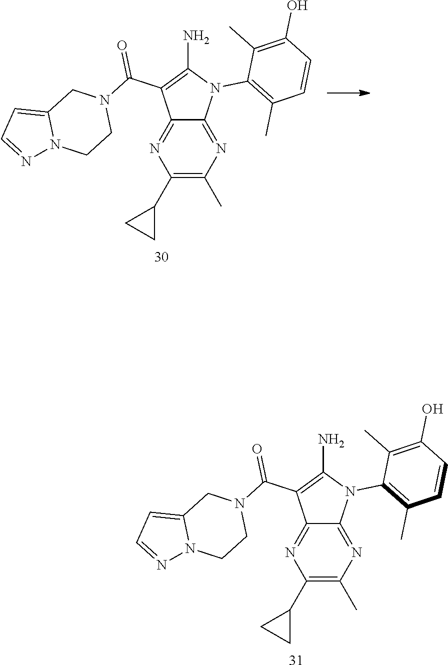

### Step 1: Preparation of Example 31

The example 30 was purified by prep-SFC to afford example 31. LCMS: 458.5 [M+H]⁺. ¹H NMR (400 MHz, DMSO-d₆) δ 9.57 (s, 1H), 7.44 (d, J=1.5 Hz, 1H), 7.12-7.00 (m, 3H), 6.93 (d, J=8.2 Hz, 1H), 6.13 (s, 1H), 4.90 (s, 2H), 4.28 (s, 2H), 4.13 (s, 2H), 2.31-2.07 (m, 1H), 1.77 (s, 3H), 1.68 (s, 3H), 0.98-0.84 (m, 4H).

### Example 32: Preparation of (6-amino-2-cyclopropyl-5-(3-hydroxy-2,6-dimethylphenyl)-5H-pyrrolo[2,3-b]pyrazin-7-yl)(6,7-dihydropyrazolo[1,5-a]pyrazin-5(4H)-yl)methanone

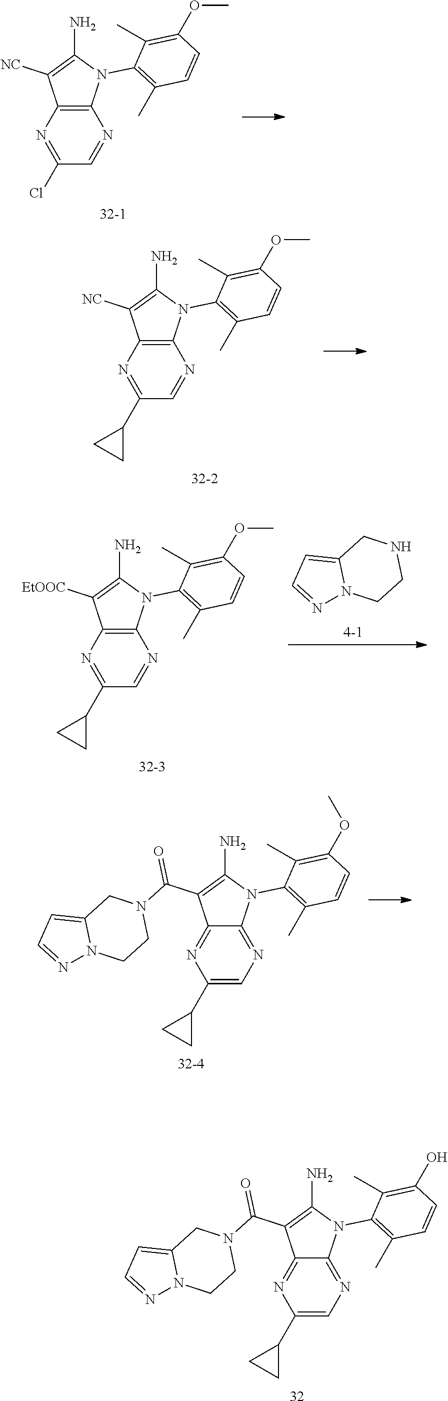

### Step 1: Preparation of Compound 32-2

To a solution of compound 32-1 (30 mg, 0.066 mmol) in DMF (2 mL) were added tributyl(cyclopropyl)-λ4-stannane (43.63 mg, 0.132 mmol), CuI (1.51 mg, 0.008 mmol), LiCl (5.58 mg, 0.132 mmol), Pd(dtbpf)Cl₂ (4.29 mg, 0.007 mmol). The mixture was stirred at 110° C. for 12 h. The reaction mixture was diluted with EA (10 mL), which was washed with brine (5 mL×3). The organic layer was dried over Na₂SO₄, filtered, and concentrated to dryness. The residue was purified by Pre-TLC to afford compound 32-2 (10 mg, 0.029 mmol, 43.69%) as yellow oil. LCMS: 334.1 [M+H]⁺.

### Step 2: Preparation of Compound 32-3

A solution of compound 32-2 (130 mg, 0.390 mmol) in EtOH (1.4 mL) and H₂SO₄ (0.2 mL) was stirred at 90° C. for 12 h. The reaction mixture was carefully added into ice-water (10 mL) while stirring, which was adjusted to pH=8 with NaOH (4 M in water). The mixture was extracted with DCM (10 mL×3). The combined organic layer was washed with brine (20 mL), dried over Na₂SO₄, filtered, and concentrated to dryness. The residue was purified by Pre-TLC to afford compound 32-3. LCMS: 381.0 [M+H]⁺.

### Step 3: Preparation of Compound 32-4

To a solution of compound 32-3 (28 mg, 0.074 mmol) and compound 4-1 (45.32 mg, 0.368 mmol) in dioxane (3 mL) were added trimethylaluminium (53.06 mg, 0.736 mmol) and DIEA (0.122 mL, 0.736 mmol). The mixture was stirred at 100° C. for 1 h. The reaction mixture was carefully added into MeOH (5 mL) while stirring, which was adjusted to pH=7-8 with TEA. After filtration, the filtrate was concentrated to dryness. The residue was purified by Pre-TLC to afford compound 32-4. LCMS: 458.2 [M+H]⁺;

### Step 4: Preparation of Example 32

To a solution of compound 32-4 (5 mg, 0.011 mmol) in DCM (2 mL) was added BBr₃ (0.05 mL, 0.109 mmol). The mixture was stirred at rt for 1 hr. The reaction mixture was carefully added into MeOH (5 mL) while stirring, which was adjusted to pH=7-8 with TEA. After filtration, the filtrate was concentrated to dryness. The residue was purified by Pre-HPLC to afford example 32. LCMS: 444.0 [M+H]⁺; ¹H NMR: (400 MHz, DMSO-d₆) δ 9.56 (s, 1H), 7.72 (s, 1H), 7.44 (s, 1H), 7.23 (s, 2H), 7.07 (d, J=8.5 Hz, 1H), 6.93 (d, J=8.2 Hz, 1H), 6.15 (s, 1H), 4.87 (s, 2H), 4.32 (s, 2H), 4.12 (s, 2H), 2.13 (s, 1H), 1.77 (s, 3H), 1.68 (s, 3H), 0.96-0.94 (m, 4H).

### Example 33: Preparation of (6-amino-5-(3-hydroxy-2,6-dimethylphenyl)-2-vinyl-5H-pyrrolo[2,3-b]pyrazin-7-yl)(6,7-dihydropyrazolo[1,5-a]pyrazin-5(4H)-yl)methanone

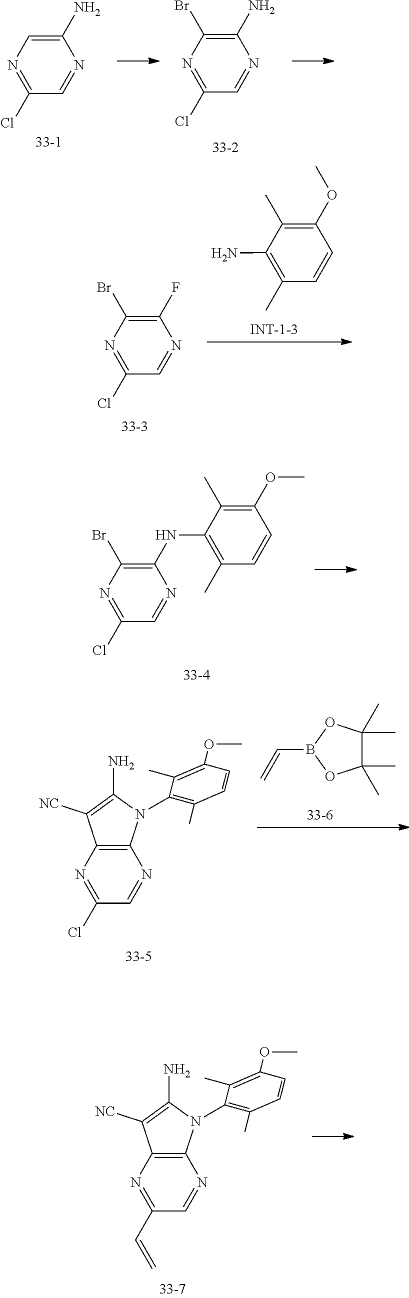

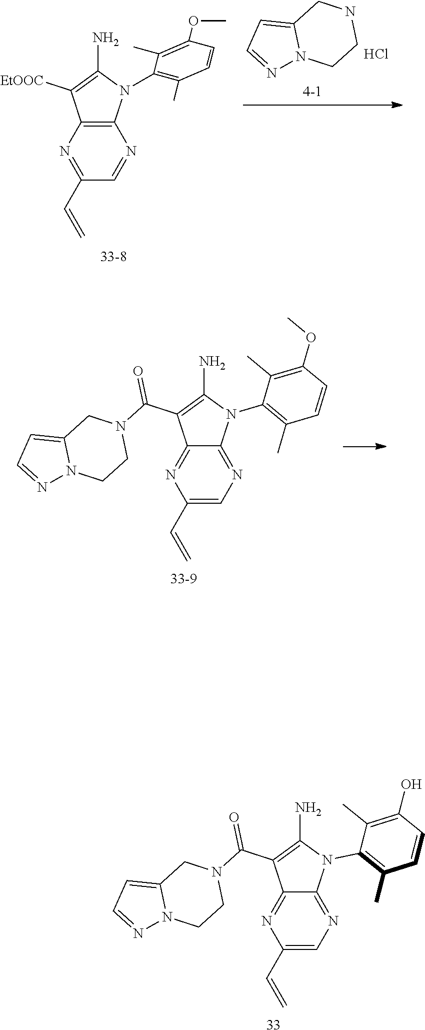

### Step 1: Preparation of Compound 33-2

To a solution of compound 33-1 (5 g, 38.595 mmol) in DCM (50 mL) was added NBS (6.87 g, 38.595 mmol) at 0° C. The mixture was stirred at room temperature for 1 h. The reaction mixture was diluted with DCM (250 mL), which was washed with brine (250 mL×3). The organic layer was dried over Na₂SO₄, filtered, and concentrated to dryness. The residue was purified by silica gel column chromatography to afford compound 33-2 (6.7 g, 83.28%) as a white solid. LCMS: 210.0 [M+H]⁺.

### Step 2: Preparation of Compound 33-3

To a solution of compound 33-2 (5 g, 23.988 mmol) in HF (55% in pyridine, 100 mL) was added NaNO₂ (3.31 g, 47.975 mmol) at 0° C. The mixture was stirred at rt under N₂ for 3 h. The reaction mixture was diluted with EA (150 mL), which was washed with sodium bicarbonate solution (150 mL×3). The organic layer was dried over Na₂SO₄, filtered, and concentrated to dryness. The residue was purified by silica gel column chromatography to afford compound 33-3 (300 mg, 5.92%) as colorless oil. ¹H NMR: (400 MHz, DMSO-d₆) δ 8.55 (d, J=1.6 Hz, 1H).

### Step 3: Preparation of Compound 33-4

To a solution of compound 33-3 (300 mg, 1.419 mmol) and compound INT-1-3 (214.56 mg, 1.419 mmol) in THF (9 mL) was added LiHMDS (712.31 mg, 4.257 mmol) at 0° C. under N₂. The mixture was stirred at room temperature under N₂ for 1 h. The reaction mixture was diluted with water (20 mL), which was extracted with EA (30 mL×3). The combined organic layer was washed with brine (90 mL), dried over Na₂SO₄, filtered, and concentrated to dryness. The residue was purified by silica gel column chromatography to afford compound 33-4 (500 mg, 102.84%) as a yellow solid. LCMS: 343.9 [M+H]⁺.

### Step 4: Preparation of Compound 33-5

To a solution of propanedinitrile (115.69 mg, 1.751 mmol) in DMF (10 mL) was added NaH (42.03 mg, 1.751 mmol) at 0° C. The mixture was stirred for 0.5 h. Then Pd(dppf)Cl₂—CH₂Cl₂ (119.18 mg, 0.146 mmol) and compound 33-4 (500 mg, 1.459 mmol) were added. The mixture was stirred at 130° C. under N₂ for 1 h. The reaction mixture was diluted with EA (50 mL), which was washed with brine (50 mL×3). The organic layer was dried over Na₂SO₄, filtered, and concentrated to dryness. The residue was purified by silica gel column chromatography to afford compound 33-5. LCMS: 328.0 [M+H]⁺.

### Step 5: Preparation of Compound 33-7

A mixture of compound 33-5 (100 mg, 0.305 mmol), compound 33-6 (234.95 mg, 1.525 mmol), Na₂CO₃ (80.84 mg, 0.763 mmol) and Pd(dtbpf)Cl₂ (19.95 mg, 0.031 mmol) in dioxane-water (2 mL-0.5 mL) was stirred at 120° C. under N₂ for 3 h. The combined organic layer was dried over Na₂SO₄, filtered, and concentrated to dryness. The residue was purified by Pre-TLC to afford compound 33-7 (91 mg, 93.39%) as a yellow solid. LCMS: 320.0 [M+H]⁺; Step 6: Preparation of Compound 33-8

To a solution of compound 33-7 (80 mg, 0.250 mmol) in EtOH (0.7 mL) was added H₂SO₄ (0.1 mL). The mixture was stirred at 90° C. overnight. The reaction mixture was carefully added into ice-water (10 mL) while stirring, which was neutralized to pH=8 with NaOH (4 M in water). The mixture was extracted with DCM (10 mL×3). The combined organic layer was washed with brine (30 mL), dried over Na₂SO₄, filtered, and concentrated to dryness. The residue was purified by Pre-TLC to afford compound 33-8. LCMS: 367.2 [M+H]⁺.

### Step 7: Preparation of Compound 33-9

A mixture of compound 4-1 (151.25 mg, 1.228 mmol), AIMe₃ (88.53 mg, 1.228 mmol) and DIEA (0.203 mL, 1.228 mmol) in dioxane (1 mL) was stirred for 15 min, then compound 33-8 (45 mg, 0.123 mmol) was added. The mixture was stirred at 110° C. under N₂ for 1 h. The reaction mixture was carefully added into MeOH (10 mL) while stirring. After filtration, the filtrate was concentrated to dryness. The residue was purified by Pre-TLC to afford compound 33-9. LCMS: 444.5 [M+H]⁺.

### Step 8: Preparation of Example 33

To a solution of compound 33-9 (5 mg, 0.011 mmol) in DCM (5 mL) was added BBr₃ (0.1 mL). The mixture was stirred at rt for 1 h. The reaction mixture was carefully added into MeOH (10 mL) while stirring, which was neutralized to pH=7-8 with NH₃/MeOH (4 M). After filtration, the filtrate was concentrated to dryness. The residue was purified by Pre-HPLC to afford example 33. LCMS: 430.1 [M+H]⁺; ¹H NMR: (400 MHz, MeOD-d₄) δ 7.78 (s, 1H), 7.50 (d, J=1.5 Hz, 1H), 7.12 (d, J=8.3 Hz, 1H), 6.94 (d, J=8.3 Hz, 1H), 6.86 (dd, J=17.3, 10.9 Hz, 1H), 6.19 (d, J=17.3 Hz, 2H), 5.38 (d, J=11.0 Hz, 1H), 5.04 (s, 2H), 4.56-4.48 (m, 3H), 4.27 (t, J=5.3 Hz, 2H), 1.89 (s, 3H), 1.84 (s, 3H).

### Example 34: Preparation of (6-amino-2-(3,6-dihydro-2H-pyran-4-yl)-5-(3-hydroxy-2,6-dimethylphenyl)-3-methyl-5H-pyrrolo[2,3-b]pyrazin-7-yl)(6,7-dihydropyrazolo[1,5-a]pyrazin-5(4H)-yl)methanone

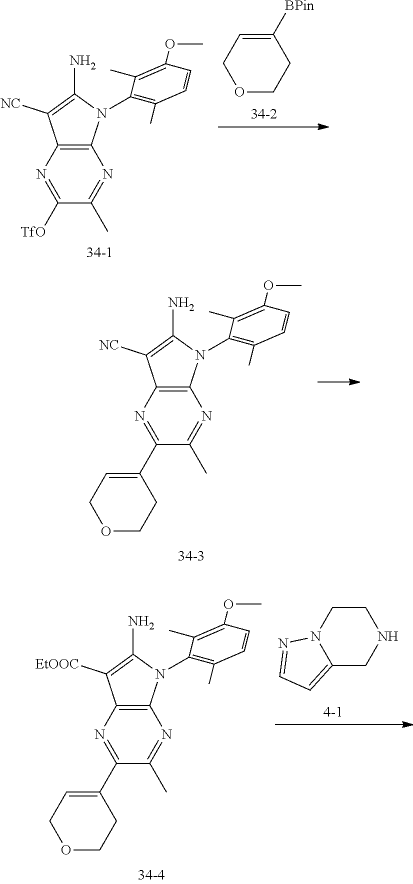

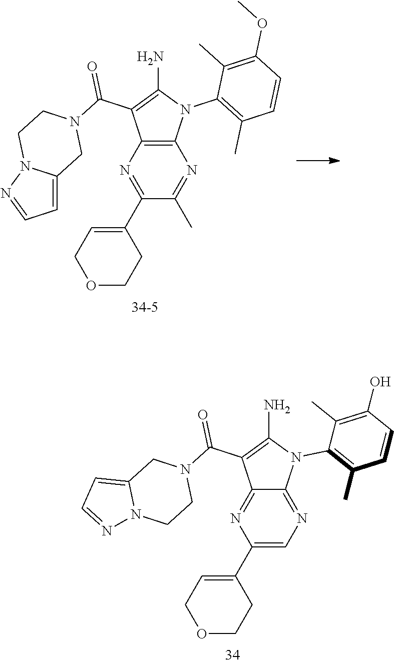

### Step 1: Preparation of Compound 34-3

To a solution of compound 34-1 (30 mg, 0.066 mmol) and compound 34-2 (41.52 mg, 0.198 mmol) in toluene (1 mL) and H₂O (0.1 mL) were added K₃PO₄ (41.95 mg, 0.198 mmol) and pd(dtbpf)Cl₂ (4.29 mg, 0.007 mmol). The mixture was stirred at 90° C. overnight. The reaction mixture was diluted with water (5 mL), which was extracted with EA (5 mL×3). The combined organic layer was washed with brine (30 mL), dried over Na₂SO₄, filtered, and concentrated to dryness. The residue was purified by Pre-TLC to afford compound 34-3 (5 mg, 19.49%) as a yellow solid. LCMS: 390.1 [M+H]⁺.

### Step 2: Preparation of Compound 34-4

A solution of compound 34-3 (5 mg, 0.013 mmol) in EtOH (1.4 mL) and H₂SO₄ (0.2 mL) was stirred at 90° C. for 12 h. The reaction mixture was carefully added into ice-water (2 mL) while stirring, which was adjusted to pH=8 with NaOH (4 M in water). The mixture was extracted with DCM (3 mL×3). The combined organic layer was washed with brine (10 mL), dried over Na₂SO₄, filtered, and concentrated to dryness. The residue was purified by Pre-TLC to afford compound 34-4. LCMS: 437.2 [M+H]⁺.

### Step 3: Preparation of Compound 34-5

To a solution of compound 4-1 (2.42 mg, 0.020 mmol) and compound 34-4 (4 mg, 0.010 mmol) in dioxane (1 mL) were added DIEA (0.004 mL, 0.025 mmol) and AlMe₃ (7.09 mg, 0.098 mmol), the mixture was stirred at 90° C. for 1 h. The reaction mixture was carefully added into MeOH (5 mL) while stirring, which was adjusted to pH=7-8 with TEA. After filtration, the filtrate was concentrated to dryness. The residue was purified by Pre-TLC to afford compound 34-5. LCMS: 514.2 [M+H]⁺;

### Step 4: Preparation of Example 34

A solution of compound 34-5 (3 mg, 0.006 mmol) in DCM (2 mL) was added BBr₃ (0.05 mL, 0.006 mmol), the mixture was stirred at 25° C. for 1 h. The reaction mixture was carefully added into MeOH (2 mL) while stirring, which was adjusted to pH=7-8 with TEA. After filtration, the filtrate was concentrated to dryness. The residue was purified by Pre-HPLC to afford example 34. LCMS: 500.2 [M+H]⁺; ¹H NMR: (400 MHz, DMSO-d₆) δ 7.44 (s, 1H), 7.22 (s, 2H), 7.09 (t, J=4.1 Hz, 2H), 6.95 (d, J=9.8 Hz, 2H), 6.13 (s, 1H), 5.95 (s, 1H), 4.92 (s, 2H), 4.30 (s, 2H), 4.25 (s, 2H), 4.14 (s, 2H), 3.86 (t, J=5.4 Hz, 2H), 2.41 (s, 3H), 2.34 (s, 2H), 1.80 (s, 3H), 1.72 (s, 3H).

### Example 35: Preparation of (6-amino-5-(3-hydroxy-2,6-dimethylphenyl)-2-methyl-3-(1-methylpiperidin-4-yl)-5H-pyrrolo[2,3-b]pyrazin-7-yl)(6,7-dihydropyrazolo[1,5-a]pyrazin-5(4H)-yl)methanone

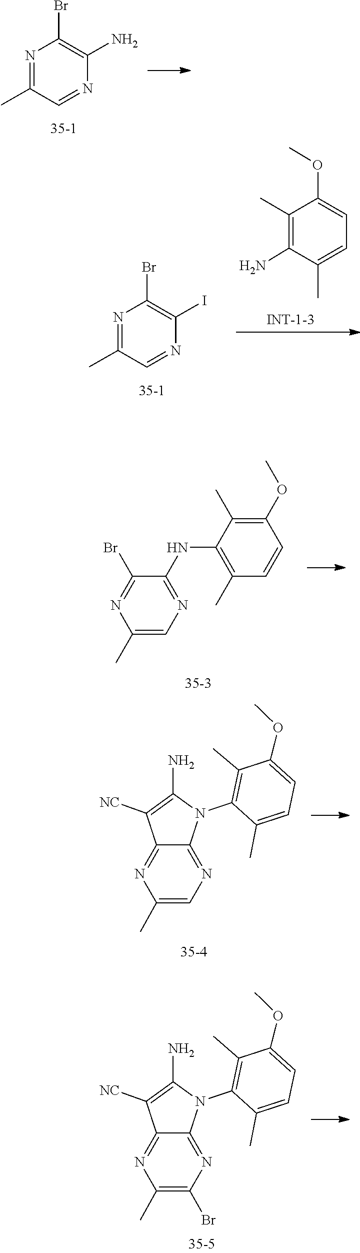

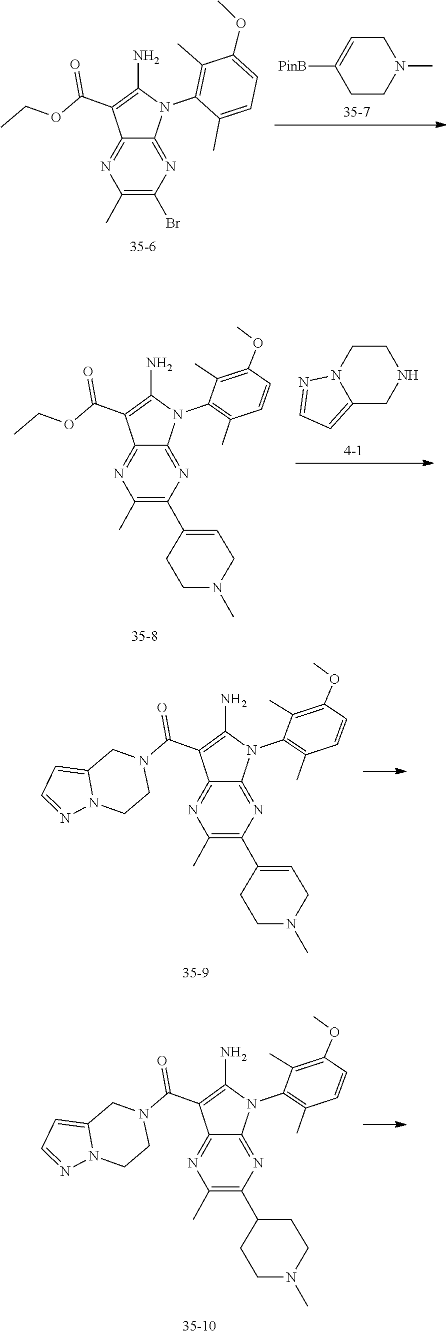

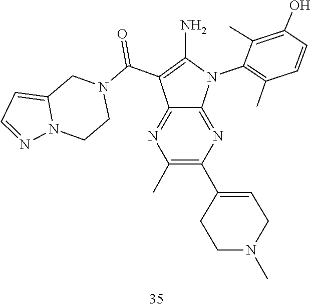 [SMILES: Cc1ccc(O)c(C)c1-n1c(N)c(C(=O)N2CCn3nccc3C2)c2nc(C)c(C3=CCN(C)CC3)nc21]

### Step 1: Preparation of Compound 35-2

To a solution of compound 35-1 (8.0 g, 42.546 mmol) in ACN (100 mL) was added CuI (10.51 g, 55.3 10 mmol), 12 (37.75 g, 85.093 mmol) and isoamyl nitrite (7.48 g, 63.820 mmol) at 0° C. The mixture was stirred at 70° C. for 2 h. The mixture was quenched with NH₄Cl (sat.aq, 50 mL), which was extracted with EA (500 mL). The organic layer was washed with Na₂SO₃ (sat.aq, 200 mL). The organic layer was dried over Na₂SO₄ and concentrated to dryness. The residue was purified by silica gel column chromatography to afford compound 35-2 (5.7 g, 44.82%) as a white solid.

### Step 2: Preparation of Compound 35-3

To a solution of compound 35-2 (5.7 g, 19.069 mmol) in dioxane (100 mL) were added compound INT-1-3 (3.17 g, 20.976 mmol), xantphos-Pd-G3 (1.81 g, 1.907 mmol) and Cs₂CO₃ (18.64 mg, 57.208 mmol). The mixture was stirred at 120° C. under N₂ for 2 h. The reaction mixture was filtered. The filtrate was concentrated to dryness. The residue was purified by silica gel column chromatography to afford compound 35-3 (4.4 g, 71.61%). LCMS: 323.9 [M+H]⁺.

### Step 3: Preparation of Compound 35-4

To a solution of propanedinitrile (1.80 g, 27.311 mmol) in DMF (60 mL) was added NaH (1.09 g, 27.311 mmol) at 0° C. The mixture was stirred at 0° C. for 0.5 h. Then compound 35-3 (4.4 g, 13.656 mmol) and xantphos-Pd-G3 (1.29 g, 1.366 mmol) were added. The mixture was stirred at 120° C. under N₂ for 2 h. The reaction mixture was filtered. The filtrate was concentrated to dryness. The residue was purified by silica gel column chromatography to afford compound 35-4 (1.1 g, 26.21%). LCMS: 308.1 [M+H]⁺.

### Step 4: Preparation of Compound 35-5

To a solution of compound 35-4 (180 mg, 0.586 mmol) in DMF (5 mL) was added NBS (125.08 mg, 0.703 mmol). The mixture was reacted at rt under N₂ for 0.5 h. The mixture was concentrated to dryness. The residue was purified by Pre-TLC to afford compound 35-5 (100 mg, 44.21%). LCMS: 386.1[M+H]⁺.

### Step 5: Preparation of Compound 35-6

A mixture of compound 35-5 (200 mg, 0.518 mmol) in H₂SO₄ (1 mL) and EtOH(7 mL) was stirred at under N₂ for 24 h. The mixture was poured into ice-water, which was neutralized to pH=8 with NaOH (4 M in water), which was extracted with EA (100 ml). The mixture was concentrated to dryness. The residue was purified by Pre-TLC to afford compound 35-6 (60 mg, 26.74%). LCMS: 389.2 [M+H-OEt]⁺.

### Step 6: Preparation of Compound 35-8

To a solution of compound 35-6 (40 mg, 0.092 mmol) in dioxane (3 mL) and H₂O (0.4 mL) were added compound 35-7 (25.94 mg, 0.184 mmol), K₂CO₃ (38.27 mg, 0.277 mmol) and Pd(dppf)Cl₂ (6.01 mg, 0.009 mmol). The mixture was stirred at 90° C. under N₂ for 1.5 h. The reaction mixture was filtered. The filtrate was concentrated to dryness. The residue was purified by silica gel column chromatography to afford compound 35-8. LCMS: 450.2 [M+H]⁺;

### Step 7: Preparation of Compound 35-9

To a solution of compound 35-8 (50 mg, 0.136 mmol) in dioxane (3 mL) were added compound 4-1 (20.10 mg, 0.163 mmol) and Me₃Al (0.33 mL, 2.0 M in toluene). The mixture was reacted at 120° C. under N₂ for 6 h. The reaction mixture was quenched by MeOH (10 mL), which was filtered. The residue was purified by Pre-TLC to afford compound 35-9. LCMS: 527.5 [M+H]⁺.

### Step 8: Preparation of Compound 35-10

To a solution of compound 35-9 (30 mg, 0.057 mmol) in MeOH (6 mL) was added Pd/C (20 mg, 10%). The mixture was reacted at 50° C. under H₂ for 5 h. The mixture was filtered, the filtrate was concentrated to dryness. The residue was purified by Pre-TLC to afford compound 35-10. LCMS: 529.4 [M+H]⁺.

### Step 9: Preparation of Example 35

To a solution of compound 35-10 (27 mg, 0.051 mmol) in DCM (5 mL) was added BBr₃ (0.1 mL). The mixture was stirred at room temperature under N₂ for 1 h. The reaction was quenched with MeOH (5 mL). The mixture was concentrated to dryness. The residue was purified by Pre-HPLC to afford example 35. LCMS: 515.4 [M+H]+; ¹H NMR: (400 MHz, MeOD-d₄) δ 7.54 (s, 1H), 7.07 (d, J=8.3 Hz, 1H), 6.89 (d, J=8.3 Hz, 1H), 6.21 (s, 1H), 4.96 (s, 2H), 4.50 (s, 2H), 4.22 (s, 2H), 3.50 (d, J=12.0 Hz, 2H), 3.25 (s, 1H), 3.11 (t, J=11.5 Hz, 2H), 2.81 (s, 3H), 2.63 (s, 3H), 2.08-1.91 (m, 4H), 1.86 (s, 3H), 1.81 (s, 3H).

### Example 36: Preparation of (6-amino-5-(3-hydroxy-2,6-dimethylphenyl)-3-(trifluoromethyl)-5H-pyrrolo[2,3-b]pyrazin-7-yl)(6,7-dihydropyrazolo[1,5-a]pyrazin-5(4H)-yl)methanone

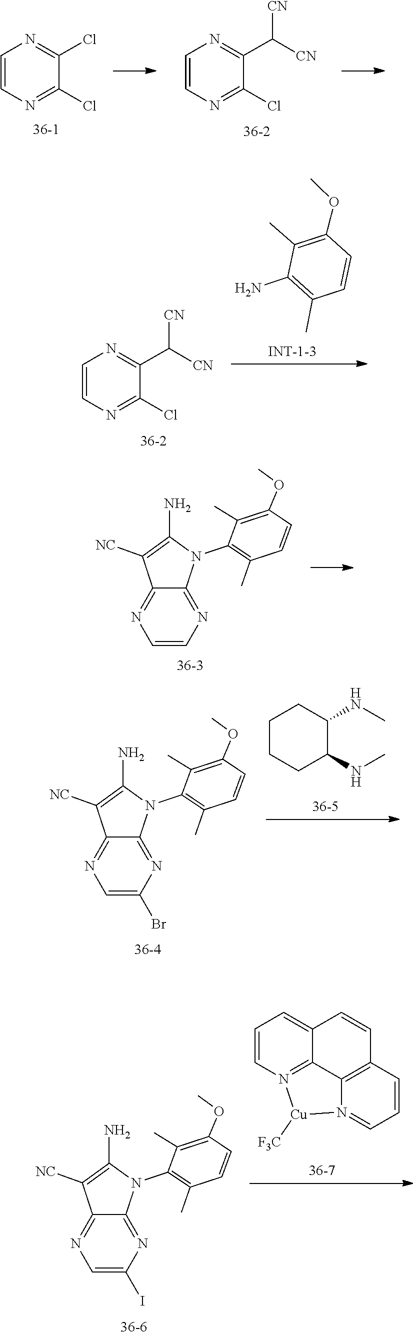

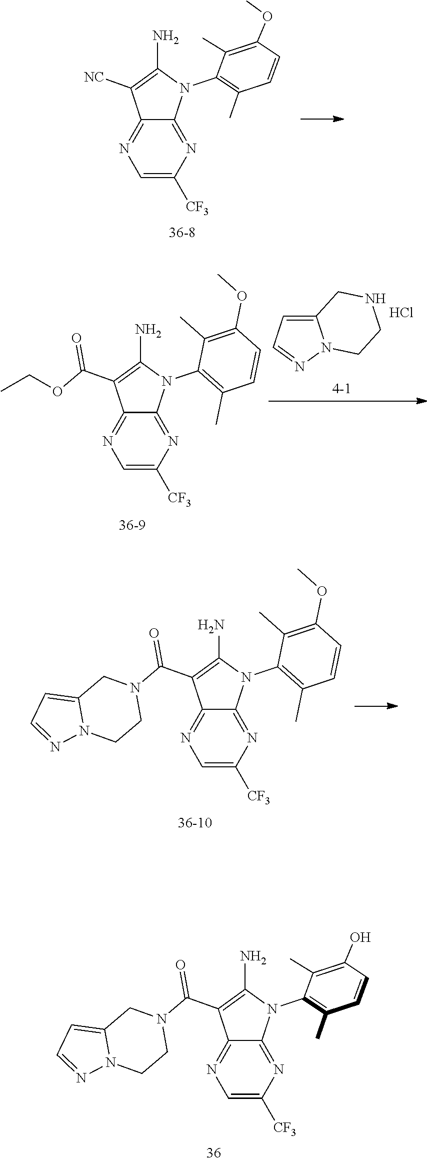

### Step 1: Preparation of Compound 36-2

To a solution of propanedinitrile (1.77 g, 26.851 mmol) in DME (50 mL) was added NaH (0.71 g, 29.536 mmol) in portions at 0° C. The mixture was stirred at 0° C. for 1 h. Then compound 36-1 (2 g, 13.426 mmol) was added. The mixture was heated to 90° C. for 1 h. The mixture was quenched by H₂O (20 mL) and adjust to PH=1 by 1N HCl and the suspension was stirred at rt for 10 min. The suspension was filtered and the solid was washed with H₂O and EtOH, dried to afford compound 36-2 (2 g, 11.199 mmol, 83.42%) as a yellow solid. ¹H NMR: (400 MHz, DMSO-d₆) δ 7.89 (d, J=2.7 Hz, 1H), 7.53 (s, 1H).

### Step 2: Preparation of Compound 36-3

A mixture of compound 36-2 (4 g, 22.399 mmol), compound INT-1-3 (6.8 g, 44.798 mmol), t-BuOK (7.5 g, 67.197 mmol) and Pd-PEPPSITM-SIPr (1.5 g, 2.240 mmol) in dioxane-water (5:1) (2 mL) was heated to 90° C. for 3 h. The mixture was concentrated and the residue was purified by column chromatography on silica gel to afford compound 36-3 (2 g, 6.818 mmol, 30.44%). LCMS: 294.1 [M+H]⁺.

### Step 3: Preparation of Compound 36-4

A mixture of compound 36-3 (1 g, 3.409 mmol) and NBS (0.6 g, 3.409 mmol) in DMF (20 mL) was stirred at rt for 20 min. The mixture was diluted with H₂O (10 mL), extracted with EA (10 mL×3). Combined organic layers were washed with brine, dries over Na₂SO₄, filtered and concentrated. The residue was purified by Pre-TLC to afford compound 36-4 (800 mg, 63.04%). LCMS: 415.1[M+MeCN+H]⁺.

### Step 4: Preparation of Compound 36-6

A mixture of compound 36-4 (1.2 g, 3.224 mmol), NaI (1.5 g, 9.671 mmol) and compound 36-5 (0.09 g, 0.645 mmol) and CuI (0.12 g, 0.645 mmol) in dioxane (20 mL) was heated to 110 T for 16 h under N₂. The mixture was concentrated and the residue was purified by column chromatography on silica gel to afford compound 36-6 (600 mg, 44.39%). LCMS: 461.0 [M+MeCN+H]⁺.

### Step 5: Preparation of Compound 36-8

A mixture of compound 36-6 (150 mg, 0.358 mmol) and compound 36-7 (223.8 mg, 0.716 mmol) in DMF (0.5 mL) was heated to 100 T for 8 h. The mixture was heated to 100 T for 1 h under N₂ by microwave. The mixture was diluted with H₂O (10 mL) and extracted with EA (10 mL×3). Combined organic layers were washed with brine, dried over Na₂SO₄, filtered and concentrated. The residue was purified by column chromatography on silica gel to compound 36-8 (30 mg, 23.20%). LCMS: 362.1[M+H]⁺.

### Step 6: Preparation of Compound 36-9

A solution of compound 36-8 (30 mg, 0.083 mmol) in EtOH/H₂SO₄ (10:1) (1 mL) was heated to 90° C. for 16 h. The mixture was concentrated and the residue was diluted with sat.aq NaHCO₃ (10 mL), extracted with EA (10 mL×3). Combined organic layers were washed with brine, dried over Na₂SO₄, filtered and concentrated. The residue was purified by Pre-TLC to afford compound 36-9. LCMS: 409.1 [M+H]⁺.

### Step 7: Preparation of Compound 36-10

To a solution of compound 4-1 (23.5 mg, 0.147 mmol) and DIEA (0.081 mL, 0.490 mmol) in dioxane (0.5 mL) was added AlMe₃ (0.25 mL) in drops at rt. The mixture was stirred at rt for 10 min and then compound 36-9 (20 mg, 0.049 mmol) was added. The reaction mixture was heated to 110° C. for 1 h. The mixture was quenched by MeOH (10 mL) and concentrated. The residue was diluted with MeOH/DCM (1:10, 10 mL) and filtered. The filtrated was concentrated and the residue was purified by Pre-TLC to afford compound 36-10. LCMS: 486.1[M+H]⁺.

### Step 8: Preparation of Example 36

To a solution of compound 36-10 (10 mg, 0.021 mmol) in DCM (1 mL) was added BBr₃ (0.21 mL, 0.206 mmol). The mixture was stirred at rt for 1 h. The mixture was quenched by MeOH (2 mL) and concentrated. The residue was purified by Pre-HPLC to afford example 36. LCMS: 472.2 [M+H]⁺; ¹H NMR: (400 MHz, MeOD-d₄) δ 8.56 (s, 1H), 7.54 (d, J=1.7 Hz, 1H), 7.15 (d, J=8.3 Hz, 1H), 6.99 (d, J=8.3 Hz, 1H), 6.21 (s, 1H), 5.01 (s, 2H), 4.50-4.46 (m, 2H), 4.24 (t, J=5.3 Hz, 2H), 1.91 (s, 3H), 1.85 (s, 3H).

### Example 37: Preparation of (R)-(6-amino-5-(3-hydroxy-2,6-dimethylphenyl)-3-(3-hydroxypiperidin-1-yl)-2-methyl-5H-pyrrolo[2,3-b]pyrazin-7-yl)(6,7-dihydropyrazolo[1,5-a]pyrazin-5(4H)-yl)methanone

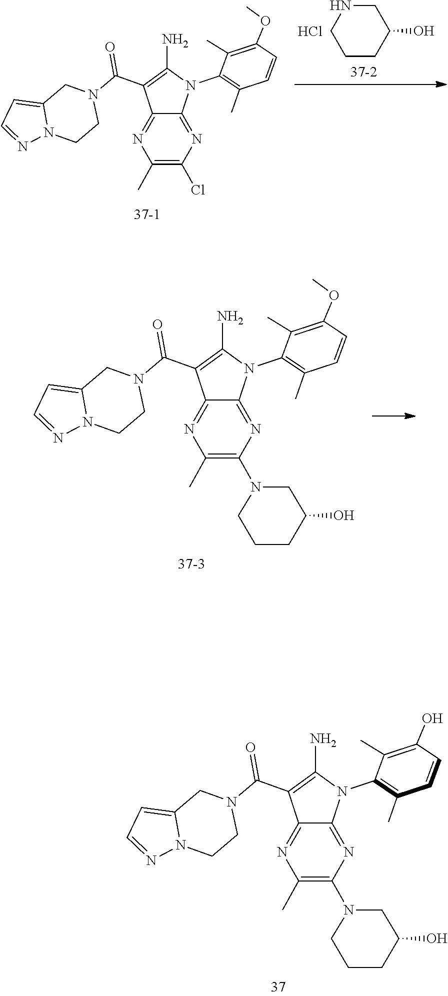

### Step 1: Preparation of Compound 37-3

To a solution of compound 37-1 (85 mg, 0.182 mmol) in dioxane (5 mL) were added compound 37-2 (55.36 mg, 0.547 mmol), xantphos-Pd-G3 (17.29 mg, 0.018 mmol) and Cs₂CO₃ (178.32 mg, 0.547 mmol). The mixture was stirred at 120° C. under N₂ for 24 h. The reaction mixture was filtered. The filtrate was concentrated to dryness. The residue was purified by Pre-TLC to afford compound 37-3. LCMS: 531.0 [M+H]⁺;

### Step 2: Preparation of Example 37

To a solution of compound 37-3 (30 mg, 0.057 mmol) in DCM (5 mL) was added BBr₃ (0.1 mL). The mixture was stirred at rt under N₂ for 1 h. The reaction was quenched with MeOH (5 mL). The mixture was concentrated to dryness. The residue was purified by Pre-HPLC to afford example 37. LCMS: 517.1 [M+H]⁺; ¹H NMR: (400 MHz, MeOD-d₄) δ 7.51 (s, 1H), 7.10 (d, J=8.2 Hz, 1H), 6.93 (d, J=8.2 Hz, 1H), 6.18 (s, 1H), 5.01 (s, 2H), 4.45 (s, 2H), 4.21 (s, 1H), 3.81 (s, 1H), 2.74-2.59 (m, 4H), 1.89 (s, 6H), 1.84 (s, 3H) 1.33-1.29 (m, 4H).

### Example 38: Preparation of (6-amino-5-(3-hydroxy-2,6-dimethylphenyl)-2-(trifluoromethyl)-5H-pyrrolo[3,2-d]pyrimidin-7-yl)(6,7-dihydropyrazolo[1,5-a]pyrazin-5(4H)-yl)methanone

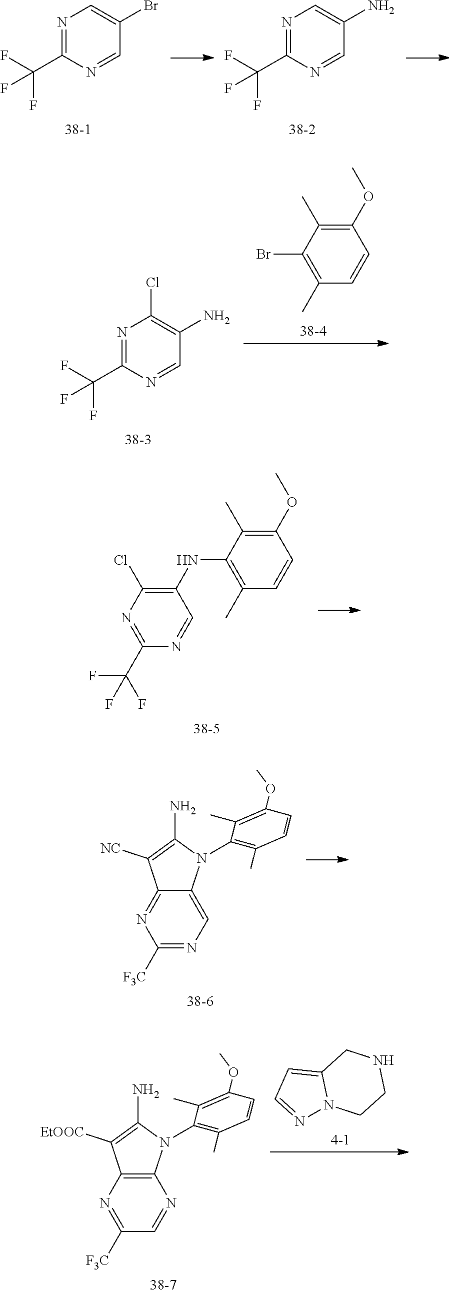

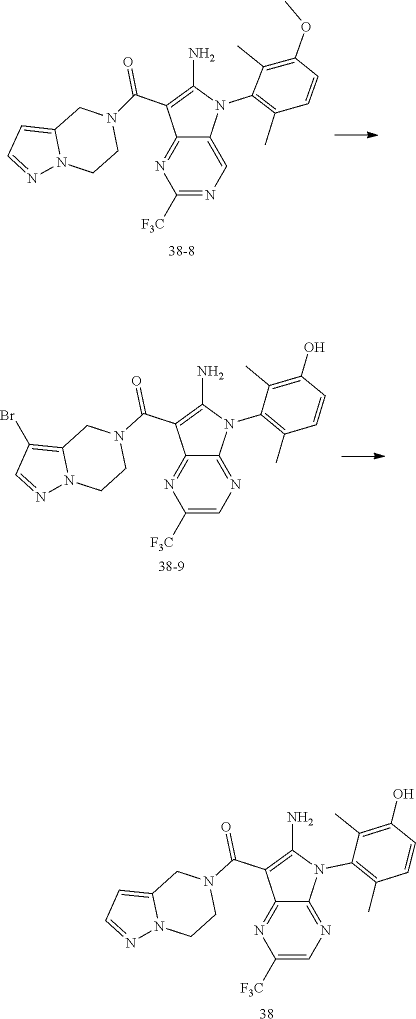

### Step 1: Preparation of Compound 38-2

To a solution of compound 38-1 (4 g, 17.62 mmol) in DMSO (40 mL) were added CuI (667 mg, 3.53 mmol), L-proline (800 mg, 7.05 mmol), and K₂CO₃ (3.65 g, 26.43 mmol), and the mixture was stirred at rt for 5 min. Then ammonium hydroxide (1.02 mL, 26.43 mmol) was added. The mixture was stirred at 85° C. overnight. The reaction was diluted with EA (100 mL) and water (10 mL). The organic layer was separated, washed with brine, and concentrated in vacuo. The residue was purified using silica gel column chromatography and dried to afford compound 38-2 (2 g, 69.64%) as white solid. LCMS: 164.0 [M+H]⁺.

### Step 2: Preparation of Compound 38-3

To a mixture of compound 38-2 (2 g, 12.27 mmol) in DMF (10 mL) was added NCS (2.456 g, 18.39 mmol). The reaction was stirred at rt overnight. The reaction was diluted with EA (100 mL) and water (100 mL). The organic layer was separated, washed with brine (100 mL), and concentrated in vacuo. The residue was purified using silica gel column chromatography to afford compound 38-3 (1.5 g, 62.06%) as yellow oil. LCMS: 198.0 [M+H]⁺.

### Step 3: Preparation of Compound 38-5

To a solution of compound 38-3 (1.5 g, 7.61 mmol) in dioxane (12 mL) were added compound 38-4 (3.27 g, 15.22 mmol), Ruphos-Pd-G3 (764.35 mg, 0.91 mmol) and Cs₂CO₃ (7.44 g, 22.83 mmol). The reaction mixture was stirred at 100° C. for 1 h by microwave. The reaction was diluted with EA (100 mL) and water (100 mL). The organic layer was separated, washed with brine (100 mL), and concentrated in vacuo. The residue was purified using silica gel column chromatography to afford the title compound 38-5 (200 mg, 7.92%) as white solid. LCMS: 332.1 [M+H]⁺.

### Step 4: Preparation of Compound 38-6

To a solution of propanedinitrile (79 mg, 1.20 mmol) in DME (2 mL) was added NaH (72 mg, 1.80 mmol) at 0° C. The mixture was stirred for 0.5 h. Then XantPhos-Pd-G3 (114 mg, 0.12 mmol) and compound 38-5 (200 mg, 0.60 mmol) were added. The mixture was stirred at 100° C. for 1 h by microwave. The mixture was extracted with DCM (20 mL×3). The combined organic phase was washed with brine (20 mL), dried over Na₂SO₄, filtered, concentrated, and purified by column chromatography to afford compound 38-6 (120 mg, 55% yield) as a white solid. LCMS: 362.3 [M+H]⁺.

### Step 5: Preparation of Compound 38-7

To a solution of compound 38-6 (120 mg, 0.33 mmol) in a H₂SO₄ solution (2 mL, 10% H₂SO₄ in EtOH) was stirred at 90° C. for 18 h. The mixture was added to ice water (100 mL), which was neutralized to pH=7 with NaOH (4 M in water), extracted with DCM (20 mL×3). The combined organic phase was washed with brine (20 mL), dried over anhydrous Na₂SO₄, filtered, concentrated, and purified by column chromatography to afford compound 38-7 (15 mg, 11.14% yield) as a white solid. LCMS: 409.4 [M+H]⁺.

### Step 6: Preparation of Compound 38-8

To a solution of compound 38-7 (15 mg, 0.036 mmol) in dioxane (1 mL) were added compound 4-1 (81 mg, 0.66 mmol), AlMe₃ (1.7 mL, 2 M). The reaction was stirred at 100° C. for 1 h. MeOH (4 mL) was added to quench the reaction. The mixture was filtered. The filtrate was dried to afford crude compound 38-8. LCMS: 486.4 [M+H]⁺.

### Step 7: Preparation of Compound 38-9

To a solution of compound 38-8 (15 mg) in DCM (2 mL) was added BBr₃ (0.1 mL). The reaction mixture was stirred at rt for 1 h. The reaction was quenched with MeOH (lmL). The mixture was concentrated to afford the crude compound 38-9. LCMS: 551.8 [M+H]⁺.

### Step 8: Preparation of Example 38

To a solution of the crude compound 38-9 (15 mg) in MeOH (5 mL) was added Pd/C (50 mg). The reaction mixture was stirred at rt for 18 h under H₂. The organic layer was separated, and concentrated to dryness. The residue was purified by Pre-HPLC to afford example 38. LCMS: 472.2 [M+H]⁺; ¹H NMR: (400 MHz, DMSO-d₆) δ 9.74 (s, 1H), 7.88 (s, 1H), 7.49 (s, 2H), 7.46 (s, 1H), 7.15 (d, J=8.4 Hz, 1H), 7.00 (d, J=8.5 Hz, 1H), 6.17 (s, 1H), 4.84 (s, 2H), 4.45 (s, 2H), 4.09 (s, 2H), 1.82 (s, 3H), 1.74 (s, 3H).

### Example 39: Preparation of (6-amino-7-(3-hydroxy-2,6-dimethylphenyl)-2-methyl-7H-pyrrolo[2,3-d]pyrimidin-5-yl)(5,6-dihydro-[1,2,4]triazolo[1,5-a]pyrazin-7(8H)-yl)methanone

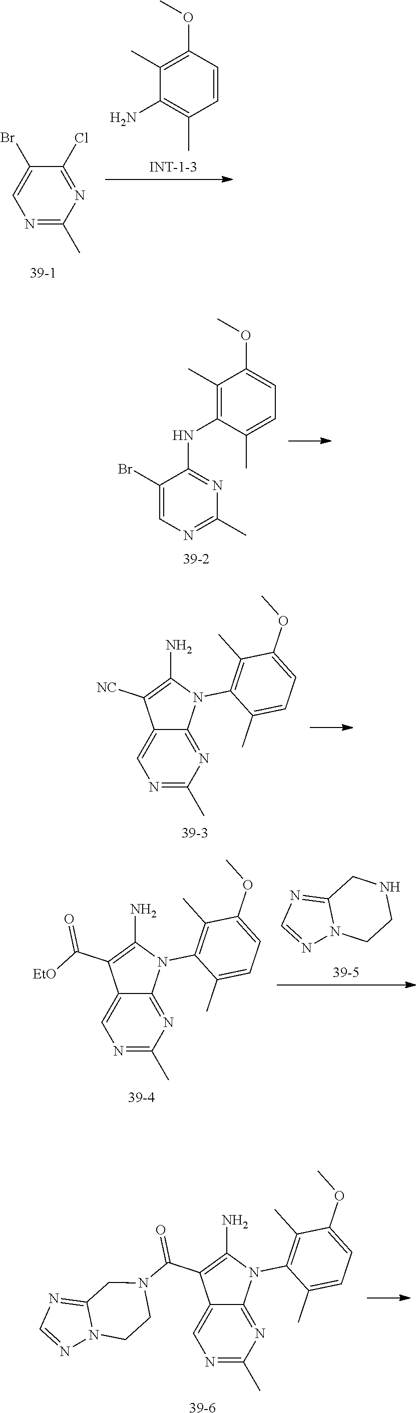

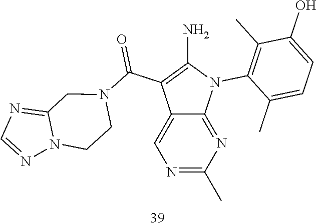 [SMILES: Cc1ncc2c(C(=O)N3CCn4ncnc4C3)c(N)n(-c3c(C)ccc(O)c3C)c2n1 ; "39"]

### Step 1: Preparation of Compound 39-2

To a solution of compound 39-1 (400 mg, 1.93 mmol) in t-BuOH (2 mL) were added compound INT-1-3 (292 mg, 1.93 mmol), TFA (220 mg, 1.93 mmol). The reaction was stirred at 100° C. overnight. The reaction was diluted with DCM (30 mL) and water (30 mL). The organic layer was separated and concentrated in vacuo. The residue was purified using silica gel column chromatography. The organic layer was collected, concentrated in vacuo, and dried to afford compound 39-2 (320 mg, 51.51%) as a white solid. LCMS: 322.2 [M+H]⁺.

### Step 2: Preparation of Compound 39-3

To a solution of compound 39-2 (600 mg, 1.86 mmol) in DME (1 mL) were added propanedinitrile (246 mg, 3.72 mmol), XantPhos-Pd-G3 (136 mg, 0.19 mmol), and NaH (98 mg, 4.10 mmol). The reaction was stirred at 110 T for 1 h. The reaction was diluted with DCM (30 mL) and water (30 mL). The organic layer was separated, and concentrated in vacuo. The residue was purified using silica gel column chromatography to afford compound 39-3 (330 mg, 57.66%) as a yellow solid. LCMS: 308.1 [M+H]⁺.

### Step 3: Preparation of Compound 39-4

A solution of compound 39-3 (200 mg, 0.651 mmol) in EtOH (3.5 mL) and H₂SO₄ (0.5 mL) was stirred at 90° C. for 16 h. The reaction was quenched by pouring into ice-water, which was extracted with DCM (50 mL). The organic layer was separated, washed with further saturated NaHCO₃ solution, and concentrated in vacuo. The residue was purified using silica gel column chromatography to afford compound 39-4. LCMS: 355.3 [M+H]⁺.

### Step 4: Preparation of Compound 39-6

To a solution of compound 39-4 (70 mg, 0.198 mmol) in dioxane (2 mL) were added compound 39-5 (36.78 mg, 0.296 mmol), and Me₃A1 (1.975 mL). The reaction was stirred at 120° C. for 48 h. The reaction was quenched with MeOH (10 mL), which was filtered. The filtrate was concentrated in vacuo. The residue was purified using silica gel column chromatography to afford compound 39-6. LCMS: 433.2 [M+H]⁺.

### Step 5: Preparation of Example 39

To a solution of compound 39-6 (20 mg, 0.046 mmol) in DCM (3 mL) was added BBr₃ (17.38 mg, 0.069 mmol). The reaction was stirred at room temperature for 1 h. The reaction was quenched with MeOH (10 mL), which was filtered. The filtrate was concentrated in vacuo. The crude product was purified by Pre-HPLC to afford example 39. LCMS: 419.3 [M+H]⁺; ¹H NMR: (400 MHz, DMSO-d₆) δ 9.58 (s, 1H), 8.49 (s, 1H), 8.14 (s, 1H), 7.97 (s, 1H), 7.08 (d, J=8.3 Hz, 1H), 6.94 (d, J=8.3 Hz, 1H), 6.69 (s, 2H), 4.88 (s, 2H), 4.27 (t, J=5.4 Hz, 2H), 4.05 (d, J=5.0 Hz, 2H), 2.46 (s, 3H), 1.77 (s, 3H), 1.69 (s, 3H).

### Example 40: Preparation of (6-amino-5-(3-hydroxy-2,6-dimethylphenyl)-3-methyl-2-(prop-1-yn-1-yl)-5H-pyrrolo[2,3-b]pyrazin-7-yl)(6,7-dihydropyrazolo[1,5-a]pyrazin-5(4H)-yl)methanone

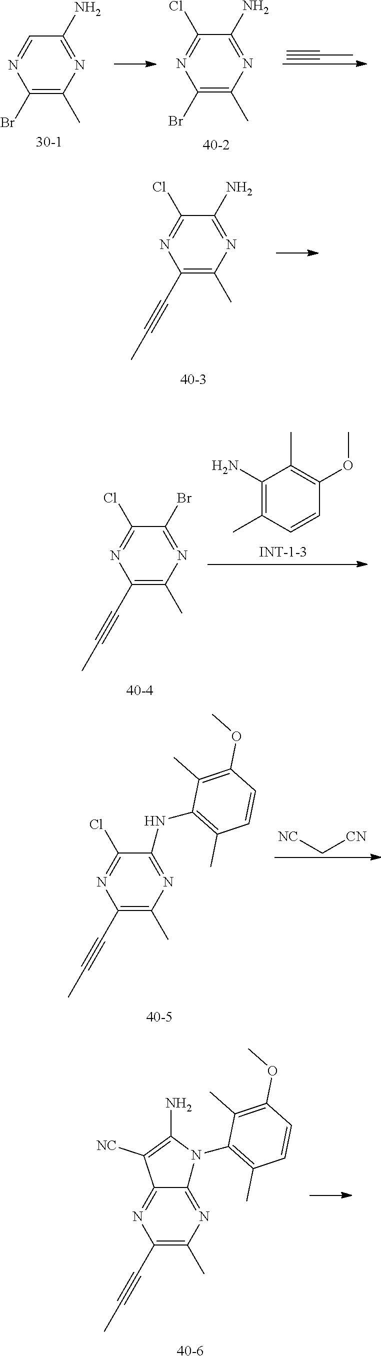

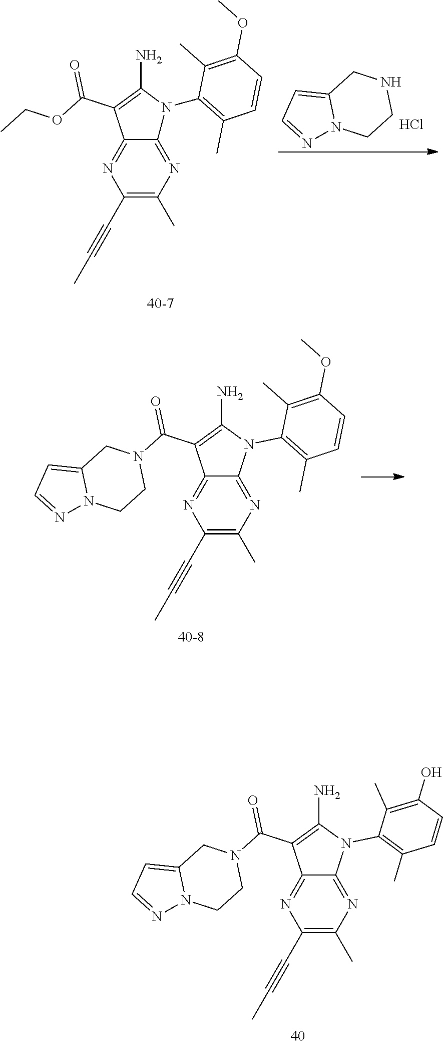

### Step 1: Preparation of Compound 40-2

To a solution of Compound 30-1 (5 g, 26.596 mmol) in AcOH (110 mL) was added N-chlorosuccinimide (3.6 g, 26.596 mmol). The mixture was stirred at 70° C. for 16 h. The mixture was concentrated, carefully added into ice-water (50 mL) while stirring, adjusted to pH-8 with Na₂CO₃ (aq), and extracted with EA (100 mL×3). The combined organic layers were washed with brine (300 mL), dried over Na₂SO₄, filtered, and concentrated to dryness. The residue was purified by column chromatography on silica gel to afford Compound 40-2 (5.2 g, 87.9%) as a yellow solid. LCMS: 224.0 [M+H]⁺.

### Step 2: Preparation of Compound 40-3

To a solution of Compound 40-2 (500 mg, 2.247 mmol) in TEA (10 mL) were added CuI (171 mg, 0.899 mmol), bis(triphenylphosphine)palladium(II) chloride (158 mg, 0.225 mmol) and propyne (ca. 5% in THF, ca. 1 mol/L) (180 mg, 4.494 mmol). The mixture was stirred at 70° C. for 16 h. The mixture was concentrated. The residue was purified by silica gel column chromatography to afford Compound 40-3 as a yellow solid. LCMS: 182.0 [M+H]⁺.

### Step 3: Preparation of Compound 40-4

A solution of Compound 40-3 (1000 mg, 5.507 mmol) and isopentyl nitrite (19.4 mg, 0.165 mmol) in CH₃CN (12 mL) was stirred at rt for 30 min. CuBr (1185 mg, 8.260 mmol) was added at 0° C. The mixture was stirred at 60° C. for 2 h under N₂ atmosphere and concentrated. The residue was purified by silica gel column chromatography to afford Compound 40-4 (920 mg, 45.4%) as a yellow solid. LCMS: 247.0 [M+H]⁺.

### Step 4: Preparation of Compound 40-5

To a solution of Compound 40-4 (300 mg, 1.222 mmol) and INT-1-3 (185 mg, 1.222 mmol) in toluene (3 mL) was added Pd₂(dba)₃ (111.9 mg, 0.122 mmol), Xantphos (141 mg, 0.244 mmol), Cs₂CO₃ (995 mg, 3.055 mmol) under N₂ atmosphere. The mixture was stirred at 100° C. for 1 h, and concentrated. The residue was purified by silica gel column chromatography and Pre-TLC to afford Compound 40-5 (70 mg, 18.1%) as a yellow solid. LCMS: 316.1 [M+H]⁺.

### Step 5: Preparation of Compound 40-6

To a solution of propanedinitrile (40 mg, 0.598 mmol) in DMF (1 mL) was added NaH (23 mg, 0.570 mmol). The mixture was stirred at 0° C. for 0.5 h. Compound 40-5 (90 mg, 0.285 mmol) and Pd(dppf)Cl₂·CH₂Cl₂ (23 mg, 0.028 mmol) was added. The resulting mixture was stirred at 90 T for 3 h. The mixture was diluted with EA (30 mL) and washed with brine (10 mL×3). The combined organic layers were dried over Na₂SO₄, filtered, and concentrated to dryness. The residue was purified by silica gel column chromatography to afford Compound 40-6 (90 mg, 91.4%) as a yellow solid. LCMS: 346.3 [M+H]⁺.

### Step 6: Preparation of Compound 40-7

A mixture of Compound 40-6 (70 mg, 0.203 mmol) in EtOH (1.8 mL) and H₂SO₄ (0.26 mL) was stirred at 90° C. for 16 h. The reaction mixture was carefully added into ice-water (20 mL) and adjusted to pH˜8 with NaOH (4M in water). The mixture was extracted with EA (30 mL×2). The combined organic layers were washed with brine (50 mL), dried over Na₂SO₄, filtered, and concentrated to dryness. The residue was purified by Pre-TLC to afford Compound 40-7. LCMS: 393.2 [M+H]⁺.

### Step 7: Preparation of Compound 40-8

To a solution of 4,5,6,7-tetrahydropyrazolo[1,5-a]pyrazine (9 mg, 0.076 mmol) in dioxane (0.5 mL) were added DIPEA (8 mg, 0.064 mmol), Compound 40-7 (10 mg, 0.025 mmol) and trimethylaluminium (0.06 mL, 0.127 mmol). The mixture was stirred at 100° C. for 2 h. The mixture was quenched by MeOH, and concentrated. The residue was purified by Pre-TLC to afford Compound 40-8. LCMS: 470.2 [M+H]⁺.

### Step 8: Preparation of Example 40

A mixture of Compound 40-8 (6 mg, 0.013 mmol) in DCM (3 mL) was added BBr₃ (0.01 mL, 0.128 mmol). The mixture was stirred at rt for 4 h, quenched with MeOH (10 mL), and concentrated to dryness. The residue was purified by prep-HPLC to afford Example 40. LCMS: 456.3 [M+H]⁺; HNMR: (400 MHz, MeOD) δ 7.50 (s, 1H), 7.11 (d, J=8.2 Hz, 1H), 6.94 (d, J=8.4 Hz, 1H), 6.18 (s, 1H), 4.59 (s, 2H), 4.52 (t, J=5.2 Hz, 2H), 4.19 (t, J=5.3 Hz, 2H), 2.51 (s, 3H), 2.14 (s, 3H), 1.88 (s, 3H), 1.82 (s, 3H).

### Example A: PKMYT1 HTRF Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25° C. Then 5 μL/well of tracer solution (Tracer 178) was added to initiate the reaction, and incubate for 60 minutes at 25° C. Next 5 μL GST-Tb was added into the assay plate, the plate was centrifuged at 1000 rpm for 1 minute, and incubate for 15 minutes at 25° C. The assay plate was read on Envision.

### Example B: WEE1 ADP-Glo Assay

Compound serial dilution is performed by Echo, and the final concentrations vary from 10 μM to 0.5 nM. This was filled by the addition of 5 μL/well of Enzyme solution to the assay plate containing the compound. The plate was centrifuged at 1000 rpm for 1 minute, and incubate 15 minutes at 25° C. Then 5 μL/well of substrate solution was added to initiate the reaction, and incubate for 60 minutes at 25° C. Next 10 μL kinase detection reagent was added into the assay plate, the plate was centrifuged at 1000 rpm for 1 minute, and incubate for 60 minutes at 25 T. The assay plate was read on Envision for US LUM as RLU.

The data for Example A and Example B is shown in Table 3.

TABLE 3

| | MYT1 HTRF | WEE1 ADP-Glo |
|---|---|---|
| Ex. | IC₅₀ (nM) | IC₅₀ (nM) |
| 1 | A | >10000 |
| 2 | B | >10000 |
| 3 | A | >10000 |
| 4 | A | >10000 |
| 5 | B | >10000 |
| 6 | B | >10000 |
| 7 | A | >10000 |
| 8 | B | >10000 |
| 9 | B | >10000 |
| 10 | B | >10000 |
| 11 | B | >10000 |
| 12 | B | >10000 |
| 13 | A | >10000 |
| 14 | A | >10000 |
| 15 | A | >10000 |
| 16 | A | >10000 |
| 17 | A | >10000 |
| 18 | A | >10000 |
| 19 | D | >10000 |
| 20 | A | >10000 |
| 21 | B | >10000 |
| 22 | A | >10000 |
| 23 | A | >10000 |
| 24 | B | 4547 |
| 25 | B | >10000 |
| 26 | D | >10000 |
| 27 | C | >10000 |
| 28 | A | 5430 |
| 29 | B | >10000 |
| 30 | A | >10000 |
| 31 | A | 4319 |
| 32 | A | >10000 |
| 33 | B | >10000 |
| 34 | C | >10000 |
| 35 | B | >10000 |
| 36 | C | >10000 |
| 37 | B | 9135 |
| 38 | C | >10000 |
| 39 | C | >10000 |
| 40 | B | Not tested |

MYT1 HTRF IC₅₀ (nM):
0<A≤10;
10<B≤50;
50<C≤500;
500<D≤5000;
5000<E

What is claimed is:

1. A compound of Formula (I), or a pharmaceutically acceptable salt thereof: 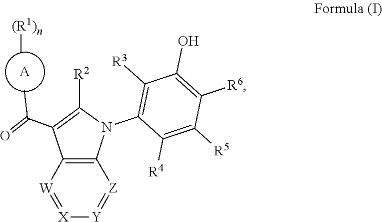
wherein:
Ring A is heterocycloalkyl or heteroaryl;
each R¹ is independently halogen, —CN, —NO₂, —OH, —OR^{a}, —OC(═O)R^{a}, —OC(═O)OR^{b}, —OC(═O)NR^{c}R^{d}, —SH, —SR, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more Ria;
or two R¹ on the same atom are taken together to form an oxo;
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{1a} on the same atom are taken together to form an oxo;
n is 0-6;
R² is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
W is N or CR^{W};
R^{W} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
X is N or CR^{X};
R^{X} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Xa};
each R^{Xa} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₅haloalkyl, C₁-C₆hydroxyalkyl, C₁-C₆aminoalkyl, C₁-C₆heteroalkyl, C₂-C₆alkenyl, C₂-C₅alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Xa} on the same atom are taken together to form an oxo;
Y is N or CR^{Y};
R^{Y} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Ya};
each R^{Ya} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{Ya} on the same atom are taken together to form an oxo;
Z is N or CR^{Z};
R^{Z} is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R;
R³ is halogen, C₁-C₆alkyl, C₁-C₆haloalkyl, C₁-C₆hydroxyalkyl, C₁-C₅aminoalkyl, or C₁-C₆heteroalkyl, cycloalkyl, or heterocycloalkyl;
R⁴ is halogen, C₁-C₆alkyl, C₁-C₆haloalkyl, C₁-C₆hydroxyalkyl, C₁-C₆aminoalkyl, or C₁-C₆heteroalkyl, cycloalkyl, or heterocycloalkyl;
R¹ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, or C₃-C₆cycloalkyl;
or two R on the same atom form an oxo.

2. The compound of claim 1, or a pharmaceutically acceptable salt thereof, wherein X is N.

3. The compound of claim 1, or a pharmaceutically acceptable salt thereof, wherein X is CR^{X}.

4. The compound of any one of claims 1-3, or a pharmaceutically acceptable salt thereof, wherein Y is N.

5. The compound of any one of claims 1-3, or a pharmaceutically acceptable salt thereof, wherein Y is CR^{Y}.

6. The compound of any one of claims 1-5, or a pharmaceutically acceptable salt thereof, wherein W is N.

7. The compound of any one of claims 1-5, or a pharmaceutically acceptable salt thereof, wherein W is CR^{W}.

8. The compound of any one of claims 1-7, or a pharmaceutically acceptable salt thereof, wherein Z is N.

9. The compound of any one of claims 1-7, or a pharmaceutically acceptable salt thereof, wherein Z is CR^{Z}.

10. The compound of claim 1, or a pharmaceutically acceptable salt thereof, wherein the compound is of Formula (Ia):

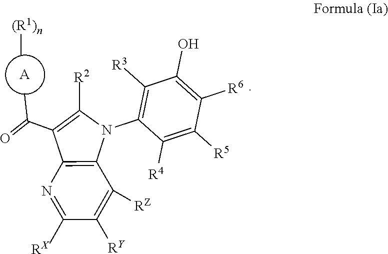

11. The compound of claim 1, or a pharmaceutically acceptable salt thereof, wherein the compound is of Formula (Ib):

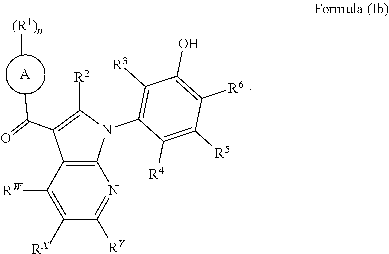

12. The compound of claim 1, or a pharmaceutically acceptable salt thereof, wherein the compound is of Formula (Ic):

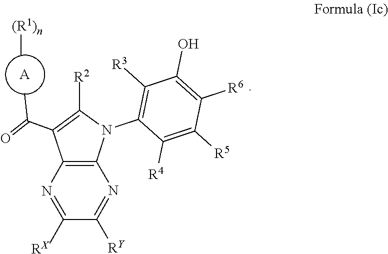

13. The compound of claim 1, or a pharmaceutically acceptable salt thereof, wherein the compound is of Formula (Id):

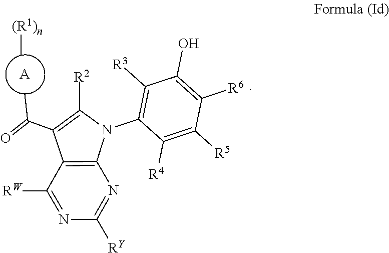

14. The compound of claim 1, or a pharmaceutically acceptable salt thereof, wherein the compound is of Formula (Ie):

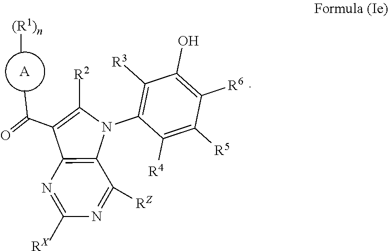

15. The compound of any one of claims 1-14, or a pharmaceutically acceptable salt thereof, wherein R³ is halogen, C₁-C₆alkyl, or C₁-C₆haloalkyl.

16. The compound of any one of claims 1-15, or a pharmaceutically acceptable salt thereof, wherein R³ is C₁-C₆alkyl.

17. The compound of any one of claims 1-16, or a pharmaceutically acceptable salt thereof, wherein R⁴ is halogen, C₁-C₆alkyl, or C₁-C₆haloalkyl.

18. The compound of any one of claims 1-17, or a pharmaceutically acceptable salt thereof, wherein R⁴ is C₁-C₆alkyl.

19. The compound of any one of claims 1-18, or a pharmaceutically acceptable salt thereof, wherein R⁵ is hydrogen.

20. The compound of any one of claims 1-19, or a pharmaceutically acceptable salt thereof, wherein R⁶ is hydrogen.

21. The compound of any one of claims 1-20, or a pharmaceutically acceptable salt thereof, wherein R² is —NR^{c}R^{d}.

22. The compound of any one of claims 1-21, or a pharmaceutically acceptable salt thereof, wherein R^{W} is hydrogen, halogen, —CN, —OH, —OR^{a}, —NR^{c}R^{d}, C₁-C₆alkyl, or C₁-C₆haloalkyl.

23. The compound of any one of claims 1-22, or a pharmaceutically acceptable salt thereof, wherein R^{W} is hydrogen.

24. The compound of any one of claims 1-23, or a pharmaceutically acceptable salt thereof, wherein R^{X} is hydrogen, halogen, —CN, —OH, —OR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally substituted with one or more R^{Xa}.

25. The compound of any one of claims 1-24, or a pharmaceutically acceptable salt thereof, wherein R^{X} is hydrogen, halogen, C₁-C₆alkyl, C₁-C₆haloalkyl, or heterocycloalkyl; wherein the alkyl and heterocycloalkyl is optionally substituted with one or more R^{Xa}.

26. The compound of any one of claims 1-25, or a pharmaceutically acceptable salt thereof, wherein R^{X} is hydrogen, halogen, C₁-C₆alkyl, or C₁-C₆haloalkyl.

27. The compound of any one of claims 1-26, or a pharmaceutically acceptable salt thereof, wherein R^{X} is halogen.

28. The compound of any one of claims 1-27, or a pharmaceutically acceptable salt thereof, wherein R^{Y} is hydrogen, halogen, —CN, —OH, —OR^{a}, —NR^{c}R^{d}, C₁-C₆alkyl, C₁-C₆haloalkyl, cycloalkyl, or heterocycloalkyl; wherein the alkyl, cycloalkyl, and heterocycloalkyl is optionally substituted with one or more R^{Ya}.

29. The compound of any one of claims 1-28, or a pharmaceutically acceptable salt thereof, wherein R^{Y} is hydrogen, halogen, C₁-C₆alkyl, or heterocycloalkyl; wherein the alkyl and heterocycloalkyl is optionally substituted with one or more R^{Ya}.

30. The compound of any one of claims 1-29, or a pharmaceutically acceptable salt thereof, wherein R^{Y} is hydrogen or C₁-C₆alkyl.

31. The compound of any one of claims 1-30, or a pharmaceutically acceptable salt thereof, wherein R^{Z} is hydrogen, halogen, —CN, —OH, —OR^{a}, —NR^{c}R^{d}, C₁-C₆alkyl, or C₁-C₆haloalkyl.

32. The compound of any one of claims 1-31, or a pharmaceutically acceptable salt thereof, wherein R^{Z} is hydrogen.

33. The compound of any one of claims 1-32, or a pharmaceutically acceptable salt thereof, wherein Ring A is heterocycloalkyl.

34. The compound of any one of claims 1-33, or a pharmaceutically acceptable salt thereof, wherein Ring A is bicyclic heterocycloalkyl.

35. The compound of any one of claims 1-34, or a pharmaceutically acceptable salt thereof, wherein Ring A is 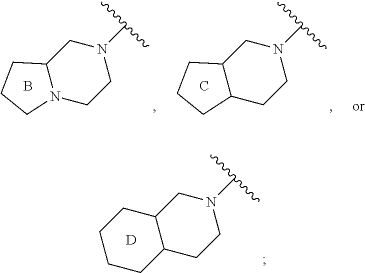
wherein Ring B is a 5-membered heteroaryl, Ring C is a 5-membered heteroaryl, and Ring D is a 6-membered heteroaryl.

36. The compound of any one of claims 1-35, or a pharmaceutically acceptable salt thereof, wherein Ring A is

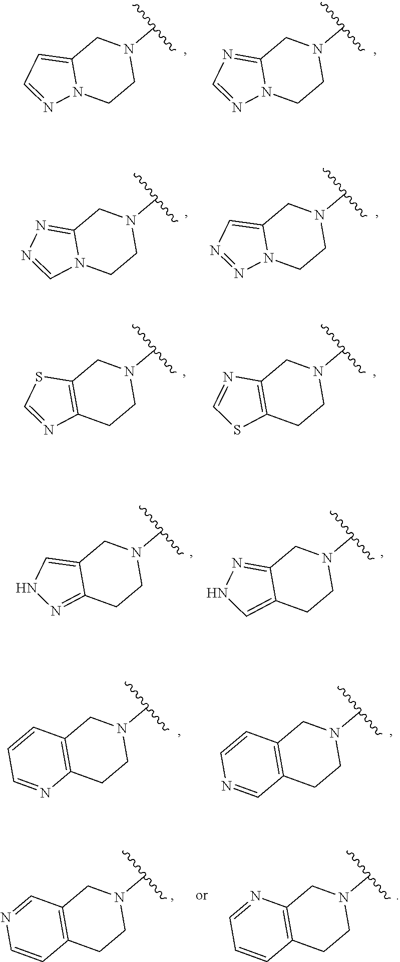

37. The compound of any one of claims 1-36, or a pharmaceutically acceptable salt thereof, wherein Ring A is

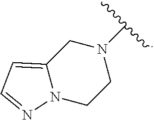 [SMILES: *N1CCn2nccc2C1]

38. The compound of any one of claims 1-37, or a pharmaceutically acceptable salt thereof, wherein each R¹ is independently halogen, —CN, —OH, —OR^{a}, —NR^{c}R^{d}, C₁-C₆alkyl, or C₁-C₆haloalkyl.

39. The compound of any one of claims 1-38, or a pharmaceutically acceptable salt thereof, wherein each R¹ is independently halogen, C₁-C₆alkyl, or C₁-C₆haloalkyl.

40. The compound of any one of claims 1-39, or a pharmaceutically acceptable salt thereof, wherein n is 0-2.

41. The compound of any one of claims 1-40, or a pharmaceutically acceptable salt thereof, wherein n is 1 or 2.

42. The compound of any one of claims 1-41, or a pharmaceutically acceptable salt thereof, wherein n is 1.

43. The compound of claim 1, or a pharmaceutically acceptable salt thereof, wherein the compound is selected from a compound found in table 1 or table 2.

44. A pharmaceutical composition comprising the compound of any one of claims 1-43, or a pharmaceutically acceptable salt thereof, and a pharmaceutically acceptable excipient.

45. A method of treating cancer in a subject in need thereof, the method comprising administering to the subject the compound of any one of claims 1-43, or a pharmaceutically acceptable salt thereof.

46. A method of modulating PKMYT1 in a subject, the method comprising administering to the subject the compound of any one of claims 1-43, or a pharmaceutically acceptable salt thereof.

47. A method of inhibiting PKMYT1 in a subject, the method comprising administering to the subject the compound of any one of claims 1-43, or a pharmaceutically acceptable salt thereof.

48. The method of claim 46 or 47, wherein the compound or a pharmaceutically acceptable salt thereof does not inhibit WEE1.

49. The method of any one of claims 46-48, wherein the compound or a pharmaceutically acceptable salt thereof has a WEE1 IC₅₀ value of at least about 10,000 nM as determined in a WEE1 ADP-Glo assay.

50. The method of any one of claims 46-49, wherein the subject has cancer.

51. The method of claim 45 or 50, wherein the cancer depends on the activity of PKMYT1.

52. The method of any one of claims 45, 50, or 51, wherein the cancer overexpresses CCNE1.

53. The method of any one of claims 45 or 50-52, wherein the cancer has an inactivating mutation in the FBXW7 gene.

54. The method of any one of claims 45 or 50-53, wherein the cancer is a solid tumor.

55. The method of any one of claims 45 or 50-54, wherein the cancer is breast cancer, colorectal cancer, endometrial cancer, esophageal cancer, glioblastoma, hepatocellular carcinoma, lung cancer, neuroblastoma, ovarian cancer, prostate cancer, stomach cancer, or uterine cancer.
